(19) United States
(12) Patent Application Publication
(10) Pub. No.: US 2025/0242017 A1
(43) Pub. Date: Jul. 31, 2025

(54) HISTAMINE AND HRH1 SIGNALING ACTIVITY AS BIOMARKERS AND ASSOCIATED METHODS

(71) Applicant: Board of Regents, The University of Texas System, Austin, TX (US)

(72) Inventors: Yi XIAO, Houston, TX (US); Xiangliang YUAN, Houston, TX (US); Dihua YU, Houston, TX (US); Hongzhong LI, Chongqing (CN); Qin LI, Beijing (CN)

(21) Appl. No.: 18/155,510

(22) Filed: Jan. 17, 2023

Related U.S. Application Data
(60) Provisional application No. 63/299,736, filed on Jan. 14, 2022.

(51) Int. Cl. A61K 39/395; A61K 45/06; A61P 35/00; G01N 33/574

(52) U.S. Cl. CPC A61K 39/3955; A61K 45/06; A61P 35/00; G01N 33/57488

(57) ABSTRACT

A method of determining the likelihood that a subject with cancer will respond to an immunotherapy comprising a checkpoint inhibitor. The method comprises detecting the level of histamine in a biological sample obtained from the subject. Subjects determined to have a low likelihood of responding to the immunotherapy can be administered an alternative therapy or administered an antihistamine in conjunction with the immunotherapy. Methods of treating cancer patients are also provided, as are kits and systems for histamine detection, recordation, and responder status determination.

### CROSS-REFERENCES TO RELATED APPLICATIONS

This application claims priority to and the benefit of U.S. Provisional Patent Application Ser. No. 63/299,736, filed Jan. 14, 2022, the contents of which are incorporated herein by this reference as if fully set forth herein.

### STATEMENT AS TO RIGHTS TO INVENTIONS MADE UNDER FEDERALLY SPONSORED RESEARCH AND DEVELOPMENT

This invention was made with Government support under grant numbers R01 CA231149 and R01 CA208213 awarded by the National Institutes of Health. The Government has certain rights in the invention.

### BACKGROUND

T cell-mediated anti-tumor immunity plays a central role in host's defense against cancer. However, cancer cells can coevolve with the tumor immune microenvironment and develop different strategies to evade T cell immune destruction. Tumor infiltrating T cells often manifest impaired effector function (i.e., dysfunction) and fail to eliminate cancer cells owing to various T-cell-inhibitory signals, e.g., cytotoxic lymphocyte antigen-4 (CTLA-4) and programmed cell death protein 1 (PD-1)/programmed death ligand 1 (PD-L1). Anti-CTLA-4 and anti-PD1/PDL1 antibodies, as immune checkpoint therapies (ICT; also referred to as Immune Checkpoint Blockade, or ICB therapies), have yielded significant clinical benefits and durable responses in a subset of cancer patients. Yet, most cancer patients cannot benefit from these treatments, and it is highly challenging to reach immunotherapy's full potential. Importantly, there is an unmet challenge to develop reliable biomarkers for assessing patients who will benefit from immunotherapy treatment versus those who need rationally designed combinatory immunotherapies.

Histamine, a metabolite of histidine, is best known for its release from mast cells as a response to allergic reactions or tissue damage. Histamine exerts its effects primarily by binding to G-protein-coupled receptors, designated histamine receptors H1 through H4 (HRH1/2/3/4). Among them, HRH1 is the major one involved in allergic response. During allergic reactions, mast cell-released histamines activate HRH1, which triggers contraction of smooth muscles and increases capillary permeability, resulting in classic allergy symptoms. HRH1 antagonists, mostly over-the-counter (OTC) drugs, are widely used to relieve allergy symptoms and to prevent nausea and vomiting in cancer treatment.

There is therefore an unmet need in the art for reliable biomarkers for assessing patients who will benefit from immunotherapy treatment versus those who need rationally-designed combinatory immunotherapies, as well as for therapeutics that can enhance the efficacy of such treatments. The present disclosure addresses this need and provides other advantages as well.

### BRIEF SUMMARY

In one aspect, the present disclosure provides a method of determining the likelihood that a subject with cancer will respond to an immunotherapy comprising the administration of a checkpoint inhibitor, the method comprising detecting a level of histamine and/or HRH1 signaling activation in a biological sample obtained from the subject, wherein a detection of an elevated level of histamine and/or HRH1 signaling activation indicates that the subject is unlikely to respond to the immunotherapy.

In some embodiments, the method further comprises calculating a response score based on the detected level of the histamine, wherein the response score corresponds to the likelihood that the subject will respond to the therapy. In some embodiments, the subject is determined to have a high likelihood of responding to the therapy. In some embodiments, the method further comprises administering the immunotherapy to the subject. In some embodiments, the subject is determined to have a low likelihood of responding to the therapy. In some embodiments, the method further comprises administering an alternative anticancer therapy to the subject that does not comprise the immunotherapy, and/or administering an antihistamine to the subject prior to or in conjunction with the immunotherapy. In some embodiments, the checkpoint inhibitor is an inhibitor of PD-1, PD-L1, or CTLA-4.

In some embodiments, the histamine level is measured by detecting histamine in a plasma sample obtained from the subject, and/or by detecting histamine, HDC mRNA, HDC protein, and/or mast cells in a tumor sample obtained from the subject. In some embodiments, the level of HRH1 signaling activation is detected by detecting the level of HRH1 mRNA, HRH1 protein, HRH1+ macrophages, VISTA+ macrophages, PI3K-gamma+ macrophages, and/or TIM-3+ macrophages in a tumor sample obtained from the subject. In some embodiments, the level of histamine, HDC protein, HRH1 protein, HRH1+ macrophages, VISTA+ macrophages, PI3K-gamma+ macrophages, TIM-3+ macrophages, and/or mast cells is detected by immunohistochemistry (IHC) staining, flow cytometry staining, and/or by ELISA. In some embodiments, the level of histamine and/or HRH1 signaling activation is detected indirectly by virtue of the presence of allergies in the subject and/or by detecting plasma IgE levels.

In some embodiments, the antihistamine is an H1-antihistamine. In some embodiments, the H1-antihistamine is selected from the group consisting of Cetirizine, Loratadine, Ketotifen, Rupatadine, Bilastine, Terfenadine, Astemizole, Mizolastine, Acrivastine, Ebastine, Bepotastine, Azelastine, Levocabastine, Olopatadine, Levocetirizine, Desloratadine, Fexofenadine, Quifenadine, and derivatives thereof. In some embodiments, the derivative is an intravenous injection form of the H1-antihistamine. In some embodiments, the intravenous injection form is Quzyttir. In some embodiments, the antihistamine is an inhibitor of HDC-mediated histamine production or of a downstream effector of HRH1 signaling activation. In some embodiments, the downstream effector is VISTA, PI3K-gamma, or TIM-3. In some embodiments, the cancer is associated with high levels of histamine production, HRH1 signaling activation, and/or plasma histamine. In some embodiments, the cancer is colorectal cancer, breast cancer, lung cancer, or malignant melanoma.

In another aspect, the present disclosure provides a method of treating cancer in a subject, the method comprising determining that the subject has at least one of an elevated level of histamine or HRH1 signaling activation, and administering to the subject an antihistamine in conjunction with an immunotherapy comprising the administration of a checkpoint inhibitor.

In some embodiments, the checkpoint inhibitor is an inhibitor of PD-1, PD-L1, or CTLA-4. In some embodiments, the subject is determined to have an elevated level of histamine by detecting histamine in a plasma sample obtained from the subject, and/or by detecting histamine, HDC mRNA, HDC protein, and/or mast cells in a tumor sample obtained from the subject. In some embodiments, the subject is determined to have an elevated level of HRH1 signaling activation by detecting the level of HRH1 mRNA, HRH1 protein, HRH1+ macrophages, VISTA+ macrophages, PI3K-gamma+ macrophages, and/or TIM-3+ macrophages in a tumor sample obtained from the subject. In some embodiments, the level of histamine, HDC protein, HRH1 protein, IgE, HRH1+ macrophages, VISTA+ macrophages, PI3K-gamma+ macrophages, TIM-3+ macrophages, and/or mast cells is detected by immunohistochemistry (IHC) staining, flow cytometry staining, and/or ELISA. In some embodiments, the subject is determined to have an elevated level of histamine or HRH1 signaling activation by virtue of the presence of allergies and/or by detection of plasma IgE levels in the subject.

In some embodiments, the antihistamine is an H1-antihistamine. In some embodiments, the H1-antihistamine is selected from the group consisting of Cetirizine, Loratadine, Ketotifen, Rupatadine, Bilastine, Terfenadine, Astemizole, Mizolastine, Acrivastine, Ebastine, Bepotastine, Azelastine, Levocabastine, Olopatadine, Levocetirizine, Desloratadine, Fexofenadine, Quifenadine, and derivatives thereof. In some embodiments, the derivative is an intravenous injection form of the H1-antihistamine. In some embodiments, the intravenous injection form is Quzyttir. In some embodiments, the antihistamine is an inhibitor of HDC-mediated histamine production or of a downstream effector of HRH1 activation. In some embodiments, the downstream effector is VISTA, PI3K-gamma, or TIM-3. In some embodiments, the cancer is associated with high levels of histamine production, HRH1 signaling activation, and/or plasma histamine. In some embodiments, the cancer is colorectal cancer, breast cancer, lung cancer, or malignant melanoma.

In another aspect, the present disclosure provides a method of generating a report containing information on the likelihood that a subject with cancer will respond to an immunotherapy comprising a checkpoint inhibitor, the method comprising detecting a level of histamine and/or HRH1 signaling activation in a biological sample obtained from the subject, and generating the report, wherein the report is useful for determining the likelihood that the subject will respond to the therapy.

In some embodiments, the biological sample is a plasma sample. In some embodiments, the histamine is detected in the plasma sample using ELISA. In some embodiments, the biological sample is a tumor sample, and wherein the level of histamine and/or HRH1 signaling activation is determined by detecting histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages in the sample.

In another aspect, the present disclosure provides a system for determining the likelihood that a subject with cancer will respond to an immunotherapy comprising a checkpoint inhibitor, comprising a station for analyzing a biological sample obtained from the subject to measure a level of histamine and/or HRH1 signaling activation in the sample.

In some embodiments, the biological sample is a plasma sample. In some embodiments, the histamine is detected in the plasma sample using ELISA. In some embodiments, the biological sample is a tumor sample, and wherein the level of histamine and/or HRH1 signaling activation is determined by detecting histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages in the sample. In some embodiments, the system further comprises a station for generating a report containing information on results of the analyzing.

In another aspect, the present disclosure provides a method of treating a subject with cancer, the method comprising administering to the subject a therapeutically effective amount of a checkpoint inhibitor and of an antihistamine, wherein the subject has been identified as unlikely to respond to an immunotherapy comprising the checkpoint inhibitor based on a detection of levels of histamine and/or HRH1 signaling activation in a biological sample obtained from the subject, wherein the identification of the subject as unlikely to respond to the immunotherapy is based on a difference in the level of histamine and/or HRH1 signaling activation in the biological sample obtained from the subject as compared to the level in a biological sample obtained from an individual known to be responsive to the immunotherapy.

In some embodiments, the identification of the subject as unlikely to respond to the immunotherapy comprises the calculation of a response score based on the detected levels of histamine and/or HRH1 signaling activation in the biological sample, wherein the response score corresponds to the likelihood that the subject will respond to the immunotherapy. In some embodiments, the checkpoint inhibitor is an inhibitor of PD-1, PD-L1, or CTLA-4. In some embodiments, the biological sample is a plasma sample. In some embodiments, the histamine is detected in the plasma sample using ELISA. In some embodiments, the biological sample is a tumor sample, and wherein the level of histamine and/or HRH1 signaling activation is determined by detecting histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages in the sample.

### BRIEF DESCRIPTION OF THE DRAWINGS

FIGS. 1A-1H. Uptake of antihistamines is correlated with better survival in ICB-treated patients and HRH1 expression is associated with T cell dysfunction according to aspects of this disclosure. FIG. 1A: Scatterplot of the real numbers of deceased melanoma patients who took various commonly used medicines (40 different drugs as listed in Table S1) during ICB treatment versus their estimated deaths (at 39% death rate based on 336 deceased patients of a total 865 ICB-treated patients at MDACC). Each dot represents a group of patients who took one type of medicine along with ICB. FIG. 1B: Percentages of deceased cancer patients taking H1-antihistamines during anti-PD-1/PD-L1 treatment versus those who did not (Fisher's exact test). FIG. 1C: Percentages of deceased melanoma patients who took H1-antihistamines during anti-PD-1/PD-L1 treatment or chemotherapy compared with sex- or stage matched melanoma patients who did not. FIG. 1D: Kaplan-Meier overall survival analysis of cancer patients taking H1-antihistamines during anti-PD-1/PD-L1 treatment versus those who did not. FIG. 1E: Percentages of deceased cancer patients taking H1-antihistamines during chemotherapy treatment versus those did not (Fisher's exact test). FIG. 1F: T cell dysfunction scores of HRH1-4 in indicated cancer types assessed by TIDE. T cell dysfunction score is defined as the Z score of d/standard error (s.e.), following a previous publication (Jiang et al., 2018). FIG. 1G: Kaplan-Meier overall survival analysis for CTL+ TNBC patients based on HRH1 level detected in tumors. The numbers at risk are the stratified HRH1-level high and low patient numbers of those who remained alive and uncensored after a certain time period. FIG. 1H: HRH1 mRNA expression in pre-treatment tumors of responder (n=15) versus non-responder (n=13) melanoma patients (t test) and comparison of overall survival of melanoma patients who had high HRH1 versus low HRH1 expression in the tumors before anti-PD-1 treatment (GSE78220). Mean±SEM; log-rank test for survival comparison. See also FIGS. 8A-8F and Tables S1 and S2.

FIGS. 2A-2I. Activated histamine-HRH axis in tumor microenvironment. FIG. 2A: Relative HRH1 mRNA levels in immune cell subsets assessed by CIBERSORT according to aspects of this disclosure. FIG. 2B: Flow cytometry analysis of HRH1 expression on human peripheral blood monocyte (PBMC)-derived macrophages (n=3, one-way ANOVA). FIG. 2C: Representative images and quantification of HRH1+ macrophages in human breast tissues (n=9) and TNBC tumors (n=32, t test). Scale bar, 25 mm. FIG. 2D: Percentage of CD68− or CD68+ cells (macrophages) in total HRH1+ cells in human TNBC tissues (n=32, t test). FIG. 2E: Flow cytometry analysis of HRH1 expression on mouse bone marrow-derived (BMDM) naive macrophages (M0) and polarized macrophages (M1 and M2-like) (n=3, one-way ANOVA). FIG. 2F: Mean fluorescence intensity (MFI) of HRH1 in indicated cell subsets of mousemammary tumors (4T1 and EO771). Macrophages, CD45+ CD11b+ Grl−F4/80+; neutrophils, CD45+ CD11b+ Grl+; lymphocytes, CD45+ CD11b−. FIG. 2G: Flow cytometry analysis of HRH1 expression on resident macrophages (nM4) from mammary fat pad (MFP) of BALB/c mice and TAMs (4T1 and EMT6 tumors) (n=5, one-way ANOVA). FIG. 2H: Histamine levels in blood plasma from healthy subjects (n=20), patients with TNBC (n=50), and patients with colon cancer (n=28) detected by ELISA (one way ANOVA). FIG. 2I: Pearson correlation analysis of the relationship between serum histamine level and GZMB+ cell density (%) in cancer tissues from TNBC patients (n=50). Mean±SEM; **p<0.01; ****p<0.0001. See also FIGS. 9A-9J.

FIGS. 3A-3H. Inhibiting HRH1 on macrophages enhances T cell antitumor immunity according to aspects of this disclosure. FIG. 3A: Flow cytometry analysis of M1-like (MHC II+) versus M2-like (CD206+) populations in bone marrow-derived macrophages (BMDMs) that were generated from wild-type (WT) or HRH1−/− mice and treated with vehicle or fexofenadine (FEXO) (10 mM) in the presence of EO771 tumor-cell-conditioned medium (TCM) for 48 h. FIG. 3B: Analysis of IFN-g+ CD8+ T cells in splenocytes co-cultured with vehicle- or FEXO (10 mM)-treated WT or HRH1−/− BMDMs (TCM-educated). FIGS. 3C-3D: MHC II:CD206 mean fluorescence intensity (MFI) ratios of tumor-associated macrophages (TAMs) (FIG. 3C) and percentage of IFN-g+ CD8+ T cells (FIG. 3D) in EO771 tumors (left) or B16-GM tumors (right) growing in WT versus HRH1−/− mice, and vehicle-treated versus FEXO-treated WT mice (n=5-6, t test). FIG. 3E: EO771 (left) and B16-GM (right) tumor growth in WT versus HRH1−/− mice, and vehicle-treated versus FEXO-treated WT mice (n=5-8 mice/group, twoway ANOVA). FIG. 3F: B16-GM tumor growth with indicated treatment. CD8+ T cells were depleted by anti-CD8 antibodies (n=6-7 mice/group, two-way ANOVA). FIG. 3G: Growth of B16-GM tumor cells co-implanted with WT or HRH1−/− BMDMs in HRH1−/− or WT recipient mice, respectively (n=6-9 mice/group, twoway ANOVA). FIG. 3H: Percentages of IFN-g+ and PRF1+ CD8+ T cells in primary tumors from WT and HRH1−/− mice transplanted with B16-GM tumor cells alone or both tumor cells and BMDMs (HRH1−/− or WT, respectively) (n=6, one-way ANOVA). Mean±SEM; *p<0.05; **p<0.01; ***p<0.001. See also FIGS. 10A-10M, 11A-11C, and 12A-12D.

FIGS. 4A-4E. Histamine-HRH1 activation promotes VISTA membrane localization according to aspects of this disclosure. FIG. 4A: Percentages of IFN-g+ CD8+ T cells co-cultured with EO771 TCM-treated WT or HRH1−/− BMDMs in direct contact or separately in transwells (n=6, t test). FIG. 4B: Percentages of IFN-g+ CD8+ T cells co-cultured with EO771 TCM-treated WT or HRH1−/− BMDMs pre-treated with IgG, anti-TIM-3 (10 mg/mL), and/or anti-VISTA (10 mg/mL) antibodies (n=3-4, one-way ANOVA). FIG. 4C: Flow cytometry analysis of VISTA+ TAMs (CD45+ CD11b+F4/80+) from EO771 and B16-GM tumors growing in WT versus HRH1−/− mice, and vehicle-treated versus FEXO-treated WT mice (n=5-6, t test). FIG. 4D: Western blot analysis of total VISTA and membrane VISTA expression on naive or TCM-treated WT or HRH1−/− BMDMs. b-actin and CD11b were used as loading controls. FIG. 4E: Percentages of VISTA+ BMDMs after treatment with 10 mM BAPTA-AM (an intracellular calcium chelator) or 1 mg/mL ionomycin-Ca2+ (n=3, t test). All in vitro experiments were performed at least twice. Mean±SEM; *p<0.05; **p<0.01; ***p<0.001; NS, not significant. See also FIGS. 13A-13L.

FIGS. 5A-5F: HRH1 knockout reshapes the transcriptomic landscape of macrophages according to aspects of this disclosure. FIG. 5A: Volcano plots of log 2 fold change (FC) and log 10 adjusted p value of differentially expressed genes between TCM-treated WT and HRH1−/− macrophages. Dots on the right third of the graph reflect genes upregulated in WT macrophages; dots on the left third of the graph reflect genes upregulated in HRH1−/− macrophages. FIG. 5B: The pathway enrichment map of differentially expressed genes between WT and HRH1−/− macrophages. FIG. 5C: Violin plot showing the expression-based score of M1-like (top) and M2-like macrophage (bottom), with their mean scores and p values labeled. The horizontal lines represent 25th percentile, median, and 75th percentile of the scores. Significance levels are computed using the non-parametric Games-Howell post hoc test. FIG. 5D: Violin plot showing the expression-based score of exhausted CD8 T cells with their mean scores and p values labeled. Significance levels are computed using the non-parametric Games-Howell post hoc test. FIG. 5E: Scatterplot results from the Pearson correlation analysis of HRH1 and M2-like macrophage markers CD163 (left) and CD209 (right) at the single-cell level in TAMs of human melanomas (GSE115978). FIG. 5F: Scatterplot results from the Pearson correlation analysis of HRH1 and M1-like macrophage markers IDO1 (left) and IRF1 (right) at the single-cell level in TAMs of human melanomas (GSE115978). See also FIGS. 13A-13L.

FIGS. 6A-6G. HRH1 inhibition enhances ICB therapeutic efficacy according to aspects of this disclosure. FIG. 6A: EO771 tumor growth and survival analysis of vehicle- or FEXO-treated WT and PD-L1−/− mice. n=15-23 mice/group for tumor volume analysis; n=9 mice/group for survival analysis; two-way ANOVA for tumor volume comparison; log-rank test for survival comparison. FIG. 6B: Flow cytometry analysis of VISTA+ TAMs and IFN-g+ CD8+ T cells in EO771 tumors collected from WT and HRH1−/− mice receiving IgG or anti-PD-1 antibody treatment (n=6, one-way ANOVA). FIG. 6C: Primary tumor growth (left) and survival analysis (right) of CT26 tumor-bearing WT mice treated with FEXO alone, anti-CTLA-4 alone, or FEXO+anti-CTLA-4. n=9-10 mice/group for tumor volume and survival analysis. FIGS. 6D-6E: Tumor growth (FIG. 6D) and survival analysis (FIG. 6E) of B16-GM tumor-bearing WT mice with the indicated treatment (n=6-10 mice/group). FIG. 6F: Flow cytometry analysis of VISTA+ TAMs and IFN-g+ CD8+ T cells in primary tumor tissues from B16-GM-bearing mice treated with the indicated regimens (n=5, one-way ANOVA). FIG. 6G: Flow cytometry analysis of MHCII:CD206 ratios of TAMs and IFN-g+ CD8+ T cells in B16-GM primary tumor tissues from mice treated with indicated regimens (n=6, one-way ANOVA). Mean±SEM, two-way ANOVA for tumor volume comparison, log-rank test for survival comparison. *p<0.05; **p<0.01; ***p<0.001; ****p<0.0001. See also FIGS. 14A-14J.

FIGS. 7A-7K. HRH1 blockade rescues allergy-induced immunotherapy resistance according to aspects of this disclosure. FIG. 7A: Experimental schematics of EMT6 or CT26 tumor model with concurrent allergy. FIG. 7B: Serum histamine levels detected by ELISA in age-matched healthy mice, allergic mice, and tumor-bearing mice (10 days after EMT6 tumor cell inoculation) with or without induced allergy (n=6, t test). FIG. 7C: EMT6 tumor growth in sham control group, allergy group, and allergy plus FEXO treatment group (n=6 mice per group). FIG. 7D: Flow cytometry analysis of VISTA+ TAMs and IFN-g+ CD8+ T cells in EMT6 tumor tissues from sham control group, allergy induction group, and allergy plus FEXO treatment group (n=5, one-way ANOVA). FIG. 7E: EMT6 tumor growth in mice with or without concurrent allergic disease, treated with vehicle, ICB, or ICB+FEXO (n=6 mice per group). FIG. 7F: CT26 tumor growth in mice with or without concurrent allergic disease, treated with indicated therapies (n=7 mice per group). FIG. 7G: Comparison of deceased patient percentages according to patient allergy status before receiving anti-PD-1/PD-L1 treatment in melanoma and lung cancer patients.

FIG. 7H: Comparison of plasma histamine levels in pre-treatment blood collected from cancer patient groups with different responses to anti-PD-1 treatment (one-way ANOVA). CR, complete response (100% remission); PR, partial response (R30% remission); SD, stable disease (<30% remission to <20% increase of tumor size); PD, progressive disease (R20% increase). FIG. 7I: A waterfall plot depicting the responses to anti-PD-1 treatment in cancer patients with low levels (<0.3 ng/mL), medium levels (>0.3 to <0.6 ng/mL), and high levels (>0.6 ng/mL) of plasma histamine. FIG. 7J: Assessment of the objective response rate (ORR) and disease control rate (DCR) among cancer patients with different plasma histamine levels (low, medium, and high) (Fisher's exact test). FIG. 7K: The distributions of age, sex, and tumor stage among anti-PD-1-treated lung cancer patients (n=48) with indicated histamine level (Fisher's exact test). Mean±SEM, two-way ANOVA for tumor volume comparison. *p<0.05; **p<0.01; ***p<0.001; ****p<0.0001; NS, not significant. See also FIGS. 14A-14J and Tables S3-S6.

FIGS. 8A-8F. Correlations between HRH1 expression and patients' clinical outcomes in CTL+ tumors across multiple cancer types, related to FIG. 1, according to aspects of this disclosure. FIG. 8A: Percentages of deceased cancer patients taking aspirin or penicillin during anti-PD-1/PD-L1 treatment versus those did not. FIG. 8B: The distributions of age-, sex-, and tumor stages among melanoma patients encountered at MD Anderson who took H1-antihistamines during immunotherapy compared with those who did not. FIG. 8C: Percentages of deceased cancer patients taking H1-antihistamines during anti-PD-1/PD-L1 treatment versus those did not (Fisher exact test). (FIG. 8D) Percentages of deceased cancer patients taking H1-antihistamines during chemotherapy treatment versus those did not (Fisher exact test). FIGS. 8E-F: Kaplan-Meier survival analysis of low HRH1 (blue) versus high HRH1 (red) in CTL+ or CTL− lung adenocarcinoma (LUAD) (FIG. 8E) in TCGA datasets. The numbers at risk are the stratified HRH1 level high and low patient numbers of those who remained alive and uncensored after a certain time period. FIG. 8F: Volcano plots of hazard ratios and −log 2 (P values) from the coxph analysis of CTL+ triple-negative breast cancer (TNBC) in TCGA dataset. Black, all 16,975 genes. Red and blue, genes associated with poor and favorable outcomes, respectively. Horizontal dashed line marks a threshold P value of 0.0025.

FIGS. 9A-9J. HRH1 expression in cancer cell lines and macrophages, related to FIG. 2, according to aspects of this disclosure. FIGS. 9A-9B: Flow cytometry analysis of HRH1 expression on human (FIG. 9A) and mouse (FIG. 9B) cancer cell lines. FIGS. 9C-9D: Analysis of correlations between HRH1 expression and tumor purity (FIG. 9C), B-cells, CD8+ T cells, CD4+ T cells, macrophages, and neutrophils (FIG. 9D) in TCGA basal-like breast cancers using Tumor IMmune Estimation Resource (TIMER). The purity-corrected partial Spearman's correlation coefficient and statistical P value were presented. FIG. 9E: Representative western blot analysis of HRH1 expression in human and mouse cancer cell lines, and macrophages. FIG. 9F: Gating strategy to identify HRH1+ subsets in 4T1 tumor. Single cells isolated from digested tumor tissues were gated for viable, hemopoietic cells (CD45+), myeloids (CD45+ CD11b+), and macrophages (CD11b+Grl−F4/80+). FIG. 9G: MFI of HRH1 on naïve, TGF-β (20 ng/ml)-treated, or indicated TCM-treated BMDMs. TCM was collected from scramble ctrl (shctrl) or TGF-β1 knocked down (shTGFB1) 4T1 or EMT6 cells (one-way ANOVA). (FIGS. 9H-9J) Histamine levels in culture medium from the indicated tumor tissues (FIG. 9H), blood plasma (FIG. 9I), and cell lines (FIG. 9J) were detected by ELISA. MEC, mammary epithelial cells (n=3-6, t-test or one-way ANOVA). FIG. 9K: HDC expression in normal human breast tissues and TNBC tissues was detected by INC. Mean±SEM, **P<0.01, ***P<0.001, ****P<0.0001.

FIGS. 10A-10M. HRH1 inhibition enhances T cell anti-tumor activity, related to FIG. 3, according to aspects of this disclosure. (FIG. 10A) Relative mRNA levels of indicated M1 and M2 markers in TAMs of EO771 tumors growing in WT and HRH1−/− mice. (FIG. 10B) Representative histograms and evaluation of CD8+ T cell proliferation based on carboxyfluorescein succinimidyl ester (CFSE) dilution in vitro. CD3/CD28 activated T cells were co-cultured with WT, HRH1−/−, vehicle-or FEXO (10 μM)-treated BMDMs (TCM-treated) for 24 hours before flow cytometry analysis. (FIG. 10C) Representative flow cytometry analysis and quantification of splenic PRF1+ CD8+ T cells activated by CD3/CD28 in vitro. Activated T cells were first co-cultured with WT, HRH1−/−, vehicle- or FEXO (10 j.tM)-treated BMDMs (TCM-treated) for 24 hours before flow cytometry analysis. (FIG. 10D) Estimation of OT1-mediated killing of EO771-OVA cells in the presence of WT BMDM, HRH1−/− BMDM, vehicle-treated or FEXO-treated BMDM (n=6, t-test). (FIG. 10E) Representative flow cytometry analysis and quantification of M1-like macrophage marker (MHCII+) and M2-like macrophage marker (CD206+) expression in vehicle- or histamine (HIS, 10 j.tM)-treated peritoneal macrophages. (FIGS. 10F-10G) Representative flow cytometry analysis and quantification of splenic IFN-γ+CD8+ T cells (FIG. 10F) and PRF1+ CD8+ T cells (FIG. 10G) activated by CD3/CD28 in vitro. Activated T cells were first co-cultured with vehicle- or HIS (10 j.tM)-treated peritoneal macrophages for 24 hours before flow cytometry analysis. (FIG. 10H) Quantitation of tumor-reactive T cell frequency in B16-GM tumors by IFN-γ ELISPOT assay. (FIG. 10I) Relative MHC II:CD206 MFI ratio of tumor-associated macrophages (TAMs) in 4T1 and LLC tumors from vehicle-treated versus FEXO-treated WT mice (n=5-6, t-test). (FIGS. 10J and 10K) Percentages of IFN-γ+ or PRF1+ CD8+ T cells (FIG. 10J), and tumor growth (K) in 4T1 and LLC tumors from vehicle- or FEXO-treated mice (n=5-6, t-test for flow cytometry analysis; n=6 mice per group, two-way ANOVA for tumor growth analysis). (FIG. 10L) B16-GM tumor growth with indicated treatment. CD8+ T cells were depleted by anti-CD8 antibodies (n=6-7 mice/group, two-way ANOVA). (FIG. 10M) Evaluation of tumor blood vessel density by IHC staining of CD31 in EO771 tumors treated with vehicle or FEXO. Top: representative IHC staining slides; bottom: quantification of CD31 IHC staining using H-score (left) or CD31+ cell percentage (right). (FIG. 10N) Comparison of CD8+ T cell proliferation (CFSE dilution) and cytotoxic/cytolytic activities (IFN-γ+ and PRF1+) between WT versus HRH1−/−, and vehicle-versus FEXO (10 j.tM)-treated CD8+ T cells analyzed by flow cytometry (n=5, t-test). Mean±SEM, *P<0.05, **P<0.01, ***P<0.001, ****P<0.0001, NS: not significant.

FIGS. 11A-11C. Bone marrow chimeric mice experiments, macrophage co-implantation and profiling of immune microenvironment of EO771 tumors by CyTOF, related to FIG. 3, according to aspects of this disclosure. (FIG. 11A) Tumor growth, MHCII:CD206 MFI ratio and VISTA expression in TAMs, and IFNγ+ CD8+ T cell infiltration in EO771 tumors from indicated chimeric mice. WT in WT:WT mice reconstituted with WT bone marrow cells; HRH1−/− in WT:WT mice reconstituted with HRH1−/− bone marrows; WT in HRH1−/−: HRH1−/− mice reconstituted with WT bone marrows (n=6 mice/group, one-way ANOVA). (FIG. 11B) Growth of EO771 and LLC tumor cells co-implanted with WT or HRH1−/− BMDMs in WT recipient mice, respectively (n=6 mice/group, two-tailed t-test). (FIG. 11C) Frequency of clusters of indicated immune cell subsets in EO771 tumors from WT (left bars) and HRH1−/− (right bars) mice (t-test). Mean±SEM, *P<0.05,**P<0.01.

FIGS. 12A-12D. HRH1 inhibition enhances T cell anti-tumor activity in lung and inhibits lung metastasis, related to FIG. 3, according to aspects of this disclosure. (FIG. 12A) Gating strategy used to identify resident alveolar macrophage (AM) subset (CD11b-Siglec-F+ CD11C+) in mouse lung tissues. (FIGS. 12B and 12C) Relative MHC II:CD206 MFI ratio of AMs, the percentage of IFN-γ+ CD8+ T cells and lung metastatic nodules in lung tissues from B16-GM tumor-bearing WT versus HRH1−/− mice (FIG. 12B) and 4T1 tumor-bearing mice treated with vehicle or FEXO (FIG. 12C) were analyzed by flow cytometry (n=5-6, t-test). FIG. 12 (FIG. 12D) Quantification of lung metastatic nodules in vehicle- or FEXO-treated mice with surgical removal of B16-GM primary tumors at early stage (n=5, t-test). Mean±SEM, *P<0.05, **P<0.01.

FIGS. 13A-13L. HRH1 activation up-regulates membrane VISTA on macrophages, related to FIGS. 4 and 5, according to aspects of this disclosure. (FIG. 13A) Percentages of splenic PRF1+ CD8+ T cells co-cultured with EO771 TCM-treated WT or HRH1−/− BMDMs in a direct-contact or separately in transwells (n=6, t-test). (FIG. 13B) Representative flow cytometry plots of VISTA and TIM-3 expression on naïve or TCM-treated WT or HRH1−/− BMDMs. (FIG. 13C) Percentages of splenic PRF1+ CD8+ T cells co-cultured with TCM-treated WT or HRH1−/− BMDMs pretreated with anti-TIM-3 (10 j.tg/ml), anti-VISTA (10 j.tg/ml) antibodies, or combination of them (n=3-4, one-way ANOVA). (FIG. 13D) Evaluation of OT1-mediated killing of EO771-OVA cells in the presence of BMDM pretreated with IgG, anti-TIM-3 (10 μg/ml), anti-VISTA (10 μg/ml) antibodies, or combination of them (n=6, one-way ANOVA). (FIG. 13E) Pearson correlation between HRH1 and VISTA expression (MFI) on TAMs from EO771 tumors (n=15). (FIG. 13F) Flow cytometry analysis of VISTA+ TAMs in 4T1 or LLC tumor tissues from mice treated with vehicle versus FEXO (n=6, t-test). (FIG. 13G) Flow cytometry analysis of VISTA+ AMs in lung tissues from mice treated with vehicle versus FEXO (4T1 tumors), or WT versus HRH1−/− mice (B16-GM tumors) (n=6, t-test). (FIG. 13H) Relative mRNA level of VISTA in naïve or TCM-treated WT and HRH1−/− BMDMs (n=6, t-test). (FIG. 13I) Mobilization of intracellular calcium determined by Fluo-Forte calcium assay. The intracellular calcium concentration was indicated by Fluo-Forte fluorescence. HIS (histamine), 1 μM; FEXO, 10 μM; ionomycin-Ca2+, 1 μg/ml; BAPTA-AM, 10 μM. (FIG. 13J) The expression of indicated genes in naïve, TCM-treated WT and HRH1−/− BMDM. (FIG. 13K) Bar chart showing different cellular composition of monocytes/macrophage subsets between HRH1-knockout (KO) (bottom bars) and WT mice (top bars) groups. (FIG. 13L) Scatter-plot results from the Pearson's correlation analysis of HRH1 and indicated M2-like macrophage markers at single-cell level in TAMs of human melanomas (GSE115978). Mean±SEM, all in vitro experiments were performed at least three times. *P<0.05, **P<0.01, ****P<0.0001, NS: not significant.

FIGS. 14A-14J. H1-antihistamines synergize with ICB therapy and rescue cancer cell- and allergy-induced immune suppression, related to FIGS. 6 and 7, according to aspects of this disclosure. (FIG. 14A) EO771 tumor growth and HRH1+/VISTA+ TAMs from partial responders versus non-responders (n=4, t-test). (FIG. 14B) EO771 tumor growth in WT and HRH1−/− mice treated with IgG or anti-PD-1 antibody (n=7-13 mice/group, two-way ANOVA). (FIG. 14C) Lung metastasis of B16-GM tumor-bearing WT mice treated with ICB alone, FEXO alone, or ICB+FEXO (n=6, one-way ANOVA). (FIG. 14D) Flow cytometry analysis of VISTA+ TAMs and IFN-γ+ CD8+ T cells in lung tissues from B16-GM-bearing mice treated with indicated regimens (n=5, one-way ANOVA). (FIG. 14E) Tumor growth in mice that had complete B16-GM tumor remission in FEXO+ICB group followed by B16-GM or EO771 cell re-challenging (4 mice/each group). (FIG. 14F) Quantification of metastatic lung nodules, relative MHC II:CD206 MFI ratios of AMs, and IFN-γ+ CD8+ T cells in lung tissues from mice-bearing B16-GM tumor treated with indicated regimens (n=5-6, one-way ANOVA). (FIG. 14G) Histamine levels detected by ELISA in normal mammary tissue and EMT6 tumors with or without induced allergic reaction (n=3, one-way ANOVA). (FIG. 14H) CT26 tumor growth in allergic or sham control mice, followed by vehicle or FEXO treatment (n=7-8 mice/group, two-way ANOVA for tumor volume comparison). (FIG. 14I) Analysis of relative MHC II:CD206 MFI ratio (TAMs) in EMT6 tumors from sham control mice, allergic mice, and allergic mice treated with FEXO (n=5, t-test). (FIG. 14J) Analysis of VISTA+ TAMs, relative MHC II:CD206 MFI ratio of TAMs and IFN-γ+ CD8+ T cells in CT26 tumors from sham control mice or allergic mice treated with vehicle or FEXO (n=5-6, t-test). Mean±SEM, *P<0.05, **P<0.01, ***P<0.001, ****P<0.0001.

FIGS. 15A-15B. FIG. 15A: Scatterplot of actual numbers (Y-axis) of patients diagnosed with early-stage breast cancer who took various commonly used medicines (50 most prescribed drugs+20 OTC drugs) and had poor outcome (tumor recur or patient deceased) versus the estimated numbers of patients with poor outcomes (estimated 2.54%, based on whole patient population). Each dot represents a group of patients taking one type of medicine. The arrow highlights the patients taking H1-antihistamines. FIG. 15B: Comparison of percentage of patient outcomes (recurrence, deceased, poor outcome) for early-stage breast cancer patients that were treated with H1-antihistamines (left bars) or not treated with H1-antihistamines (right bars). Patients with poor outcomes include patients with cancer recurrence and deceased patients. The numbers noted above the bars are the patient numbers with indicated outcomes (top) and total patient number (bottom).

### DETAILED DESCRIPTION

Provided herein are methods and compositions for determining the likelihood that a subject with cancer will respond to an immunotherapy comprising a checkpoint inhibitor. In particular, the present disclosure involves methods for measuring the level of histamine and/or HRH1 signaling activation in a biological sample from the subject, e.g., directly in a plasma sample or indirectly by quantifying, e.g., histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages in a tumor sample, or by virtue of the presence of allergies or elevated IgE levels in the subject. Responding can involve, inter alia, an increase in overall survival, an increased duration of remission, a decreased likelihood of relapse, or any other measure indicating that the checkpoint inhibitor is acting to reduce, eliminate, slow, attenuate, or otherwise negatively affect the growth, proliferation, and/or survival of cancer or tumor cells in the subject. Subjects that are determined to have a high likelihood of responding to a checkpoint inhibitor (i.e., subjects with a low level of histamine or HRH1 signaling activation) can be directly treated using an immunotherapy comprising the inhibitor, whereas subjects determined to have a low likelihood of responding (i.e., subjects with elevated levels of histamine or HRH1 signaling activation) can be treated using an alternative therapeutic approach or by administering an antihistamine in conjunction with the immunotherapy.

The present disclosure provides methods and compositions for, inter alia, i) assessing response to immunotherapies by measuring histamine and/or HRH1 signaling activation levels by, e.g., detection of HRH1+ macrophages, mast cells, histamine, HDC expression, HRH1 expression, VISTA+ macrophages, PI3K-gamma+ macrophages, and/or TIM-3+ macrophages in plasma or in tumors; ii) determining if a patient should be treated with an H1-antihistamine before receiving immunotherapy; iii) combinatorial treatment of patients with immunotherapies and H1-antihistamines; iv) the identification of more effective H1-antihistamines as an adjuvant of immunotherapies; and v) the identification or development of methods and/or products that block the histamine/HRH1 axis and/or downstream effectors, e.g., by reducing the production of histamine and/or using anti-VISTA antibodies, as adjuvants of immunotherapies.

### A. Subjects and Samples

The present methods and compositions can be used to determine whether a subject with cancer is likely to respond to an immunotherapy comprising the administration of a checkpoint inhibitor. In various embodiments, the subject may be an adult of any age, a child, or an adolescent. The subject may be male or female. In particular embodiments, the subject is a human.

As used herein, the term “cancer” is intended to include any member of a class of diseases characterized by the uncontrolled growth of aberrant cells. The term includes all known cancers and neoplastic conditions, whether characterized as malignant, benign, recurrent, soft tissue, or solid, and cancers of all stages and grades including advanced, recurrent, pre- and post-metastatic cancers. In particular embodiments, the cancer comprises tumors or malignant cells that produce high levels of histamine and/or result in high plasma histamine levels in a subject, and/or tumors that show elevated HRH1 signaling activity, including, but not limited to, breast cancer, colorectal cancer, lung cancer, and melanoma. Examples of different types of cancer that can be treated using the present methods include, but are not limited to, prostate cancer (e.g., prostate adenocarcinoma); breast cancers (e.g., triple-negative breast cancer, ductal carcinoma in situ, invasive ductal carcinoma, tubular carcinoma, medullary carcinoma, mucinous carcinoma, papillary carcinoma, cribriform carcinoma, invasive lobular carcinoma, inflammatory breast cancer, lobular carcinoma in situ, Paget's disease, Phyllodes tumors); gynecological cancers (e.g., ovarian, cervical, uterine, vaginal, and vulvar cancers); lung cancers (e.g., non-small cell lung cancer, small cell lung cancer, mesothelioma, carcinoid tumors, lung adenocarcinoma); digestive and gastrointestinal cancers such as gastric cancer (e.g., stomach cancer), colorectal cancer, gastrointestinal stromal tumors (GIST), gastrointestinal carcinoid tumors, colon cancer, rectal cancer, anal cancer, bile duct cancer, small intestine cancer, and esophageal cancer; thyroid cancer; gallbladder cancer; liver cancer; pancreatic cancer; appendix cancer; renal cancer (e.g., renal cell carcinoma); cancer of the central nervous system (e.g., glioblastoma, neuroblastoma); skin cancer (e.g., melanoma); bone and soft tissue sarcomas (e.g., Ewing's sarcoma); lymphomas; choriocarcinomas; urinary cancers (e.g., urothelial bladder cancer); head and neck cancers; and bone marrow and blood cancers (e.g., chronic lymphocytic leukemia, lymphoma). As used herein, a “tumor” comprises one or more cancerous cells.

The subject may have one or more symptoms of cancer. A non-limiting list of possible symptoms includes loss of appetite, weight loss, fatigue, weakness, eating problems, swelling or lumps in the body, pain, skin changes, cough, hoarseness, bruising, change in bowel habits, bladder changes, fever, night sweats, feeling cold, dizzy, or lightheaded, headaches, pale skin, shortness of breath, bruises or other red or purple spots on the skin, excess bleeding, nosebleeds, bleeding gums, weakness in one side of the body, slurred speech, confusion, sleepiness, blurry vision, loss of vision, hearing problems, sores, chest pain, bone pain, joint pain, swelling in the abdomen, nausea, facial numbness, loss of balance, seizures, enlarged lymph nodes, anemia, leukopenia, neutropenia, thrombocytopenia, and others. The symptoms can be mild, moderate, or severe. A diagnosis of cancer can be based on, e.g., medical history, physical exam, or lab test, e.g., as performed on a blood sample, bone marrow aspiration or biopsy, or cerebrospinal fluid sample. Lab tests can comprise, e.g., blood cell counts, coagulation tests, cell analysis by microscope, cytochemical analysis, flow cytometry, immunohistochemistry, cytogenetic test, fluorescence in situ hybridization (FISH), polymerase chain reaction (PCR) assays, imaging tests such as X-rays, computerized tomography (CT) scan, positron emission tomography (PET) scan, magnetic resonance imaging (MRI), ultrasound, and others.

A “response” to an immunotherapy comprising a checkpoint inhibitor can refer to any lasting, detectable improvement in any symptom of the cancer (e.g., any symptom as described elsewhere herein) in the subject. A patient or subject showing a response to an immunotherapy means that the patient is a “responder” or is “responsive” to the treatment. In particular embodiments, a determination that a patient is a “responder” to a checkpoint inhibitor means that the patient shows a detectable improvement in one or more symptom or feature of the cancer, e.g., tumor size, survival, remission duration, likelihood of relapse, or any of the symptoms listed above.

In some embodiments, the present methods comprise administering immunotherapy to the subject. In some embodiments, the immunotherapy comprises administering an immune checkpoint blockade binding agent to the subject. In some embodiments, the immune checkpoint blockade binding agent is antibody comprising one or more of an anti-CTLA4 antibody, an anti-PD1 antibody, an anti-PD-L1 antibody, an anti-LAG-3 antibody, an anti-TIM-3 antibody, an anti-TIGIT antibody, an anti-CD47 antibody, or an anti-VISTA antibody. In some embodiments, the antibody is a human antibody, chimeric antibody, humanized antibody, an F(ab)′2, an Fab, an Fv, a single domain antibody, a bispecific antibody, a helix-stabilized antibody, a single-chain antibody molecule, a disulfide stabilized antibody, or a domain antibody. In some embodiments, the immunotherapy comprises administering at least one of a chimeric antigen receptor (CAR)-T cell, a CAR-natural killer (NK) cell, a CAR-macrophage, a tumor vaccine, an oncolytic virus vaccine, a vaccine against an infectious agent, a co-stimulatory mAb, an epigenetic modulator, a TLR3/7/8/9 agonist, an anti-CD47 antibody, and/or IL-2 receptor agonist to the subject.

In some embodiments, the subject is treated with one or more additional or alternative therapies for the cancer (i.e., in addition to or in place of the herein-described immunotherapies), e.g., in subjects determined to have a low likelihood of responding to a checkpoint inhibitor). Such additional or alternative therapies can include, e.g., chemotherapy, targeted therapies (e.g., monoclonal antibodies, hedgehog pathway inhibitors), non-chemotherapy drugs such as all-trans retinoic acid or arsenic trioxide, surgery, radiation, stem cell transplants, and others.

To assess the “immune checkpoint responder status” of the subject (i.e., whether the subject has a high or low likelihood of responding to the inhibitor), a biological sample is obtained from the subject. In some embodiments, the biological sample is a blood sample, such as serum or whole blood. In some embodiments, the biological sample is plasma. In some embodiments, the biological sample is a tumor sample, e.g., a bone marrow aspirate or biopsy. Generally, any sample that comprises histamine or histamine-responsive cells (e.g., HRH1+ macrophages, particularly HRH1+ macrophages from a tumor sample) can be used. Other suitable samples include urine, ascites, seminal fluid, vaginal secretions, cerebrospinal fluid (CSF), synovial fluid, pleural fluid (pleural lavage), pericardial fluid, peritoneal fluid, amniotic fluid, saliva, nasal fluid, otic fluid, gastric fluid, breast milk, amniotic fluid, bile, gastric juice, lymph, mucus, pericardial fluid, peritoneal fluid, pleural fluid, pus, saliva, sebum, serous fluid, sputum, sweat, tears, and others. The sample can be obtained from the subject using conventional techniques known in the art.

### B. Detecting Histamine and HRH1 Signaling Levels

The levels of histamine or HRH1 signaling activation in the sample can be assessed in any of a number of ways. As used herein, the “levels of histamine” refer not only to the amount of histamine in, e.g., plasma or a tumor sample, but also to other indices of histamine activity or production and/or HRH1 signaling activation, e.g., HRH1 expression and/or the number of HRH1+ macrophages, HDC expression, mast cells, VISTA+ macrophages, PI3K-gamma+ macrophages, TIM-3+ macrophages in a tumor sample, or indirect aspects such as the presence of allergies or elevated plasma IgE levels in an individual.

Histamine (PubChem CID 774) refers to a imidazole that is 1H-imidazole substituted at position C-4 by a 2-aminoethyl group. Histamine acts through receptors H1, H2, H3, and H4. When activated, histamine receptor H1 (HRH1; NCBI Gene ID: 3269; UniProt ID P35367) signaling can activate various downstream effectors, including VISTA (V-set immunoregulatory receptor, VSIR; NCBI Gene ID: 64115, UniProt ID Q9H7M9), PI3K-gamma (phosphatidylinositol-4,5-bisphosphate 3-kinase catalytic subunit gamma, NCBI Gene ID 5294, UniProt ID P48736), and TIM-3 (hepatitis A virus cellular receptor 2, or HAVCR2; NCBI Gene ID 84868, UniProt ID Q8TDQO). The major histamine-producing enzyme in mammals is histamine decarboxylase (HDC, NCBI Gene ID 3067, UniProt ID P19113).

In some embodiments, the histamine levels are assessed in blood samples. In particular embodiments, histamine levels are detected in plasma, which is prepared by collecting blood using a tube with an anticoagulant such as EDTA, sodium heparin, or sodium citrate, and removing the cells by centrifugation. The resulting supernatant, or plasma, is then used for the detection of histamine levels, e.g., by ELISA. In some embodiments, the histamine levels or indices or histamine levels or HRH1 signaling activity are assessed in tumor samples, e.g., as obtained from a biopsy. In some such embodiments, histamine levels or HRH1 signaling activity are detected by examining the level of, e.g., HRH1 expression, HDC expression, HRH1+ macrophages, VISTA+ macrophages, PI3K-gamma+ macrophages, TIM-3+ macrophages, or mast cells in a tumor sample, e.g., by detecting HRH1 or HDC protein levels by ELISA or IHC or by detecting HRH1 or HDC mRNA levels using, e.g., qRT-PCR. In some embodiments, the number of HRH1+, VISTA+, PI3K-gamma+, and/or TIM-3+ macrophages in a tumor sample is detected, e.g., using immunohistochemistry (IHC) and/or fluorescence activated cell sorting (FACS). In some embodiments, histamine levels are determined indirectly, e.g., by virtue of the subject presenting or having a history of allergies, e.g., an allergy to an inhalant allergen (ragweed pollen, dust mite, pet dander), drugs (penicillin, sulfa, aspirin), stinging insect venoms, foods (egg, wheat, milk, fish), etc., and or having an elevated level of plasma IgE.

The measurement of histamine levels, e.g., in serum, can be assessed using routine techniques such as immunoassays and quantitative mass spectrometry that are known to those skilled in the art. In particular embodiments, the histamine level is determined using an enzyme-linked immunosorbent assay (ELISA) assay. In some embodiments, a histamine ELISA kit is used, e.g., as available from Enzo Life Sciences (Cat. #ENZ-KIT-140-0001). Such assays use antibodies directed against histamine, e.g., antibody ab213975 (Abcam).

Levels of histamine and of proteins, e.g., HRH1 or HDC, can be assessed using routine techniques such as immunoassays, immunohistochemistry, western blotting, two-dimensional gel electrophoresis, and quantitative mass spectrometry that are known to those skilled in the art. Protein quantification techniques are generally described in “Strategies for Protein Quantitation,” Principles of Proteomics, 2nd Edition, R. Twyman, ed., Garland Science, 2013. In some embodiments, protein expression or stability is detected by immunoassay, such as but not limited to enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); immunofluorescence (IF); fluorescence polarization immunoassays (FPIA); and chemiluminescence assays (CL). If desired, such immunoassays can be automated. Immunoassays can also be used in conjunction with laser induced fluorescence (see, e.g., Schmalzing et al., Electrophoresis, 18:2184-93 (1997); Bao, J. Chromatogr. B. Biomed. Sci., 699:463-80 (1997)).

Gene expression levels (e.g., HRH1) can also be detected by quantifying mRNA levels, e.g., in macrophages in the tumor environment. mRNA levels can be analyzed using routine techniques such as RT-PCR, Real-Time RT-PCR, semi-quantitative RT-PCR, quantitative polymerase chain reaction (qPCR), quantitative RT-PCR (qRT-PCR), multiplexed branched DNA (bDNA) assay, microarray hybridization, or sequence analysis (e.g., RNA sequencing (“RNA-Seq”)). Methods of quantifying polynucleotide expression are described, e.g., in Fassbinder-Orth, Integrative and Comparative Biology, 2014, 54:396-406; Thellin et al., Biotechnology Advances, 2009, 27:323-333; and Zheng et al., Clinical Chemistry, 2006, 52:7 (doi: 10/1373/clinchem.2005.065078). In some embodiments, real-time or quantitative PCR or RT-PCR is used to measure the level of a polynucleotide (e.g., mRNA) in a biological sample. See, e.g., Nolan et al., Nat. Protoc, 2006, 1:1559-1582; Wong et al., BioTechniques, 2005, 39:75-75. Quantitative PCR and RT-PCR assays for measuring gene expression are also commercially available (e.g., TaqMan® Gene Expression Assays, ThermoFisher Scientific).

In some embodiments, the presence of HRH1+ macrophages, VISTA+ macrophages, PI3K-gamma+ macrophages, or TIM-3+ macrophages in the tumor environment is detected. In particular embodiments, HRH1+, VISTA+, PI3K-gamma+, and/or TIM-3+ macrophages are detected and analyzed by FACS. For example, in some embodiments, a tumor sample obtained from the subject can be processed to produce a single-cell suspension (e.g., cut into pieces with scissors, subjected to enzymatic digestion, and then filtered). Following lysis of red blood cells, the samples can be washed and resuspended in, e.g., flow cytometry buffer. Flow cytometry staining and analysis can then be performed, e.g., as described in the Examples. In particular embodiments, an antibody against HRH1 is used, e.g., antibody 480054 from R&D Systems or antibody AHR-001 from Alomone labs. In particular embodiments, an antibody against VISTA is used, e.g., antibody MH5A from BioLegend. In particular embodiments, an antibody against TIM-3 is used, e.g., antibody B8.2C12 from BioLegend.

In some embodiments, replicates (e.g., duplicates, triplicates) of any of the herein-described assays may be run for each sample in order to gain a higher level of confidence in the data. Replicate values can be averaged, and standard deviations can be calculated.

In general, the measured histamine level or marker of HRH1 signaling activation (e.g., HDC mRNA, HDC protein, mast cells, HRH1 mRNA, HRH1 protein, HRH1+ macrophages, VISTA+ macrophages, PI3K-gamma+, and/or TIM-3+ macrophages) is compared to a reference or control level, e.g., a level of histamine, expression, or cells obtained from or representative of an individual with average or low levels of serum histamine and/or who is known to be responsive to immunotherapy with a checkpoint inhibitor. In some embodiments, the measured level of histamine or marker of HRH1 signaling activation is compared to an internal control, e.g., a compound in the serum or cells that is known to not vary substantially between individuals (e.g., a housekeeping protein). In some embodiments, the measured histamine level or marker of HRH1 signaling activation in the subject is at least about 10%, 15%, 20%, 25%, 30%, 35%, 40%, 45%, 50%, 55%, 60%, 65%, 70%, 75%, 80%, 85%, 90%, or more higher than the control or reference level. In some instances, the measured histamine level or marker of HRH1 signaling activation level in the subject can be compared to a positive control who is an individual known not to be responsive to immunotherapy with a checkpoint inhibitor

In some embodiments, the detection is carried out in whole or in part using an integrated system, as described elsewhere herein, which may also comprise a computer system as described elsewhere herein.

### C. Determining Checkpoint Inhibitor Responder Status

In one aspect, provided herein is a method of determining the likelihood of responding to a checkpoint inhibitor in a subject with cancer comprising, consisting essentially of, or consisting of: detecting differential levels of histamine and/or HRH1 signaling activation as disclosed herein in a biological sample, such as a plasma sample or tumor biopsy obtained from the subject, as compared to a control.

In some embodiments, the level of histamine and/or HRH1 signaling activation is quantified and compared to one or more preselected or threshold levels. Threshold values can be selected that provide an ability to predict the likelihood of responding to a checkpoint inhibitor. Such threshold values can be established, e.g., by calculating receiver operating characteristic (ROC) curves using a first cancer patient population that responds to a checkpoint inhibitor and a second cancer patient population that does not respond to a checkpoint inhibitor.

In some embodiments, a classifier is generated (also referred to as training) for use in the methods of determining the likelihood of responding to a checkpoint inhibitor in a subject with cancer. As used herein, the terms “classifier” and “predictor” are used interchangeably and refer to a mathematical function that uses the values of the signature (e.g. protein levels from a defined set of biomarkers) and a pre-determined coefficient for each signature component to generate scores for a given observation or individual patient for the purpose of assignment to a category. A classifier is linear if scores are a function of summed signature values weighted by a set of coefficients. Furthermore, a classifier is probabilistic if the function of signature values generates a probability, a value between 0 and 1.0 (or 0 and 100%) quantifying the likelihood that a subject or observation belongs to a particular category or will have a particular outcome, respectively. Probit regression and logistic regression are examples of probabilistic linear classifiers.

A classifier, including a linear classifier, may be obtained by a procedure known as training, which consists of using a set of data containing observations with known category membership (e.g., subjects responding or not responding to a checkpoint inhibitor). Specifically, training seeks to find the optimal coefficient for each component of a given signature, where the optimal result is determined by the highest classification accuracy. In some embodiments, a unique classifier may be developed and trained with respect to a particular platform upon which the signature is measured.

Determining the accuracy of classification may involve the use of accuracy measures such as sensitivity, specificity, positive predictive value (PPV), negative predictive value (NPV), accuracy, and area under the curve (AUC) of a Receiver Operating Characteristic (ROC) curve corresponding to the diagnostic accuracy of detecting or predicting checkpoint responsiveness.

In some embodiments, these values may be reported relative to a reference range that indicates the confidence with which the classification is made. In some embodiments, the output of the classifier may be compared to a threshold value, for example, to report a “positive” in the case that the classifier score or probability exceeds the threshold indicating a high likelihood of responding to a checkpoint inhibitor. If the classifier score or probability fails to reach the threshold, the result would be reported as “negative” for the respective condition.

In some embodiments, a subject is determined to have a significant probability of having or not having a specified condition or outcome (e.g., responding to a checkpoint inhibitor). By “significant probability” is meant that the subject has a reasonable probability (0.6, 0.7, 0.8, 0.9 or more) of having, or not having, a specified condition or outcome.

In some embodiments, a determination of a checkpoint responder status in a cancer patient can be based not solely on histamine and/or HRH1 signaling activation levels, but can also take into account clinical and/or other data about the subject, e.g., clinical data about the subject's current medical state, the medical history of the subject, demographic data about the subject (age, sex, etc.), or the results of one or more laboratory tests.

### D. Treatment Decisions

The determination of a likelihood of responding to a checkpoint inhibitor in a cancer patient using the present methods and compositions can be used to inform the delivery of medical care appropriate for the cancer in the subject. For example, a determination that a subject has a high likelihood of responding to a checkpoint inhibitor can lead to a decision to continue or initiate a checkpoint inhibitor therapy in a subject. Alternatively, a determination that a subject has a low likelihood of responding to a checkpoint inhibitor can lead to a decision to initiate an alternative therapy for the patient, or to administer an antihistamine in conjunction with the checkpoint inhibitor. It will be appreciated that the administration of an antihistamine and/or immunotherapy does not preclude the administration of any other anti-cancer treatment, e.g., chemotherapy, radiation, surgery, targeted drug therapy, etc.

In particular embodiments, the antihistamine is an H1 antihistamine, i.e., a compound that blocks the action of histamine via the H1 histamine receptor (H1 receptor). Any such antihistamine can be used, e.g., Cetirizine, Loratadine, Ketotifen, Rupatadine, Bilastine, Terfenadine, Astemizole, Mizolastine, Acrivastine, Ebastine, Bepotastine, Azelastine, Levocabastine, Olopatadine, Levocetirizine, Desloratadine, Fexofenadine, Quifenadine, and derivatives thereof, including but not limited to forms for intravenous injection such as Quzytttir (TerSera Therapeutics).

Any other method of inhibiting H1 histamine activity or levels in the subject can be used. For example, in some embodiments, the antihistamine is a compound that inhibits the production of histamine, e.g., by inhibiting the histidine decarboxylase (HDC) enzyme. In some embodiments, a compound that downregulates a downstream effector of HRH1 signaling activation is used, e.g., an inhibitor of VISTA, PI3K-gamma, or TIM-3, e.g., an anti-VISTA, anti-PI3K-gamma, or anti-TIM-3 antibody.

As described above, the present methods involve the administration of immunotherapies that involve checkpoint inhibitors. An important part of the immune system is its ability to keep itself from attacking normal cells in the body. To do this, it uses “checkpoints”—proteins on immune cells that need to be switched on or off to start an immune response. Cancer cells sometimes use these checkpoints to avoid being attacked by the immune system. The immunotherapy described herein particularly involves immune checkpoint blockade (ICB) therapies, e.g., therapy to disrupt T cell inhibitory signals such as cytotoxic lymphocyte antigen-4 (CTLA-4), programmed cell death protein 1 (PD-1, or programmed cell death ligand 1 (PD-L1). As such, in particular embodiments, the immunotherapy comprises an immune checkpoint blockade binding agent (or “checkpoint inhibitor”) such as an anti-CTLA4 antibody, anti-PD1 antibody, anti-PD-L1 antibody, anti-LAG-3 antibody, anti-TIM-3 antibody, anti-TIGIT antibody, anti-CD47 antibody, or anti-VISTA antibody.

In some embodiments, the subject also receives surgical treatment for the cancer. For example, the patient may receive surgical resection (removal of the tumor with surgery). Small tumors may also be treated with other types of treatment such as ablation or radiation. Ablation is treatment that destroys tumors without removing them. These techniques can be used in patients with a few small tumors and when surgery is not a good option. They are less likely to cure the cancer than surgery, but they can still be very helpful for some people. Ablation is best used for tumors no larger than 3 cm across. For slightly larger tumors (1 to 2 inches, or 3 to 5 cm across), it may be used along with embolization. Because ablation often destroys some of the normal tissue around the tumor, it might not be a good choice for treating tumors near major blood vessels, the diaphragm, or major bile ducts. In some embodiments, the ablation is radiofrequency ablation (RFA). In some embodiments, the ablation is microwave ablation (MWA). In some embodiments, the ablation is cryoablation (cryotherapy). In some embodiments, the ablation is ethanol (alcohol) ablation, e.g., percutaneous ethanol injection (PEI).

In some embodiments, a patient with cancer is also treated using radiation therapy. Radiation therapy uses high-energy rays, or particles to destroy cancer cells. Radiation can be helpful, e.g., in treating cancer that cannot be removed by surgery, cancer that cannot be treated with ablation or did not respond well to such treatment; cancer that has spread to areas such as the brain or bones; patients experiencing severe pain due to large cancers; and patients having a tumor thrombus.

In some embodiments, a patient with cancer is also treated using drug therapy, e.g., targeted drug therapy or chemotherapy. Targeted drugs work differently from standard chemotherapy drugs and include, e.g., kinase inhibitors; Sorafenib (Nexavar), lenvatinib (Lenvima), Regorafenib (Stivarga), and cabozantinib (Cabometyx). Immunotherapy can comprise the administration of monoclonal antibodies. Monoclonal antibodies are designed to attach to a specific target. The monoclonal antibodies used to treat liver cancer affect a tumor's ability to form new blood vessels, also known as angiogenesis. These therapeutics are often referred to angiogenesis inhibitors and include: Bevacizumab (Avastin), which can be used in conjunction with the immunotherapy drug atezolizumab (Tecentriq); Ramucirumab (Cyramza).

Common chemotherapy drugs for treating cancer include, for example: Gemcitabine (Gemzar); Oxaliplatin (Eloxatin); Cisplatin; Doxorubicin (pegylated liposomal doxorubicin); 5-fluorouracil (5-FU); Capecitabine (Xeloda); Mitoxantrone (Novantrone), or combinations thereof. Chemotherapy can be regional when drugs are inserted into an artery that leads to the part of the body with the tumor. thereby focusing the chemotherapy on the cancer cells in that area of the body and reducing side effects by limiting the amount of drug reaching the rest of the body. For example, hepatic artery infusion (HAI), or chemo given directly into the hepatic artery, is an example of a regional chemotherapy that can be used for liver cancer.

Thus, in one aspect, provided herein is a method for treating cancer in a subject comprising, consisting essentially of, or consisting of: administering an effective amount of an checkpoint inhibitor-comprising immunotherapy and an antihistamine to a subject identified as having elevated histamine levels as compared to a control.

In another aspect, provided herein is a method of treating cancer in a subject, the method comprising administering to the subject a therapeutically effective amount of an antihistamine and a checkpoint inhibitor, wherein the subject has been identified as a likely responder to the checkpoint inhibitor based on a detection of elevated levels of plasma histamine or of elevated a marker of HRH1 signaling activation (e.g., histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages) in a tumor sample obtained from the subject as compared to levels in a biological sample from a individual known to be responsive to the immunotherapy.

In some embodiments, the method comprises: providing a biological sample from a subject with cancer; detecting the histamine level in the sample, and comparing the level to a control. In some embodiments, the method comprises determining a checkpoint inhibitor response status in the subject according to the methods described herein.

In some embodiments, a patient receiving a checkpoint inhibitor therapy (e.g., in an indentified responder) or not receiving a checkpoint inhibitor therapy (e.g., in an identified non-responder) is treated with one or more additional or alternative therapies. For example, in some embodiments, the subject is treated with chemotherapy, a targeted therapy (e.g., FLT3 inhibitors, IDH inhibitors, monoclonal antibodies such as gemtuzumab oxogamicin, hedgehog pathway inhibitors), a non-chemotherapy drug such as all-trans retinoic acid or arsenic trioxide, surgery, radiation, stem cell transplants, and others. In particular embodiments, a subject receiving a checkpoint inhibitor is also treated with a chemotherapeutic agent such as cytarabine (ara-C), particularly low-dose cytarabine, and/or a hypomethylating agent (HMA) such as decitabine or azacitidine.

### E. Pharmaceutical Compositions

In some embodiments, the herein-described compounds, e.g., antihistamine and/or immunotherapy checkpoint inhibitor, are present within a pharmaceutical composition or formulation. The pharmaceutical compositions of the compounds of the present disclosure may comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present disclosure (see, e.g., REMINGTON'S PHARMACEUTICAL SCIENCES, 18TH ED., Mack Publishing Co., Easton, PA (1990)).

As used herein, “pharmaceutically acceptable carrier” comprises any of standard pharmaceutically accepted carriers known to those of ordinary skill in the art in formulating pharmaceutical compositions. Thus, the compounds, by themselves, such as being present as pharmaceutically acceptable salts, or as conjugates, may be prepared as formulations in pharmaceutically acceptable diluents; for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

The pharmaceutical compositions will often further comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions of the present disclosure can be administered in a manner compatible with the dosage formulation, and in such amount as will be therapeutically effective. The quantity to be administered depends on a variety of factors including, e.g., the age, body weight, physical activity, hereditary characteristics, general health, sex and diet of the individual, the condition or disease to be treated, the mode and time of administration, rate of excretion, drug combination, the stage or severity of the condition or disease, etc. In certain embodiments, the size of the dose may also be determined by the existence, nature, and extent of any adverse side effects that accompany the administration of a therapeutic agent(s) in a particular individual.

In certain embodiments, the dose of the compound may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of a therapeutic agent calculated to produce the desired onset, tolerability, and/or therapeutic effects, in association with a suitable pharmaceutical excipient (e.g., an ampoule). In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the therapeutic compound.

Methods for preparing such dosage forms are known to those skilled in the art (see, e.g., REMINGTON'S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g., REMINGTON'S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

For oral administration, the therapeutically effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The therapeutically effective dose can also be provided in a lyophilized form. Such dosage forms may include a buffer, e.g., bicarbonate, for reconstitution prior to administration, or the buffer may be included in the lyophilized dosage form for reconstitution with, e.g., water. The lyophilized dosage form may further comprise a suitable vasoconstrictor, e.g., epinephrine. The lyophilized dosage form can be provided in a syringe, optionally packaged in combination with the buffer for reconstitution, such that the reconstituted dosage form can be immediately administered to an individual.

### F. Kits and Systems

### 1. Kits

In one aspect, kits are provided for the determination of the likelihood of a subject with cancer responding to a checkpoint inhibitor, wherein the kits can be used to detect histamine or HRH1 signaling activation in a biological sample obtained from the subject as described herein. The kit may include, e.g., one or more agents for the detection of histamine or HRH1 signaling activation, a container for holding a biological sample, e.g., blood or plasma sample, or biopsy, isolated from a subject with cancer; and instructions for reacting agents with the biological sample or a portion of the biological sample to detect the presence or amount of histamine (or HRH1 signaling activation) in the biological sample. The agents may be packaged in separate containers.

The kit may further comprise one or more control reference samples and reagents for performing the herein-described methods. The kit may also comprise one or more devices or implements for carrying out any of the herein methods.

The kit can comprise one or more containers for compositions contained in the kit. Compositions can be in liquid form or can be lyophilized. Suitable containers for the compositions include, for example, bottles, vials, syringes, and test tubes. Containers can be formed from a variety of materials, including glass or plastic. The kit can also comprise a package insert containing instructions for methods of determining the checkpoint inhibitor response status of a subject with cancer as described above.

### 2. Measurement Systems and Reports for Detecting and Recording Histamine Levels

In one aspect, a system, e.g., measurement system is provided. Such systems allow, e.g., the detection of histamine or HRH1 signaling activation levels in a sample (e.g., plasma histamine levels, or levels of histamine, HDC or HRH1 expression, levels of mast cells, or levels of HRH1+, VISTA+, PI3K-gamma+, or TIM-3+ macrophages in a tumor sample) and the recording of the data resulting from the detection. The stored data can then be analyzed to determine the checkpoint inhibitor response status of a subject such as described above. Such systems can comprise, e.g., assay systems (e.g., comprising an assay device and detector), which can transmit data to a logic system (such as a computer or other system or device for capturing, transforming, analyzing, or otherwise processing data from the detector). The logic system can have any one or more of multiple functions, including controlling elements of the overall system such as the assay system, sending data or other information to a storage device or external memory, and/or issuing commands to a treatment device.

Also provided is a system for detecting histamine or HRH1 signaling activation levels in a sample, by utilizing a station for analyzing the sample by, e.g., ELISA assay or IHC; wherein the sample is a biological sample, e.g., plasma or tumor sample, obtained from a subject with cancer, and the report is useful for determining the likelihood that the subject will respond to an immunotherapy comprising a checkpoint inhibitor. Optionally, a station for generating a report containing information on results of the analyzing is further included.

Also provided is a method of generating a report containing information on results of the detection of histamine or HRH1 signaling activation levels in a sample, including detecting histamine or HRH1 signaling activation in the sample, and generating the report; the sample is a biological sample, e.g., plasma or tumor sample, obtained from a subject with cancer, and the report is useful for determining the likelihood that the subject will respond to an immunotherapy comprising a checkpoint inhibitor.

### 3. Computer Diagnostic Systems for Determining Checkpoint Inhibitor Responder Status

Certain aspects of the herein-described methods may be totally or partially performed with a computer system including one or more processors, which can be configured to perform the steps. Thus, embodiments are directed to computer systems configured to perform the steps of methods described herein, potentially with different components performing a respective step or a respective group of steps. The computer systems of the present disclosure can be part of a measuring system as described above, or can be independent of any measuring systems. In some embodiments, the present disclosure provides a computer system that uses inputted histamine level (and optionally other) data, and determines the checkpoint inhibitor responder status of a subject.

A computer system can include desktop and laptop computers, tablets, mobile phones and other mobile devices. The system can include various elements such as a printer, keyboard, storage device(s), monitor (e.g., a display screen, such as an LED), peripherals, devices to connect a computer system to a wide area network such as the Internet, a mouse input device, scanner, a storage device(s), computer readable medium, camera, microphone, accelerometer, and the like. Any of the data mentioned herein can be output from one component to another component and can be output to the user.

In one aspect, the present disclosure provides a computer implemented method for determining the likelihood of responding to a checkpoint inhibitor in a patient with cancer. The computer performs steps comprising, for example: receiving inputted patient data comprising values for histamine levels in a biological sample from the patient (e.g., determined as described above); analyzing the histamine levels and optionally comparing them to respective reference values, optionally comparing the histamine levels to one or more threshold values to determine checkpoint inhibitor responder status (e.g., determined as described above); and displaying information regarding the responder status or probability in the patient.

In a further aspect, a diagnostic system is included for performing the computer implemented method, as described. A diagnostic system may include a computer containing a processor, a storage component (i.e., memory), a display component, and other components typically present in general purpose computers. The storage component stores information accessible by the processor, including instructions that may be executed by the processor and data that may be retrieved, manipulated or stored by the processor.

The storage component includes instructions for determining the checkpoint inhibitor responder status of the subject. For example, the storage component includes instructions for determining responder status based on histamine or HRH1 signaling activation levels, as described herein. The computer processor is coupled to the storage component and configured to execute the instructions stored in the storage component in order to receive patient data and analyze patient data according to one or more algorithms. The display component displays information regarding the diagnosis of the patient. The storage component may be of any type capable of storing information accessible by the processor, such as a hard-drive, memory card, ROM, RAM, DVD, CD-ROM, USB Flash drive, write-capable, and read-only memories.

The instructions may be any set of instructions to be executed directly (such as machine code) or indirectly (such as scripts) by the processor. In that regard, the terms “instructions,” “steps” and “programs” may be used interchangeably herein. The instructions may be stored in object code form for direct processing by the processor, or in any other computer language including scripts or collections of independent source code modules that are interpreted on demand or compiled in advance.

Data may be retrieved, stored or modified by the processor in accordance with the instructions. For instance, although the diagnostic system is not limited by any particular data structure, the data may be stored in computer registers, in a relational database as a table having a plurality of different fields and records, XML documents, or flat files. The data may also be formatted in any computer-readable format such as, but not limited to, binary values, ASCII or Unicode. Moreover, the data may comprise any information sufficient to identify the relevant information, such as numbers, descriptive text, proprietary codes, pointers, references to data stored in other memories (including other network locations) or information which is used by a function to calculate the relevant data. In certain embodiments, the processor and storage component may comprise multiple processors and storage components that may or may not be stored within the same physical housing. For example, some of the instructions and data may be stored on removable CD-ROM and others within a read-only computer chip. Some or all of the instructions and data may be stored in a location physically remote from, yet still accessible by, the processor. Similarly, the processor may actually comprise a collection of processors which may or may not operate in parallel. In one aspect, computer is a server communicating with one or more client computers. Each client computer may be configured similarly to the server, with a processor, storage component and instructions. Although the client computers and may comprise a full-sized personal computer, many aspects of the system and method are particularly advantageous when used in connection with mobile devices capable of wirelessly exchanging data with a server over a network such as the Internet.

### EXAMPLES

The following examples are offered to illustrate, but not to limit, the claimed invention. Additional examples and figures can be found in Li et al. The allergy mediator histamine confers resistance to immunotherapy in cancer patients via activation of the macrophage histamine receptor H1. Cancer Cell. 2022 Jan. 10; 40(1):36-52.e9; doi: 10.1016/j.ccell.2021.11.002; Epub Nov. 24, 2021, which is incorporated herein in its entirety for all purposes.

### Example 1. Materials and Methods

### Experimental Model and Subject Details

Mice. C57BL/6, BALB/c, and C57BL/6-background HRH1-knockout mice were purchased from The Jackson Laboratory. C57BL/6-back-ground PD-L1-knockout mice were obtained and maintained as previously described (Chen et al., 2014). All mouse protocols and experiments were performed in accordance with National Institutes of Health guidelines and were approved by the MD Anderson Institutional Animal Care and Use Committee. All mice used in our experiments were between 6 and 8 weeks of age, and were housed under standard housing conditions at the MDACC animal facilities. Both male and female C57BL/6 or BALB/c mice were used for lung cancer model (LLC model), melanoma model (B16-GM model) and colon cancer model (CT26), and female BALB/c or C57BL/6 mice were used for breast cancer models (4T1, EMT6 and EO771 models). Animal numbers of each group were calculated by power analysis and animals are grouped randomly for each experiment.

Cell lines. Human mammary tumor cell lines including HCC1806, HS578T, BT-20, MDA-MB-435, MDA-MB-436, MDA-MB-231 and BT-549, murine mammary tumor cell lines including 4T1, EMT6 and EO771, murine lung carcinoma cell line LLC1 (or LLC), colon cancer cell line CT26, L929 cell line and 293T cell line were obtained from American Type Culture Collection (ATCC) and cultured in endo-toxin-free DMEM/F-12 medium supplemented with 10% fetal bovine serum (FBS) (HyClone). Murine melanoma cell line B16-BL6 obtained from ATCC and B16-GM described previously (De Henau et al., 2016) were cultured in endotoxin-free RPMI1640 medium supplemented with 10% FBS (HyClone). All the cells are not among commonly misidentified cell lines, and were tested for myco-plasma contamination annually using a Mycoplasma Detection Kit (Biotool #B3903). In order to prevent potential contamination, all the media were supplemented with penicillin-streptomycin (15-140-122, Thermo Fisher Scientific) according to the manufac-turer's instructions.

Human samples. The untreated breast cancer samples including tumor tissues and blood plasma from TNBC patients who had undergone a mastectomy before therapy, normal breast tissues from women undergoing cosmetic breast surgery, and blood plasma from colon cancer patients and healthy subjects were collected at the First Affiliated Hospital of Chongqing Medical University. The blood plasma from patients with advanced lung (n=48), colon (n=12) and breast (n=10) cancers were collected pre-anti-PD1 treatment (patients were treated with Camrelizumab between Dec. 24, 2019 and Feb. 27, 2021) at the Beijing Friendship Hospital of Capital Medical. Best percentage change in the sum of the diameters for the selected target lesion is defined by Response Evaluation Criteria in Solid Tumors (RECIST version 1.1) on minimum 2 computed tomographic scans before treatment and 1 computed tomographic scan during treatment. The use of pathological specimens, as well as the review of all pertinent patient records, were approved by the Research Ethics Committee of the First Affiliated Hospital of Chongqing Medical University (project approval number 1005367 2017-012) and the Research Ethics Committee of Beijing Friendship Hospital of Capital Medical University (project approval number 2017-P2-141-01), and written informed consent were obtained from each participant and/or their legal representative, as appropriate. The information about the samples used is summarized in Table S7.

### Method Details

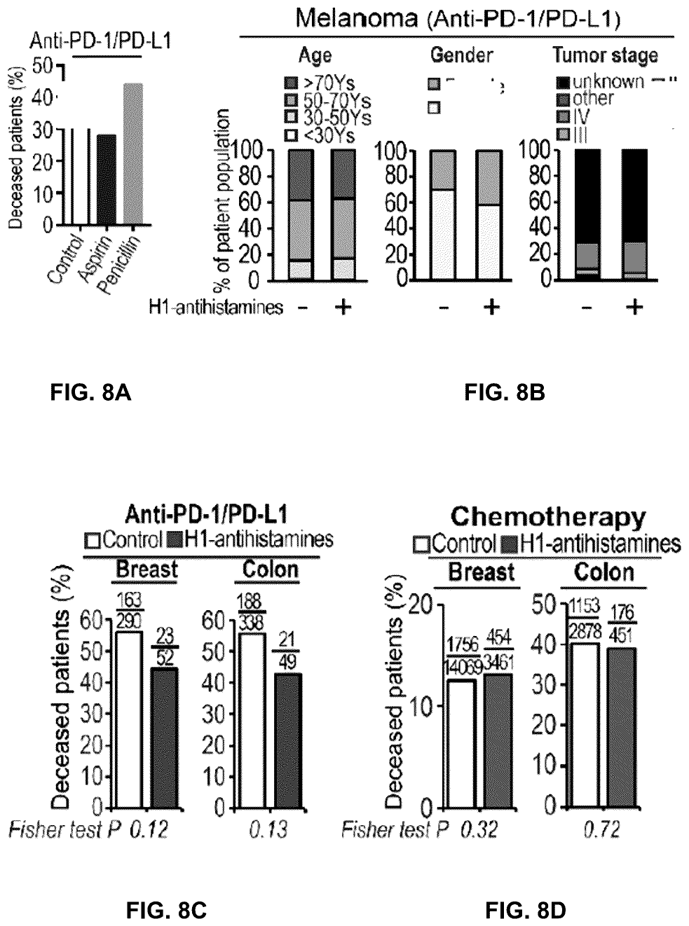

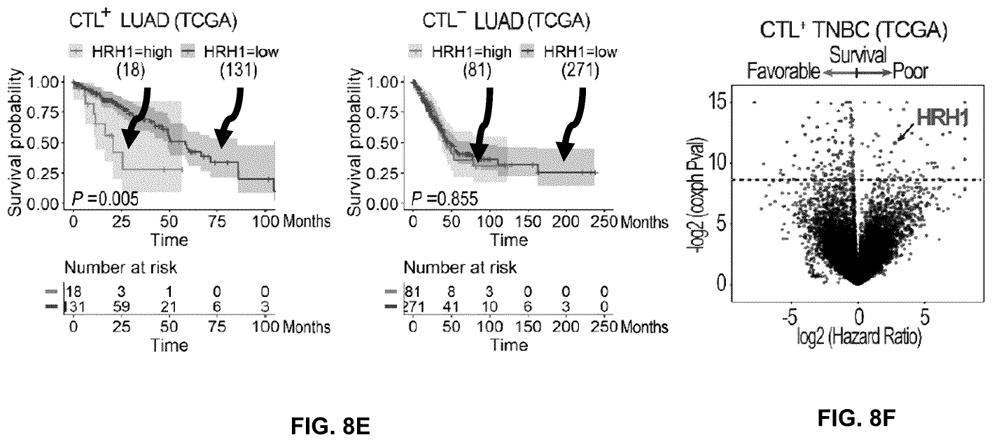

Survival analysis using The Cancer Genome Atlas (TCGA) data. TCGA breast cancer gene expression data and clinical data were downloaded from TCGA data portal (tcga-data.nci.nih.gov/tcga/dataAccessMatrix.htm). Triple-negative breast cancer (TNBC) cases were collected based on ESR1, PGR, and ERBB2 expres¬sion (RNAseq V2 RSEM) by using K-means 2 separation to define positive and negative groups and then combining triple-negative samples. CD8+ cytotoxic T lymphocyte (CTL) groups in TNBC samples were based on K-means 3 separation using a 15-gene signature (CD3E, CD8A, CCL2, CCL3, CCL4, CXCL9, CXCL10, GZMA, GZMK, HLA-DOA, HLA-DOB, HLA-DMB, ICOS, IRF1 and PRF1; FIG. 8A). Survival analysis was performed for 77 CTL-high TNBC cases with survival data using the coxph function of R software using a resampling procedure (Yao et al., 2012). Candidate prognostic genes were collected from 100-round runs with a coxph p value of <0.02 in >75 rounds. Volcano plots were drawn using hazard ratios and p values from the coxph analysis on 16,975 genes after removal of low expressers with candidate genes associated with poor prognosis and favorable prognosis marked in both CTL-high and CTL-low groups. To test the association between HRH1 gene expression level and patient survival, Kaplan-Meier survival analysis was performed using the similar program described in the Human Protein Atlas (www.proteinatlas.org/humanpathology) (Uhlen et al., 2017). Briefly, survival analysis was performed using R package survival and survmine. Patients were stratified into gene expression high and low groups using Kmeans two separation of gene expression values (consensus from 20 iterations), which is an unbiased separation based on gene expression dispersion.

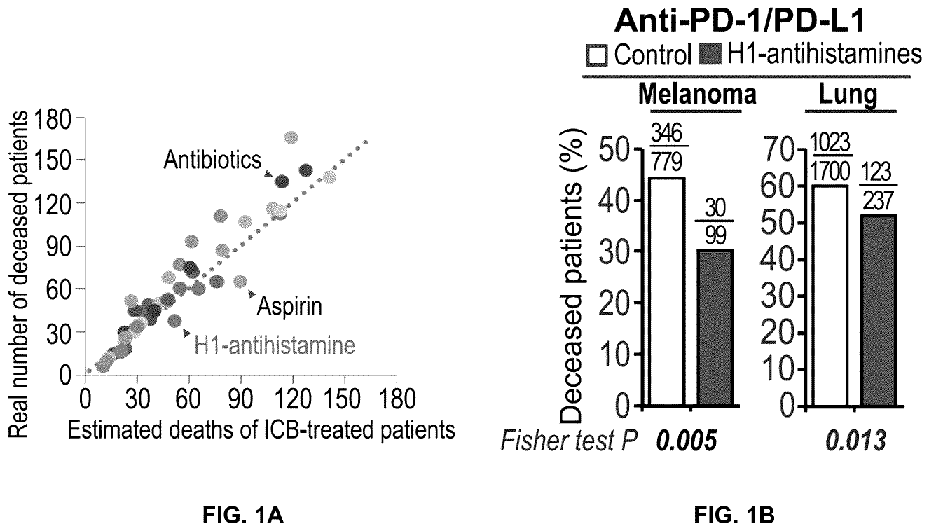

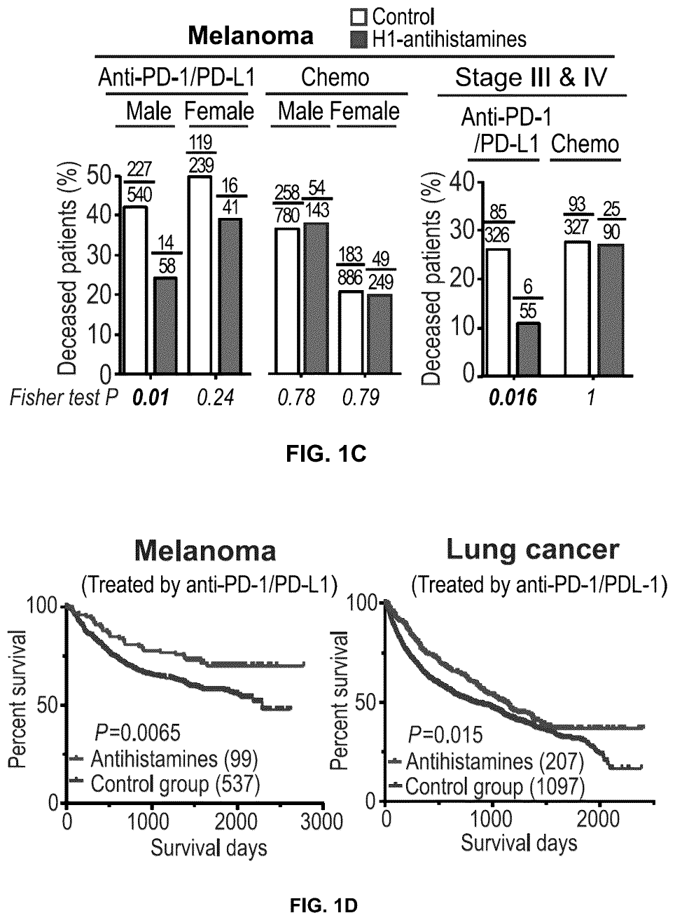

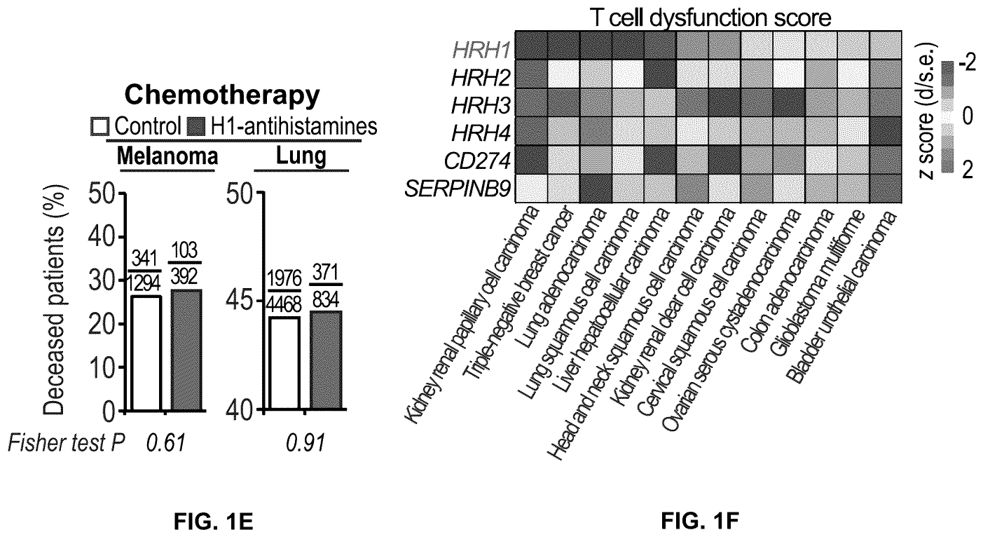

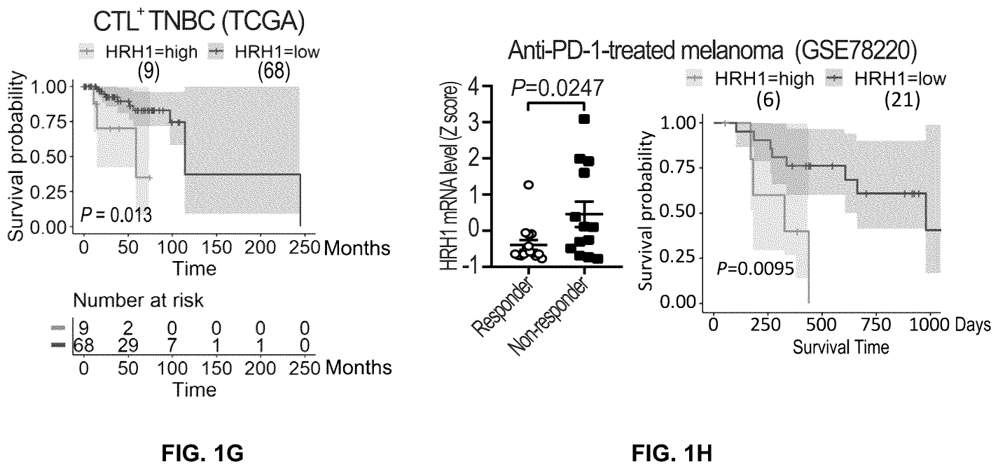

Epic SlicerDicer software analysis. Our retrospective study was conducted on melanoma patients encountered during 2016-2017 at The University of Texas MD Anderson Cancer Center, which draws a diverse range of local, regional, national, and international patients. The study population consisted of all melanoma patients with of International Classification of Diseases (ICD) code C43 (10th version). Anonymized aggregate-level data were collected using the SlicerDicer function within MD Anderson Epic electronic medical records. Institutional Review Board approval was therefore waived. Using the Epic SlicerDicer, we identified 9922 patients with a visit diagnosis, billing diagnosis, or active problem list with malignant skin of melanoma (ICD-10-CM:C43, which includes all subgroups). EPIC SlicerDicer was used to further identify 878 of these patients who received anti-PD-1 antibodies (pembrolizumab, nivolumab, or cemiplimab) or anti-PD-L1 antibodies (durvalumab, atezolizumab, or avelumab) treatment during the study period. The comparison group was 1986 patients with the same diagnosis who received chemotherapies but no immunotherapeutic treatment. To analyze the impacts of 40 different common pharmaceutical drugs (Table S1) on patients' outcomes, the 878 patients receiving anti-PD-1/PD-L1 treatment were further divided into 40 patient subgroups based on the medications they took during immunotherapeutic treatment. The estimated patient deaths of each subgroup were calculated based on the patient number of each subgroup and the average patient death rate (39%, based on deceased patients out of total patients analyzed). The real patient deaths of each subgroup were compared to the estimated patient deaths in order to determine the overall impact of the medication on clinical outcome (FIG. 1A). For further analysis of the H1-antihistamines, the 878 patients (received immunotherapy treatment) and 1986 patient (receiving chemotherapy treatment) were further subdivided into patients subgroups with or without uptake of H1-antihistamines that selectively target HRH1 (including fex-ofenadine, loratadine, desloratadine, cetirizine, levocetirizine, and azelastine) at the same time with anti-PD-1/PD-L1 antibody treatment or chemotherapies. Patient information, including age (<30 years, 30 to <50 years, 50-70 years, >70 years), sex (male or female), disease stage (0, I, II, III, IV, other, or unknown), survival (alive or dead), was extracted. Because many patients' direct responses to therapy were not available in Epic SlicerDicer, the overall survival (up to Jun. 15, 2021) was used as a surrogate indicator of patients' therapeutic response. The Fisher exact test was used to identify any nonrandom association between antihistamine uptake and patients' overall survival status. A similar analysis was performed in breast cancer patients (ICD code: C50) encountered at MD Anderson during 2016-2018, lung cancer patients (ICD code: C34) encountered at MD Anderson during 2016-2018, and colon cancer patients (ICD code: C18) encountered at MD Anderson during 2016-2018. To perform patient overall survival analysis, patient death date and the date receiving anti-PD-1 or PD-L1 treatment were pulled out from Epic. The survival time was calculated as the days between the two dates. To investigate the potential impact of melanoma patients' allergy status on their response to ICB therapies, melanoma patients who received anti-PD-1/PD-L1 immunotherapy were divided into two groups based on their allergy status. Patients were considered to have had an allergic response if they had a specified diagnosis of allergy status to other drugs, medi-caments, or biological substances (ICD code: Z88) documented as a visit diagnosis, billing diagnosis, or active problem list. To avoid conflating these allergic responses induced by anti-PD-1/PD-L1 antibodies, which may interfere with the treatment efficacy, we reserved the allergic response category for patients who reported an allergic response within 10 days before receiving anti-PD-1/PD-L1 treatment.

TABLE S1
Key resources used.

| | | |
|---|---|---|
| REAGENT or RESOURCE | SOURCE | IDENTIFIER |

Antibodies

| | | |
|---|---|---|
| Anti-mouse CD45 | BioLegend | Cat# 103110; RRID: AB_312975 |
| PE/Cyanine5 | | |
| Anti-mouse CD3e APC | BioLegend | Cat# 100312; RRID: AB_312677 |
| Anti-mouse CD8a | BioLegend | Cat# 100722; RRID: AB_312761 |
| PE/Cyanine7 | | |
| Anti-mouse/human | BioLegend | Cat# 101263; RRID: AB_2629529 |
| CD11b BV 510 | | |
| Anti-mouse Ly-6G/Ly- | BioLegend | Cat# 108408; RRID: AB_313373 |
| 6C (Gr-1) PE | | |
| Anti-mouse F4/80 FITC | BioLegend | Cat# 123108; RRID: AB_893502 |
| Anti-mouse CD206 PE | BioLegend | Cat# 141706; RRID: AB_10895754 |
| Anti-mouse I-A/I-E | BioLegend | Cat# 107616; RRID: AB_493523 |
| Alexa Fluor ® 488 | | |
| Anti-mouse VISTA | BioLegend | Cat# 143714; RRID: AB_2632662 |
| PE/Cyanine7 | | |
| Anti-mouse CD366 | BioLegend | Cat# 134004; RRID: AB_1626177 |
| (Tim-3) PE | | |
| Anti-mouse CD11c APC | BioLegend | Cat# 117310; RRID: AB_313779 |
| Anti-mouse CD24 Alexa | BioLegend | Cat# 101818; RRID: AB_493484 |
| Fluor ® 647 | | |
| Anti-mouse GITR | BioLegend | Cat# 120306; RRID: AB_2207248 |
| Ligand PE | | |
| Anti-mouse CD275 | BioLegend | Cat# 107405; RRID: AB_2248797 |
| (ICOS Ligand) PE | | |
| Anti-mouse 4-1BB | BioLegend | Cat# 107105; RRID: AB_2256408 |
| Ligand PE | | |
| Anti-mouse CD276 (B7- | BioLegend | Cat# 124508; RRID: AB_1279206 |
| H3) PE | | |
| Anti-mouse CD252 | BioLegend | Cat# 108810; RRID: AB_2207379 |
| Alexa Fluor ® 647 | | |
| Anti-mouse Galectin-9 | BioLegend | Cat# 136110; RRID: AB_2561658 |
| APC | | |
| Anti-mouse Ki-67 FITC | BioLegend | Cat# 652410; RRID: AB_2562141 |
| Anti-mouse PRF1 PE | BioLegend | Cat# 154306; RRID: AB_2721639 |
| Anti-mouse IFN-g PE | BioLegend | Cat# 505810; RRID: AB_315404 |
| Anti-mouse CD274 | BioLegend | Cat# 124314; RRID: AB_10643573 |
| PE/Cyanine7 | | |
| Anti-mouse CD3ε | BioLegend | Cat# 100372; RRID: AB_2800556 |
| antibody | | |
| Anti-mouse CD28 | BioLegend | Cat# 102132; RRID: AB_2810333 |
| antibody | | |
| Anti-mouse CD16/32 | BioLegend | Cat# 101302; RRID: AB_312801 |
| antibody | | |
| Anti-human HRH1 | R&D Systems | Cat# FAB4726R |
| Alexa Fluor ® 647 | | |
| Anti-mouse Siglec-F | BD Biosciences | Cat# 565526; RRID: AB_2739281 |
| PerCP-Cy ™ 5.5 | | |
| HRH1 antibody | Alomone Labs | Cat# AHR-001; RRID: AB_2039915 |
| HRH1 antibody | LifeSpan | Cat# LS-C331459 |
| | BioSciences | |
| HRH1 antibody | Abcam | Cat# ab75236; RRID: AB_2092479 |
| CD68 antibody | Abcam | Cat# ab955; RRID: AB_307338 |
| VISTA antibody | Cell Signaling | Cat# 54979; RRID: AB_2799474 |
| | Technology | |
| CD11b/c antibody | Novus Biologicals | Cat# NB110-40766; RRID: AB_714950 |
| β-actin | Santa Cruz | Cat # SC47778; RRID: AB_2714189 |
| | Biotechnology | |
| Anti-mouse IgG, | Thermo Fisher | Cat # 35510; RRID: AB_1185569 |
| DyLight 594 | Scientific | |
| Anti-Rabbit IgG, | Thermo Fisher | Cat # 35552; RRID: AB_844398 |
| DyLight 488 | Scientific | |
| Anti-mouse CTLA-4 | Bio X Cell | Cat # BE0164; RRID: AB_10949609 |
| (CD152) | | |
| Anti-mouse VISTA | Bio X Cell | Cat # BE0310; RRID: AB_2736990 |
| Anti-mouse CD8α | Bio X Cell | Cat # BE0117; RRID: AB_10950145 |
| CD45, Label: 89Y | Fluidigm | Cat # 3089005B; RRID: AB_2651152 |
| FoxP3, Label: 165Ho | Fluidigm | Cat # 3165024A; RRID: AB_2687843 |
| Granzyme B, Label: | Fluidigm | Cat # 3173006B; RRID: AB_2811095 |
| 173Yb | | |
| TIM-3, Label: 162Dy | Fluidigm | Cat # 3162029B; RRID: AB_2687841 |
| CD357, Label: 143Nd | Fluidigm | Cat # 3143019B |
| CD80, Label: 171Yb | Fluidigm | Cat # 3171008B |
| CD86, Label: 172Yb | Fluidigm | Cat # 3172016B |
| CD40, Label: 161Dy | Fluidigm | Cat # 3161020B |
| CD278, Label: 176Yb | Fluidigm | Cat # 3176014B |
| CD39, Label: 142Nd | Fluidigm | Cat # 3142005B |
| CD11b, Label: 148Nd | Fluidigm | Cat # 3148003B; RRID: AB_2814738 |
| CD11c, Label: 209Bi | Fluidigm | Cat # 3209005B; RRID: AB_2811244 |
| Ly-6C, Label: 150Nd | Fluidigm | Cat # 3150010B |
| Ly-6G, Label: 141Pr | Fluidigm | Cat # 3141008B; RRID: AB_2814678 |
| CD38, Label: 175Lu | Fluidigm | Cat # 3175014B |
| I-A/I-E, Label: 174Yb | Fluidigm | Cat # 3174003B |
| CD206, Label: 169Tm | Fluidigm | Cat # 3169021B; RRID: AB_2832249 |
| CD274, Label: 153Eu | Fluidigm | Cat # 3153016B; RRID: AB_2687837 |
| CD4, Label: 115In | BioLegend | Cat # 100506; RRID: AB_312709 |
| CD3e, Label: 152Sm | BioLegend | Cat # 100302; RRID: AB_312667 |
| CD8a, Label: 146Nd | BioLegend | Cat # 100702; RRID: AB_312741 |
| NK1.1, Label: 170Er | BioLegend | Cat # 108702; RRID: AB_313389 |
| CD19, Label: 149Sm | BioLegend | Cat # 115502; RRID: AB_313637 |
| T-bet, Label: 154Sm | BioLegend | Cat # 644825; RRID: AB_2563788 |
| IRF4, Label: 151Eu | BioLegend | Cat # 646402; RRID: AB_2280462 |
| CD152, Label: 163Dyv | BioLegend | Cat # 106202; RRID: AB_313247 |
| CD69, Label: 156Gd | BioLegend | Cat # 104533; RRID: AB_2563760 |
| CD14, Label: 158Gd | BioLegend | Cat # 123302; RRID: AB_940592 |
| F4/80, Label: 159Tb | BioLegend | Cat # 123102; RRID: AB_893506 |
| Ly-6G/C, Label: 139La | BioLegend | Cat # 108402; RRID: AB_313367 |
| CD103, Label: 147Sm | BioLegend | Cat # 121401; RRID: AB_535944 |
| GATA3, Label: 145Nd | Thermo Fisher | Cat # 14-9966-82; RRID: AB_1210519 |
| | Scientific | |
| Ki67, Label: 168Er | BD Biosciences | Cat # 556003; RRID: AB_396287 |
| CCR7, Label: 155Gd | Thermo Fisher | Cat # 16-1971-85; RRID: AB_494123 |
| | Scientific | |

Bacterial and virus strains

| | | |
|---|---|---|
| DH5α | Thermo Scientific | Cat# 18265017 |
| Stbl3 | Thermo Scientific | Cat# C737303 |

Biological samples

| | | |
|---|---|---|
| Human samples | The First | Project approval number 1005367 2017-012 |
| (Normal breast tissues, | Affiliated | |
| breast and colon cancer | Hospital of | |
| tissues) | Chongqing | |
| | Medical | |
| | University, China. | |
| The blood plasma from | Beijing Friendship | Project approval number 2017-P2-141-01 |
| patients with advanced | Hospital of | |
| lung (n = 48), colon (n = | Capital Medical | |
| 12) and breast (n = 10) | | |
| cancers collected pre- | | |
| anti-PD1 treatment | | |
| (patients were treated | | |
| with Camrelizumab | | |
| between Dec. 24, 2019, | | |
| and Feb. 27, 2021) | | |

Chemicals, peptides, and recombinant proteins

| | | |
|---|---|---|
| Fexofenadine HCl | Sigma-Aldrich | Cat # PHR1685; CAS: 153439-40-8 |
| Ovalbumin | Sigma-Aldrich | Cat # A5503; CAS: 9006-59-1 |
| Puromycin | Sigma-Aldrich | Cat# P8833; CAS: 58-58-2 |
| dihydrochloride | | |
| Collagenase A | Roche | Cat # 11088793001 |
| 16% Formaldehyde, | Pierce | Cat # 28906 |
| Methanol-free | | |
| Ionomycin, Calcium | Cell Signaling | Cat # 9995; CAS: 56092-82-1 |
| Salt | Technology | |
| BAPTA-AM | Selleckchem | Cat # S7534; CAS: 126150-97-8 |
| Recombinant Murine | PeproTech | Cat # 315-05 |
| IFN-γ | | |
| Recombinant Murine | PeproTech | Cat # 214-14 |
| IL-4 | | |
| Fixable Viability Dye | eBioscience | Cat # 65-0863-14 |
| eFluor ™ 450 | | |
| CD11b MicroBeads | Miltenyi Biotec | Cat # 130-049-601 |
| Cell-ID ™ Cisplatin | Fluidigm | Cat # 201064 |

Critical commercial assays

| | | |
|---|---|---|
| Histamine ELISA kits | Enzo Life | Cat # ENZ-KIT140-0001 |
| | Sciences | |
| iScript ™ cDNA | Bio-rad | Cat # 1708891 |
| Synthesis Kit | | |
| Intracellular Fixation & | eBioscience | Cat # 88-8824-00 |
| Permeabilization Buffer | | |
| Set | | |
| Maxima SYBR Green | Thermo Fisher | Cat# K0253 |
| qPCR Master Mix | Scientific | |
| QIAGEN Plasmid Maxi | QIAGEN | Cat# 12163 |
| Kit | | |
| ImmunoSpot ® Kits | Cellular | Mouse IFN-g Single-Color ELISPOT |
| | Technology | |
| | Limited | |

Deposited data

| | | |
|---|---|---|
| RNA-seq with mouse | This paper | GEO: GSE161484 |
| BMDM (Raw and | | |
| analyzed data) | | |
| RNA-seq with human | Hugo et al., 2016 | GEO: GSE78220 |
| melanomas | | |
| Single-cell RNA-seq of | Jerby-Arnon et al., | GEO: GSE115978 |
| human melanoma | 2018 | |
| Single-cell RNA-seq | This paper | SRA Run Selector Accession: PRJNA756466 |
| with CD45⁺ immune | | |
| cells isolated from | | |
| EO771 tumors | | |

Experimental models: Cell lines

| | | |
|---|---|---|
| 293T | ATCC | Cat# ACS-4500; RRID: CVCL_4V93 |
| 4T1 | ATCC | Cat# CRL-2539; RRID: CVCL_0125 |
| B16/BL6 | ATCC | Cat# CRL-6475; RRID: CVCL_0159 |
| BT20 | ATCC | Cat# HTB-19; RRID: CVCL_0178 |
| BT549 | ATCC | Cat# HTB-122; RRID: CVCL_1092 |
| CT26 | ATCC | Cat# CRL-2638; RRID: CVCL_7256 |
| EMT6 | ATCC | Cat# CRL-2755; RRID: CVCL_1923 |
| EO771 | ATCC | Cat# CRL-3461; RRID: CVCL_GR23 |
| HCC1806 | ATCC | Cat# CRL-2335; RRID: CVCL_1258 |
| HS578T | ATCC | Cat# HTB-126; RRID: CVCL_0332 |
| L929 | ATCC | Cat# CCL-1; RRID: CVCL_0462 |
| LLC1 | ATCC | Cat# CRL-1642; RRID: CVCL_4358 |
| MDA-MB-231 | ATCC | Cat# HTB-26; RRID: CVCL_0062 |
| MDA-MB-435 | ATCC | Cat# HTB-129; RRID: CVCL_0622 |
| MDA-MB-436 | ATCC | Cat# HTB-130; RRID: CVCL_0623 |
| THP-1 | ATCC | Cat# TIB-202; RRID: CVCL_0006 |
| B16-GMCSF | N/A | |

Experimental models: Organisms/strains

| | | |
|---|---|---|
| Mouse: C57BL/6 | The Jackson | Stock No: 000664 |
| | Laboratory | |
| Mouse: BALB/c | The Jackson | Stock No: 000651 |
| | Laboratory | |
| Mouse: B6.129P2- | The Jackson | Stock No: 029346 |
| Hrh1^{tm1Wtn}/BrenJ | Laboratory | |
| Mouse: PD-L1^{-/-} | Dr. Don L. | Chen et al. (2014) |
| | Gibbons | |

Oligonucleotides

| | | |
|---|---|---|
| See Table S2 for a | This paper | N/A |
| detailed primer list | | |
| Recombinant DNA | | |
| pLKO.1-shTGFB1-1 | Sigma-Aldrich | TRCN0000065993 |
| pLKO.1-shTGFB1-2 | Sigma-Aldrich | TRCN0000065997 |

Software and algorithms

| | | |
|---|---|---|
| FlowJo | BD Biosciences | www.flowjo.com/solutions/flowjo/downloads |
| GraphPad Prism | GraphPad | www.graphpad.com/scientific-software/prism/ |
| ImageJ | NIH | imagej.nih.gov/ij/ |
| R Studio | N/A | www.rstudio.com |
| Cytofkit | Jinmiao Chen Lab | github.com/JinmiaoChenLab/cytofkit2 |

RNA sequencing and data analysis. WT and HRH1−/− BMDM were cultured with TCM or DMEM medium for two days and then total RNA was purified using Trizol (Invi-trogen). After removing DNA by DNase, RNA was further purified using RNeasy MinElute Cleanup Kit (Qiagen). RNA samples were sent to UT health sequencing core for library construction and sequencing. After Raw fastq files from RNA sequencing were mapped to mm10 genome using STAR2, read counts for genes were prepared by htseq from which TPM were calculated. Principle component (PCA) analysis was done using R software. GSEA analysis was done using Java Web Start downloaded from www.gsea-msigdb.org/gsea/downloads.jsp. Differential expressed genes between the wild-type and HRH1-KO samples were obtained by using R package DESeq2 with filtering parameters of fold change above 3, adjusted p<0.01, and average log 2(TPM) in the high expression group above 0. Volcano plots were drawn using log 2 (fold change) and −log 10 (adjusted p), ceiled at 5 and 100 respectively. We obtained a list of M1 and M2 macrophage signatures genes from previous publication (Gerrick et al., 2018) and generated two customized terms (M1_UP_SHORT and M2_UP_SHORT) using only genes with fold changes above 5. These terms were appended to the gmt file in mySigDB and used for GSEA analysis. For graphing top GSEA terms, we calculated a p value score using the average of −log 10 (NOM p-val) and −log 10 (FDR q-val) listed in the GSEA report where p values less than 1e-9 were set to 1e-9.

Single-cell transcriptomic profiling. 0.2 3 106 EO771 tumor cells were transplanted into mammary fat pad of wide type or HRH1/mice. Three weeks later, tumors were harvested and 0.1 gram of tumor tissue of each tumor were collected. Three tumor tissues from the same group were randomly combined into one mixed sample and proceeded to digestion using 2 mg/ml collagenase A. Single-cell suspension was generated following the STAR methods described below in “Isolation of tumor-infiltrating cells”. 106 live CD45+ immune cells were sorted by flow cytometry and submitted to UTHealth Cancer Genomics Core (CGC) for sequencing. Cells were labeled with multiplexing oli-goes (#1000261, 10× Genomics, Pleasanton, CA) by following the cell multiplexing oligo labeling protocol (CG000391). The labeled samples were then pooled together. The single cell capture and library construction were performed by following the 10× Genomics Chromium Next GEM Single Cell 3′ Reagent Kits v3.1 protocol (CG000388). Briefly, the pooled samples were loaded onto Chromium Next GEM Chip G (PN-1000120, 10× Genomics, Pleasanton, CA) with partitioning oil and barcoded singe cell gel beads. The bar-coded and full-length cDNA is produced after incubation of the gel beads-in-emulsion (GEMs) and amplified via PCR for library construction. The library preparation was performed by following the protocol of Chromium Single Cell 3′ GEM, Library & Gel Bead Kit v3 (PN-1000121, 10× Genomics, Pleasanton, CA). The quality of the final libraries was examined using Agilent High Sensitive DNA Kit (#5067-4626) by Agilent Bioanalyzer 2100 (Agilent Technologies, Santa Clara, USA), and the library concentrations were determined by qPCR using Collibri Library Quantification kit (#A38524500, Thermo Fisher Scientific) on a QuantStudio3 (ThermoFisher Scientific). The libraries were pooled evenly and underwent for the paired-end sequencing on an Illumina NextSeq 550 System (Illumina, Inc.) using High Output Kit v2.5 (#20024907, Illumina, Inc.).

### Single-Cell Gene Expression Processing and Analysis

CellRanger (10×Genomics; v6.0.1) subcommand multi was used to process the raw sequencing reads and generate count matrix per sample (Zheng et al., 2017). Reads were aligned to the mouse genome assembly (version mm10) which is pre-built by 10× Genomics. Raw count matrices were then merged and analyzed using the Seurat R package (v4.0.1) (Hao et al., 2021). Cell matrices were initially filtered by removing cells with barcodes lower than 20 UMIs, with lower than 200 expressed genes, or with more than 10% of reads mapping to the mouse mitochondrial genes. To avoid low-quality cells, empty droplets or multiplets, we further filtered cells based on the number of unique genes detected in each cell, which is capped in the range from 2.5th to 97.5th percentile. Counts for the remaining cells were normalized against library size and regressed for the unwanted cycling bias among proliferating cells, using S and G2M phase scores calculated by the CellCycleScoring function in Seurat package. Scaled and centered read counts were used as gene expression for further analysis.

Uniform Manifold Approximation and Projection for Dimension Reduction (UMAP) was applied to visualize inferred cell clusters (cite: arXiv:1802.03426v3) based on the top 30 principal components. Automatic immune cell annotation was performed using scPred package (v 1.9.0) (Alquicira-Hernandez et al., 2019). The built-in annotated human PBMC datasets were combined and used as a reference to predict the major immune cell types in our in-house mouse single-cell dataset. Manual inspections were carried out to calibrate the automatic cell annotations by examining the most highly expressed marker genes between clusters, as well as literature- and database-derived cell markers. Murine M1- and M2-like cell markers were extracted from prior publication (Jablonski et al., 2015), while exhausted CD8+ T cell markers were downloaded from CellMarker database (Zhang et al., 2019). To examine the phenotype shift between mouse wildtype and knockout groups, we calculated the M1-like and M2-like macrophages and the exhausted CD8+ T cell expression-based scores at single-cell level. Specifically, we overlapped cell type-specific markers with the top 50 highly expressed genes between clusters, and then took the average expression value as the score for each set of cell type-specific markers. All the statistical analyses were conducted using R software (v4.0.4).

To examine correlation of HRH1 gene expression with M1/M2 macrophage markers (Martinez et al., 2006) in human cancer, we obtained scRNA-seq data from GSE115978 (Jerby-Arnon et al., 2018) which included macrophages and other immune cells isolated from melanoma patients. To deal with missing data points in scRNA-seq, we focused on macrophage cells with normalized TPM above 0.5 for HRH1 and other macrophage marker genes of interest. Pearson correlation and scatter plots were done using log 2 (1+TPM/10) values as describe in previous publication (Jerby-Arnon et al., 2018).

Generation of stable cells using lentiviral infection. Mouse TGF-b1-targeting shRNAs (shTGFB1-1: TRCN0000065993; shTGFB1-2: TRCN0000065994) were purchased from Sigma-Al¬drich. For lentiviral production, the lentiviral expression vector was co-transfected with the third-generation lentivirus packing vectors into 293T cells using LipoD293 DNA in vitro Transfection Reagent (SignaGen Laboratories). Then, 48-72 hours after transfection, can¬cer cell lines were stably infected with viral particles.

Generation of naïve bone marrow-derived macrophages (BMDMs). Bone marrow cells were collected from femurs obtained from 8- to 10-week-old C57BL/6 or BALB/c mice. After red blood cell lysis, bone marrow cells were seeded at a density of 5×106 cells/150×15 mm Petri dish and cultured at 37° C. in complete Dulbecco modified Eagle medium (DMEM) containing 20% L929 cell-conditioned medium, providing macrophage colony-stimulating factor. Macrophages were ready for use on day 7 following a fresh medium change on day 4.

Isolation of murine peritoneal macrophages. 2 ml of 3% Brewer thioglycollate medium per mouse was injected into the peritoneal cavity to trigger an inflammatory response. Allow inflammatory response to proceed for 4 days, and then collect peritoneal exudate macrophages. After red blood cell lysis, peritoneal macrophages were cultured at 37° C. in complete DMEM medium, and ready for use.

In vitro co-culture with T cells. Spleens from wild-type C57BL/6 or BALB/c mice were harvested and filtered through a 40-mm cell strainer to generate a single-cell suspension. After red blood cell lysis, splenocytes were counted and plated in complete Roswell Park Memorial Institute 1640 medium supplemented with 50 mM b-mercaptoethanol and 10 mM HEPES onto 12-well plates coated with 2.5 mg/ml anti-CD3 (clone 145-2C11, BioLegend) and 3 mg/ml anti-CD28 (clone 37N, BioLegend) antibodies. Spleen T cells were activated for 48 hours before co-culture with macrophages. To educate the macrophages, we co-cultured naïve BMDMs with tumor cells at a ratio of 1:1 or with tumor cell-derived conditioned medium for 48 hours. Macrophages were seeded with activated T cells at a ratio of 5:1. After co-culture for another 24 or 48 hours, T cells were collected for flow cytometry analysis.

In vitro T cell killing assays. Ovalbumin (OVA)-expressing EO771 cells alone or co-cultured with macrophages at a ratio of 1:2 were plated into 12-well plates. The following day, OT-I T cells pre-activated by OVA257-264 were added to the plates at the indicated ratio. After co-culture for another 48 hours, the tumor cells were harvested and counted to determine T cell killing ability.

Macrophage polarization and stimulation. To polarize macrophages toward an M1-like phenotype, we stimulated PBMC-derived macrophages or BMDMs with IFN-g (20 ng/ml, PeproTech) for 48 hours. To induce an M2-like phenotype, we treated THP-1-derived macrophages or BMDMs with IL-4 (20 ng/mL, PeproTech) for 48 hours. To stimulate macrophages with histamine, we cultured peritoneal macrophages with histamine (10 mM) for 48 hours. To stimulate macrophages with tumor cell-derived conditioned medium (TCM), we cultured BMDMs with complete medium containing 50% TCM (volume).

Tumor inductions and treatment experiments. For 4T1 and EO771 models, 2×10⁵ tumor cells were orthotopically injected into female BALB/c or C57BL/6 mice, respectively. For LLC and B16-GM models, 2×10⁵ tumor cells were subcutaneously injected into the back of C57BL/6 mice. For the CT26 model, 2×10⁵ CT26 cells were subcutaneously injected into the back of BALB/c mice. The induction of allergic airway disease has been described previously. Briefly, BALB/c mice were sensitized using OVA (Sigma-Aldrich) at a dose of 0.01 mg/mouse in 0.2 ml incom¬plete Freund's adjuvant intraperitoneally on days 0 and 12. Control mice received the same volume of phosphate-buffered saline in incomplete Freund's adjuvant. All groups of mice were challenged daily with 5% OVA (aerosolized for 20 minutes) via the airways between days 19 and 24. EMT6 and CT26 models were constructed by orthotopic injection of 2×105 tumor cells on day 18 after the first OVA sensitization. Treatments were given as single agents or in combination, with the following regimen for each drug. HRH1 antagonist fexofenadine hydrochloride was administered by oral gavage once per day at 30 mg/kg. Treatments were initiated on day 5 after tumor inoculation for the entire duration of the experiment. Anti-PD-1 antibody (clone RMP1-14; the hybridoma RMP1-14 for aPD-1 production was provided by Dr. Hideo Yagita, 10 mg/kg) and anti-CTLA-4 antibody (clone 9D9, Bio X Cell, 5 mg/kg) were injected intra-peritoneally on days 7, 10, 13, 16, and 19 after tumor inoculation. Anti-VISTA antibody (clone 13F3, Bio X Cell, 300 mg/mouse) was injected subcutaneously every day for 10 days starting on day 7, followed by continuous injection every 2 days for the rest of the duration of the experiment (Le Mercier et al., 2014). For in vivo CD8+ T cell depletion, mice were treated with 200 mg of anti-CD8 antibody every 3-4 days starting at 3 days before B16-GM tumor inoculation. For in vivo macrophage adoptive transfer experiments, B16-GM tumor cells mixed with primary wild-type or HRH1−/− BMDMs at a ratio of 1:5 were injected subcutaneously into HRH1−/− or wild-type host mice, respectively. For the re-challenge study, mice with complete responses were re-challenged with 2×105 B16-GM or EO771 tumor cells (on day 150 after tumor implant). Tumor size was measured by calipers every second or third day when tumors were palpable, and the volume was calculated using the formula V=as (width×width×length)/2.

Bone marrow transplantation. Bone marrow transplantation followed previous publication (Khononov et al., 2021). In brief, on day 1, recipient mice (8 weeks old) received 1,000 rads total body irradiation (¹³⁷Cesium Gammacell source), and 4 hours later, they were transplanted with 5 3 106 bone marrow cells collected from femurs of the donor mice (5 weeks old) via tail vein injection. In a successful graft, the immunological reconstitution is expected complete within 4-5 weeks. 30 days after bone marrow transplantation, the recipient mice were ready for following experiments.

Isolation of tumor-infiltrating cells. Mouse tumor samples were chopped with scissors and then subjected to enzymatic digestion with 2 mg/ml collagenase A (Roche) in DMEM for 1 hour at 37° C. Next, tissues were filtered through 70-mm filters (BD Biosciences) to achieve single-cell suspensions. After treatment with red blood cell lysis buffer for 5 minutes at room temperature, all samples were washed and re-suspended in flow cytometry buffer (phosphate-buffered saline/0.5% albumin/2 mM EDTA) or DMEM depending on further use.

Flow cytometry staining and analysis. Live single cells were sub-gated by staining with Fixable Viability Dye eFluor 450 (eBioscience) for 15 minutes at 4° C. For blocking of Fc receptors, cells were then pre-incubated with purified anti-CD16/32 antibody (clone 93, BioLegend) for 10 minutes on ice before immunostaining. After one wash with flow cytometry buffer, cells were incubated with appropriate dilutions of various combinations of the following antibodies. Primary antibodies to cell surface markers directed against CD45 (30-F11), CD3 (145-2C11), CD8a (53¬6.7), CD11b (M1/70), Gr-1 (RB6-8C5), F4/80 (BM8), CD206 (C068C2), I-A/I-E (M5/114.15.2), VISTA (MHSA), Tim-3 (B8.2C12), CD11c (N418), CD24 (M1/69), GITRL (YGL 386), ICOSL (HK5.3), 4-1BBL (TKS-1), CD276 (RTAA15), OX40L (RM134L), Galectin-9 (RG9-35), PD-L1 (10F.9G2), and PD-L2 (TY25) were from BioLegend; against HRH1 (480054), from R&D Systems; against HRH1 (AHR-001), from Alomone Labs; and against Siglec-F (E50-2440), from BD Biosciences. For intracellular staining, cells were fixed, permeabilized using Foxp3/Transcription Factor Staining Buffer Set (eBioscience), and then stained with fluorochrome-conjugated antibodies to Ki-67 (16A8) and PRF1 (S16009A) from BioLegend. For cytokine staining, cells were first stimulated with Cell Stimulation Cocktail (eBioscience) at 37° C. for 4 hours, and then stained with anti-IFN-g (XMG1.2) from BioLegend. The stained cells were acquired by a BD FACSCanto II Flow Cytometer using BD FACSDiva software (BD Biosciences), and data generated were processed using FlowJo software.

Mass cytometry and data analysis. Mouse tumor tissues were digested as described above. Then, for CyTOF analysis, cells were incubated with 25 mM cisplatin for 1 minute (viability staining) and subsequently stained with a metal-labeled monoclonal antibody cocktail against cell surface molecules. After treatment with the Fixation/Permeabilization Buffer (eBioscience), cells were further incubated with monoclonal antibody cocktails against intracellular proteins. Antibodies used in the mass cytometry analysis were purchased from Fluidigm. The samples were analyzed using the CyTOF 2 instrument (Fluidigm) in the Flow Cytometry and Cellular Imaging Core Facility at MD Anderson. All CyTOF files were normalized and manually gated in Cytobank software. Data were transformed using the cytofAsinh function before they were applied to the downstream analysis. Phenograph clustering analysis in the R cytofkit package was performed on pooled samples to automatically identify underlying immune subsets. Heat-maps were generated on the basis of the mean value for each marker in clusters. Cell frequency in each cluster was calculated as the assigned cell events divided by the total CD45+ cell events in the same sample.

Purification of myeloid cells or macrophages from tumors. Single-cell suspensions of mouse tumors were generated as described in the previous section. Single cells were stained with CD11b microbeads (Miltenyi Biotec) according to the manufacturer's instructions to enrich the myeloid fractions. Cells were then stained with Fixable Viability Dye eFluor 450 (eBioscience) to exclude dead cells, and anti-Gr-1-phycoerythrin (PE) (clone RB6-8C5) and anti-F4/80-fluorescein isothiocyanate (FITC) (clone BM8) for flow sorting on a FACSAria II Cell Sorter (BD Biosciences).

qRT-PCR. cDNA was prepared using 1 mg of RNA with the iScript cDNA Synthesis Kit (Bio-Rad). SYBR green-based qRT-PCR was performed using mouse primers to Il1b, Il6, Il10, Il12b, Nos2, Arg1, Tgfb1, Vista, Hrh1, and 18s (Integrated DNA Technologies). mRNA levels were normalized to 18s (DCt=Ctgene of interest-Ct18s) and presented as relative mRNA expression (DDCt=2−(ΔCtsample−DCtcontrol)) or fold change.

Western blotting. Western blotting was done as previously described (Zhang et al., 2015). The following primary antibodies were used: HRH1 (LS-C331459, LifeSpan BioSciences), VISTA (54979, Cell Signaling Technology) and CD11b (NB 110-40766, Novus Biologicals).

Enzyme-linked immunosorbent assay (ELISA). The levels of histamine in cell culture supernatant, serum/plasma, and tissues were detected by Histamine ELISA kits (ENZ-KIT140-0001, Enzo; ab213975, Abcam) according to the manufacturer's instructions.

IFN-γ ELISPOT assay. The IFN-7 ELISPOT assay was done following the manufacturer's protocol. Briefly, the CD45+ leukocytes were sorted from tumors by flow cytometry. The leukocytes were counted and seeded at 5×10⁵ cells/well into pre-coated PVDF plates (ImmunoSpot® Kits, Cellular Technology Limited), stimulated with anti-CD3 antibody and IL-2 overnight, and secreted IFN-g was quantified following standard protocol. Assay plates were scanned and analyzed using an automated ELISPOT reader system.

Immunohistochemistry (IHC) and immunofluorescence (IF) staining. Standard IHC and IF staining was performed as described previously (Zhang et al., 2020). The primary antibodies used for IHC staining include anti-GZMB (ab255598, Abcam), anti-HDC (ab37291, Abcam), and anti-CD31 (77699, Cell Signaling); used for IF staining include anti-CD68 (ab955, Abcam) and anti-HRH (ab75236, Abcam). DyLight 488- or DyLight 594-conjugated secondary antibodies against rabbit or mouse IgG were purchased from Thermo Fisher Scientific.

Intracellular calcium measurement. Intracellular Ca2+ was determined using the Fluo-Forte calcium assay kit (Enzo Life Sciences) according to the manufacturer's instructions. Fluorescence was measured using a BD FACSCanto II Flow Cytometer.

### Quantification and Statistical Analysis

Prism 8.0 software (GraphPad) was used for statistical analysis. Analysis for significance was performed by one-way or two-way ANOVA when more than two groups were compared and by parametric or nonparametric Student t-test when only two groups were compared. Fisher exact test was used when percentages of cancer patients from different groups were compared. Chi-square test was performed to determine whether there was any significant difference of gender, age, and tumor stages between the patient groups that took antihistamines and that didn't take antihistamines. p<0.05 was considered statistically significant (*p<0.05, **p<0.01, ***p<0.001, ****p<0.0001). Survival was evaluated using the Kaplan-Meier method and analyzed by the Mantel-Cox log rank test. All experiments were performed at least twice, and n refers to biological replicates.

### Example 2. The Allergy Mediator Histamine Confers Resistance to Immunotherapy in Cancer Patients Via Activation of the Macrophage Histamine Receptor H1

### Summary

Reinvigoration of antitumor immunity remains an unmet challenge. Our retrospective analyses revealed that cancer patients who took antihistamines during immunotherapy treatment had significantly improved survival. We uncovered that histamine and histamine receptor H1 (HRH1) are frequently increased in the tumor microenvironment and induce T cell dysfunction. Mechanistically, HRH1-activated macrophages polarize toward an M2-like immunosuppressive phenotype with increased expression of the immune checkpoint VISTA, rendering T cells dysfunctional. HRH1 knockout or antihistamine treatment reverted macrophage immunosuppression, revitalized T cell cytotoxic function, and restored immunotherapy response. Allergy, via the histamine-HRH1 axis, facilitated tumor growth and induced immunotherapy resistance in mice and humans. Importantly, cancer patients with low plasma histamine levels had a more than tripled objective response rate to anti-PD-1 treatment compared with patients with high plasma histamine. Altogether, pre-existing allergy or high histamine levels in cancer patients can dampen immunotherapy responses and warrant prospectively exploring antihistamines as adjuvant agents for combinatorial immunotherapy.

### Results

Patients Receiving Antihistamines have Better Survival with ICB Therapies

To assess the impact of taking other medications on the therapeutic response to immunotherapy in cancer patients, we retrospectively evaluated the clinical outcomes of melanoma patients who took another medicine among 40 charted common drugs (Table S1) while receiving immunotherapy (anti-PD-1/PD-L1) at The University of Texas MD Anderson Cancer Center (MDACC). Our data showed that taking antibiotics (e.g., ampicillin) was associated with an increased death rate in immunotherapy treated patients, consistent with a previous report (Elkrief et al., 2019), whereas taking aspirin was correlated with a reduced death rate in immunotherapy-treated patients, as found in mouse models (FIGS. 1A and 8A) (Zelenay et al., 2015). Among the 40 common drugs examined, only HRH1-specific antihistamines (H1-antihistamines or second-generation antihistamines) significantly correlated with better survival of patients, except for aspirin (FIG. 1A). Clearly, melanoma patients who took H1-antihistamines during anti-PD-1/PD-L1 treatments had a highly significantly reduced death rate compared with age-, sex-, or stage-matched patients who did not take the H1-antihistamines (FIGS. 1B, 1C, and 8B; Table S2). Among lung cancer patients receiving anti-PD-1/PD-L1 treatments, those taking H1-antihistamines also showed a statistically significant reduction in death rate compared with those who did not take the H1-antihistamines (FIG. 1B; Table S2). Kaplan-Meier survival analysis also indicated significantly improved overall survival in melanoma and lung cancer patients who took H1-antihistamines during anti-PD-1/PD-L1 treatment compared with control groups that did not (FIG. 1D). In addition, breast and colon cancer patients taking H1-antihistamines while receiving anti-PD-1/PD-L1 therapy also showed trends of reduced death rate (FIG. 8C; Table S2), although with no statistical significance, due to relatively smaller numbers of patients taking the H1-antihistamines. These clinical data indicated that H1-antihistamines may augment immunotherapy. Notably, H1-antihistamines had minimal effect on the survival of chemotherapy-treated patients (FIGS. 1E and 8D), suggesting that H1-antihistamines may not target tumor cells directly.

TABLE S2
Information of antihistamines uptake in
cancer patients, related to FIG. 1.

| Patient | | | Patient | Antihistamines uptake** | | |
|---|---|---|---|---|---|---|
| cancer type | Category | | No. | YES | NO | P-value |
| Melanoma | Gender | Male | 598 | 58 | 540 | 0.031 |
| patients | | Female | 280 | 41 | 239 | |
| (2016-2017) | | Sum | 878 | 99 | 779 | |
| | Age | <50 | 131 | 14 | 117 | 0.896 |
| | (years) | 50-70 | 380 | 45 | 335 | |
| | | >70 | 367 | 40 | 327 | |
| | | Sum | 878 | 99 | 779 | |
| | Stage* | 0-I | 23 | 2 | 21 | 0.153 |
| | | II | 15 | 0 | 15 | |
| | | III | 83 | 14 | 69 | |
| | | IV | 298 | 41 | 257 | |
| | | Others & | 502 | 51 | 451 | |
| Breast | Gender | Male | | NA | NA | |
| Cancer | | Female | 342 | 52 | 290 | |
| Patients | Age | <50 | 68 | 10 | 58 | 0.992 |
| (2016-2018) | (years) | 50-70 | 157 | 24 | 133 | |
| | | >70 | 117 | 18 | 99 | |
| | | Sum | 342 | 52 | 290 | |
| | Stage* | 0-I | 27 | 6 | 21 | 0.3315 |
| | | II | 44 | 6 | 38 | |
| | | III | 57 | 5 | 52 | |
| | | IV | 90 | 16 | 74 | |
| | | Others & | 173 | 28 | 145 | |
| Lung | Gender | Male | 1137 | 135 | 1002 | 0.562 |
| Cancer | | Female | 800 | 102 | 698 | |
| Patients | | Sum | 1937 | 237 | 1700 | |
| (2016-2018) | Age | <50 | 130 | 12 | 118 | 0.400 |
| | (Years) | 50-70 | 969 | 115 | 854 | |
| | | >70 | 838 | 110 | 728 | |
| | | Sum | 1937 | 237 | 1700 | |
| | Stage* | 0-I | 79 | 11 | 68 | 0.698 |
| | | II | 56 | 7 | 49 | |
| | | III | 113 | 12 | 101 | |
| | | IV | 466 | 69 | 397 | |
| | | Others & | 1284 | 149 | 1135 | |
| Colon | Gender | Male | 205 | 26 | 179 | 0.989 |
| Cancer | | Female | 182 | 23 | 159 | |
| Patients | | Sum | 387 | 49 | 338 | |
| (2016-2018) | Age | <50 | 79 | 10 | 69 | 0.857 |
| | (years) | 50-70 | 194 | 23 | 171 | |
| | | >70 | 114 | 16 | 98 | |
| | | Sum | 387 | 49 | 338 | |
| | Stage* | 0-I | 4 | 1 | 3 | 0.898 |
| | | II | 6 | 1 | 5 | |
| | | III | 17 | 2 | 15 | |
| | | IV | 120 | 22 | 98 | |
| | | Others & | 253 | 26 | 227 | |

HRH1 Correlates with T Cell Dysfunction in Human Cancers

The above clinical findings suggested that H1-antihistamines may enhance antitumor immunity. H1-antihistamines specifically block histamine binding to HRH1. Therefore, we examined whether HRH1 high-expressing tumors were associated with suppressed antitumor immunity in cancer patients. We adapted the Tumor Immune Dysfunction and Exclusion computational framework (Jiang et al., 2018) to evaluate the impacts of HRH1 on T cell infiltration and T cell dysfunction in patient samples from The Cancer Genome Atlas (TCGA). Evidently, HRH1 expression yielded high tumor immune dysfunction scores in 9 of 12 TCGA cancer types analyzed, a higher proportion than CD274 (PD-L1) and SERPINB9 (FIG. 1F), two genes well known for inducing T cell dysfunction (Jiang et al., 2018). Notably, HRH1 expression was not associated with cytotoxic T lymphocyte (CTL) infiltration (data not shown, see FIG. E FIGS. of U.S. Provisional Application No. 63/299,736), suggesting that HRH1 high expression may primarily induce T cell dysfunction. In contrast, other histamine receptors (HRH2/3/4) had much lower effect on T cell dysfunction compared with HRH1 (FIG. 1F). The strong association between HRH1 expression and T cell dysfunction prompted us to examine whether high HRH1 expression correlates with poor clinical outcome in cancer patients, especially in patients with CTL-infiltrated (CTL+) tumors (data not shown, see FIG. S1F of U.S. Provisional Application No. 63/299,736). Indeed, high HRH1 expression was significantly associated with poor survival in patients with CTL+triple-negative breast cancer (TNBC) and lung adenocarcinoma as well as a strong trend of poor survival in melanoma patients (FIGS. 1G and 8E; data not shown, see FIGS. S1G-S1I of U.S. Provisional Application No. 63/299,736). Notably, H1RH1 is among the top 20 genes that are strongly associated with poor survival in CTL+TNBC patients (hazard ratio>2; FIG. 8F).

Given that HRH1 is associated with T cell dysfunction in cancer patients and that patients receiving H1-antihistamines along with ICB had better survival, we further explored whether high HRH1 expression is associated with immunotherapy resistance. Among melanoma patients treated with the anti-PD-1 drug pembrolizumab (GSE78220) (Hugo et al., 2016), non-responders had higher HRH1 mRNA expression in pre-treatment tumors than did responders (FIG. 1H, left). Anti-PD-1-treated patients with high-HRH1-expressing tumors had devastatingly short overall survival compared with patients with low-HRH1-expressing tumors (FIG. 1H, right).

### Histamine and HRH1 are Upregulated in the Tumor Microenvironment

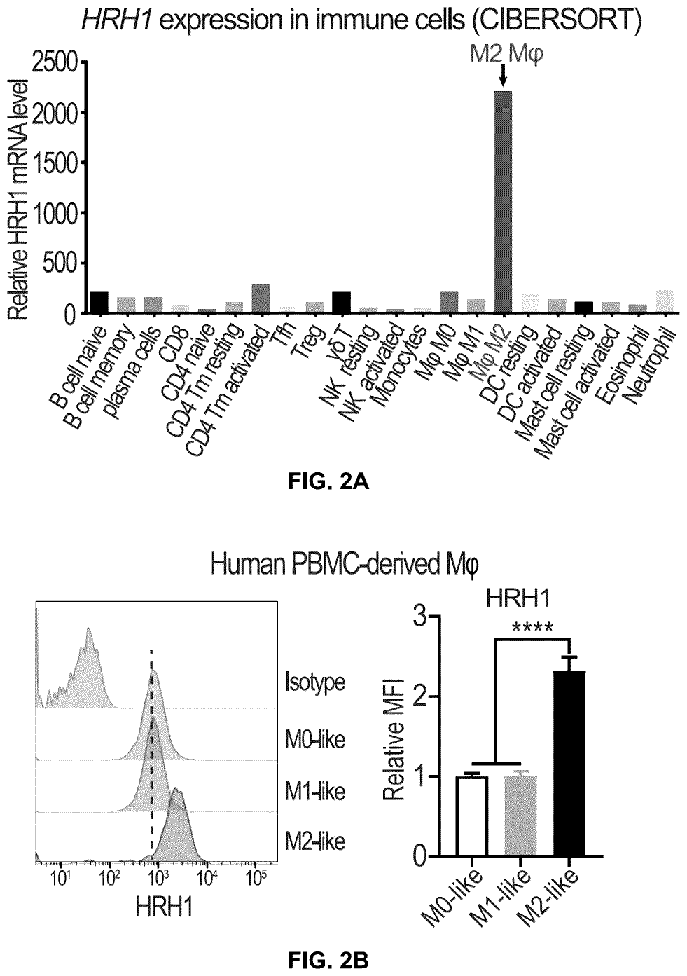

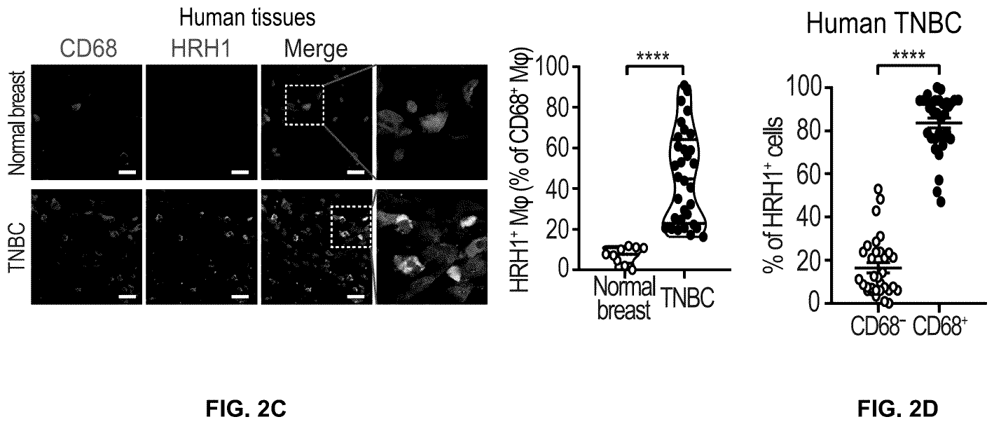

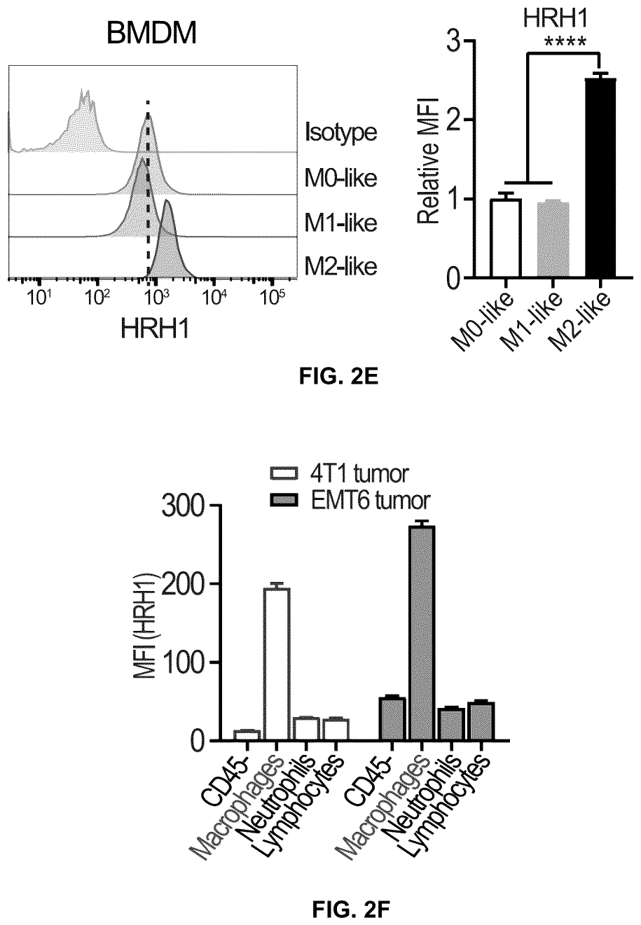

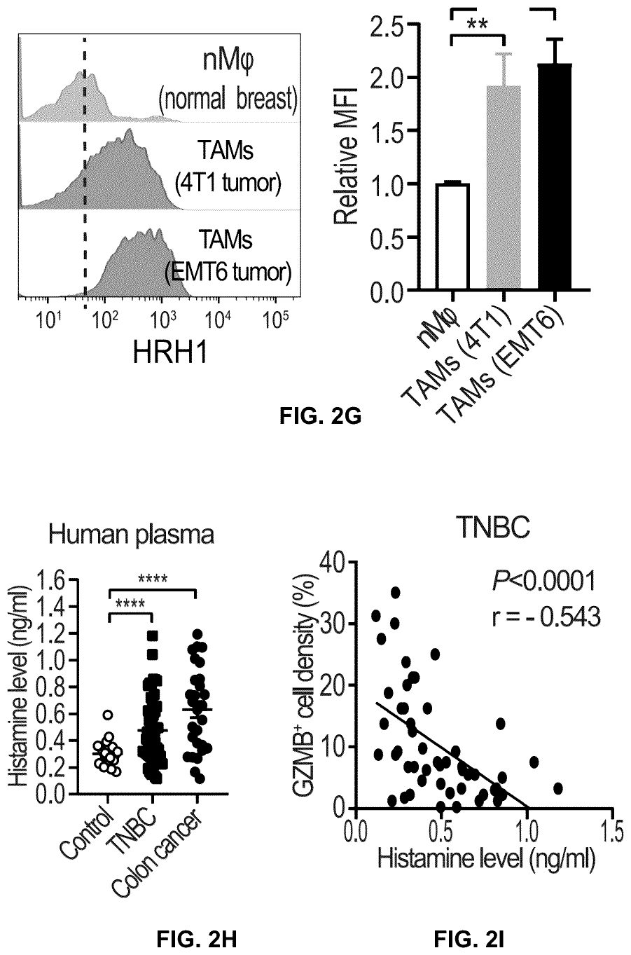

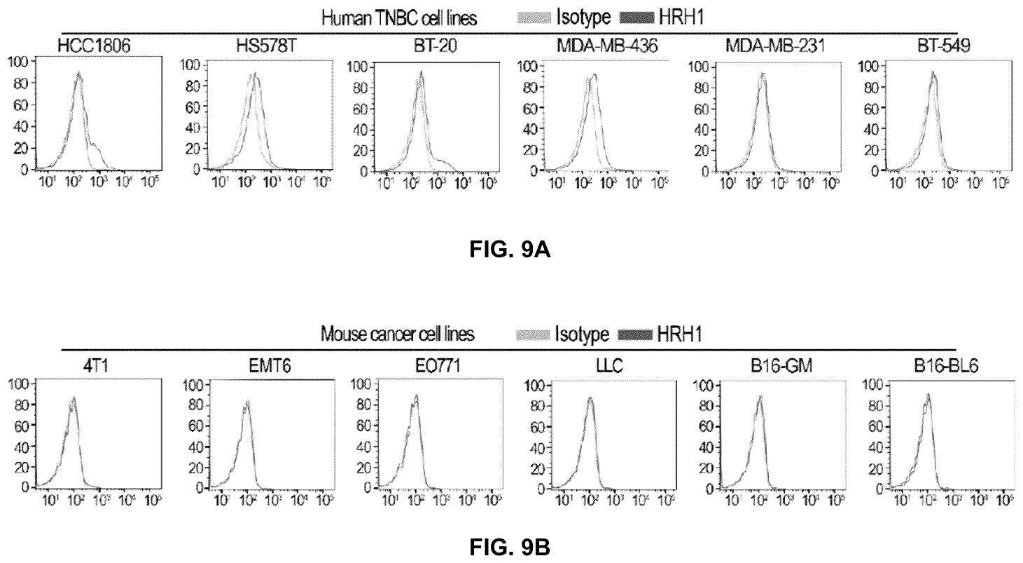

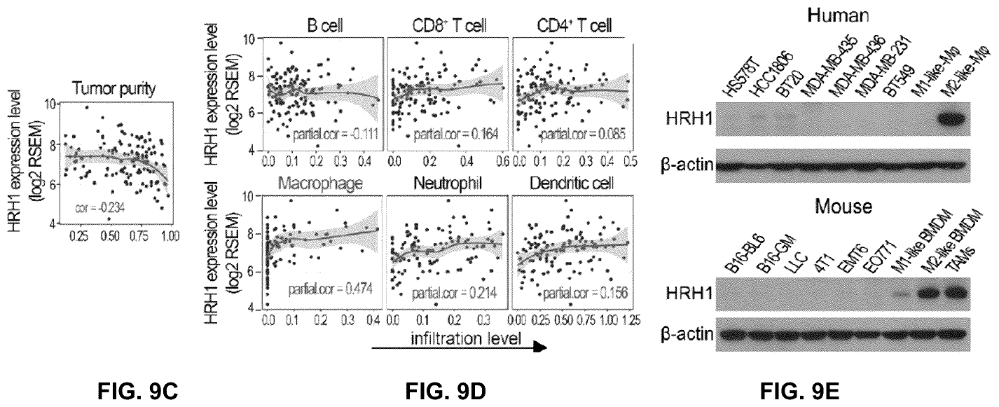

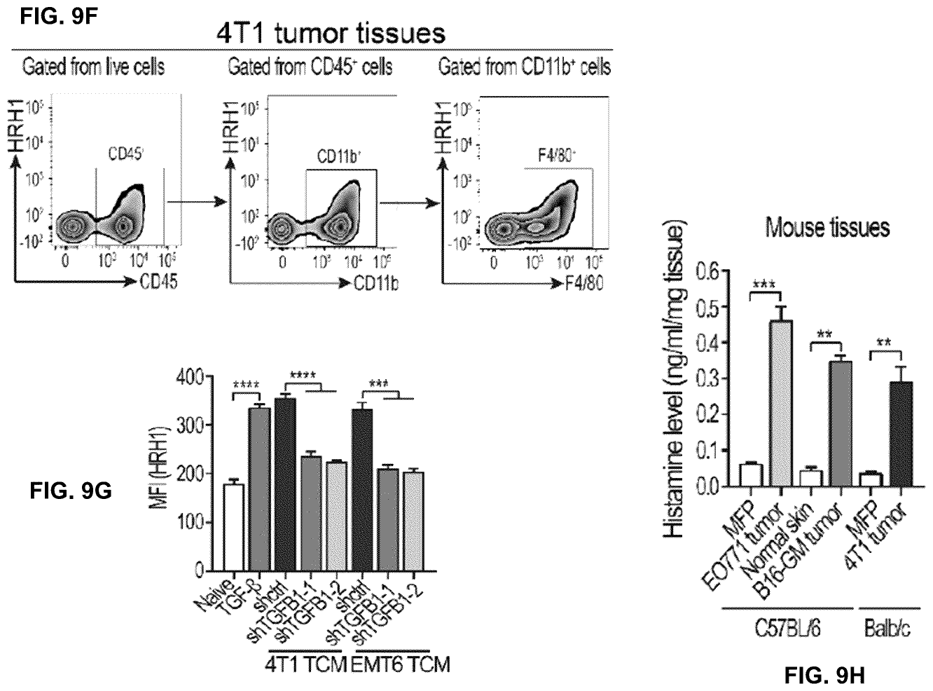

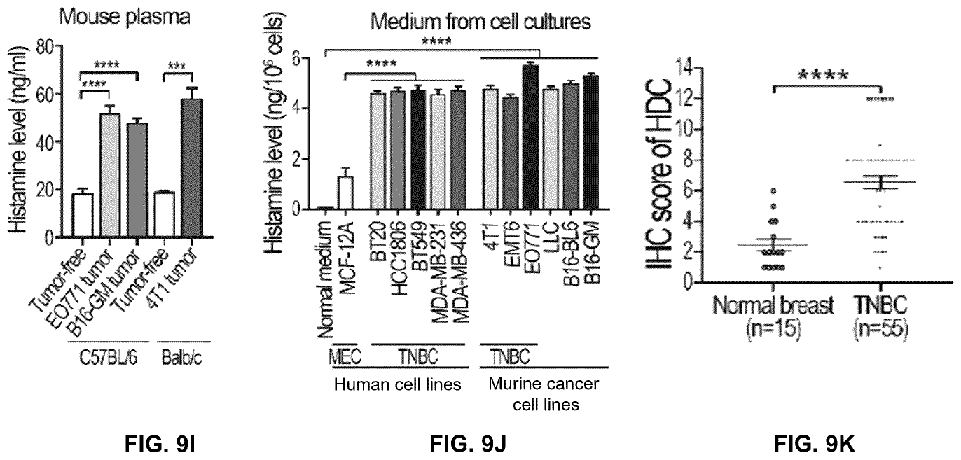

When deciphering how HRH1 induces T cell dysfunction, we surprisingly found that HRH1 expression was barely detectable in most of the tested human and mouse tumor cell lines (FIGS. 9A and 9B). Instead, using two deconvolution algorithms, i.e., Tumor Immune Estimation Resource (TIMER, Li et al., 2017) and CIBERSORT (Newman et al., 2015), we found that HRH1 expression was negatively correlated with tumor purity but positively correlated with tumor-associated macrophages (TAMs) in the tumor microenvironment (TME) (FIGS. 9C and 9D), particularly in immunosuppressive M2-like macrophages among various cell types in the human TME (FIG. 2A). Furthermore, HRH1 was expressed mainly on M2-polarized (interleukin [IL]-4-treated) macrophages and TAMs in the TME, instead of naive macrophages, M1-polarized (interferon g [IFN-g]-treated) macrophages, or resident macrophages from normal mammary glands, in both humans (FIGS. 2B-2D and 9E, top) and mice (FIGS. 2E-2G, 9E, bottom, and 9F). In addition to HRH1 upregulation on IL-4-induced M2-like macrophages (Orecchioni et al., 2019) (FIGS. 2B, 2E, and 9E), tumor-derived transforming growth factor b (TGF-b) also induced HRH1 expression on macrophages (FIG. 9G). In addition, we detected significantly increased levels of HRH1 ligand histamine in the blood of TNBC or colon cancer patients compared with those of healthy subjects (FIG. 2H). Intriguingly, high histamine levels in TNBC patients' blood were significantly correlated with low density of tumor-infiltrating granzyme B (GZMB)+ cells (cytotoxic CD8+ T cells or natural killer [NK] cells) (FIG. 2I), suggesting a potential link between histamine levels and immune cytotoxic cell infiltration. Increased histamine levels were also detected in tumor tissues (FIG. 9H) and blood of tumor-bearing mice (FIG. 9I), compared with corresponding normal tissues and blood from tumor-free mice, consistent with other reports (Moriarty et al., 1988; Sieja et al., 2005; von Mach-Szczypinski et al., 2009). In addition, significantly increased histamine levels were detected in the tumor-cell-conditioned medium (TCM) derived from all the examined murine tumor cell lines and human breast cancer cell lines compared with normal culture medium and control medium from the normal human breast epithelial cell line MCF-12A (FIG. 9J), suggesting that cancer cells may be a major source of increased histamine detected in tumor-bearing mice and cancer patients (FIGS. 2H and 9H). Consistently, increased expression of HDC, the histamine-synthesizing enzyme, was also detected in patients' breast cancer cells (FIG. 9K; data not shown, see FIG. S2 of U.S. Provisional Application No. 63/299,736). These data indicate that both histamine and HRH1 are upregulated in the immunosuppressive TME.

### Inhibition of HRH1 on Macrophages Restores T Cell Antitumor Immunity

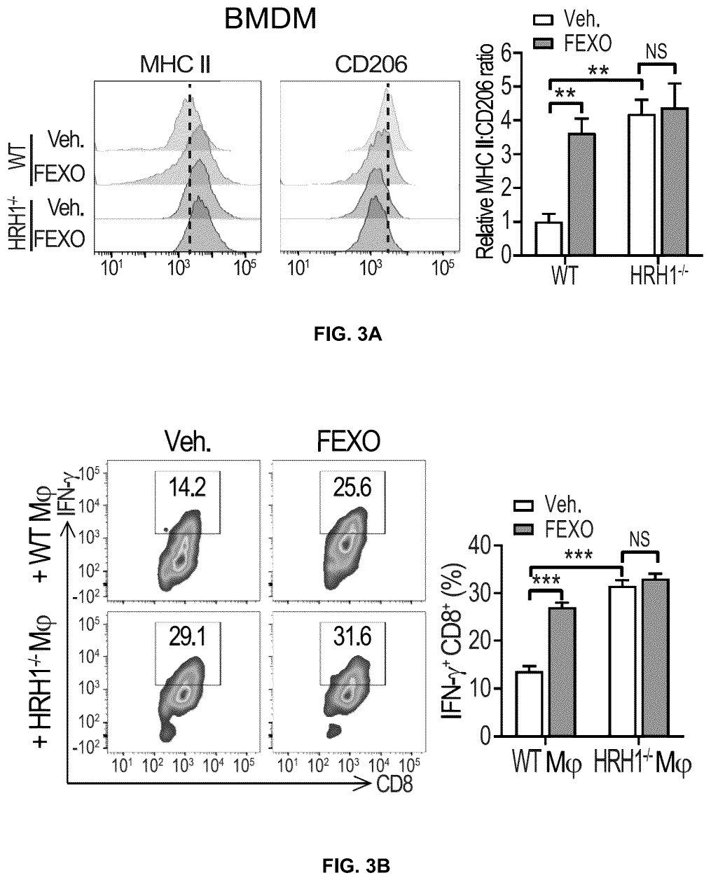

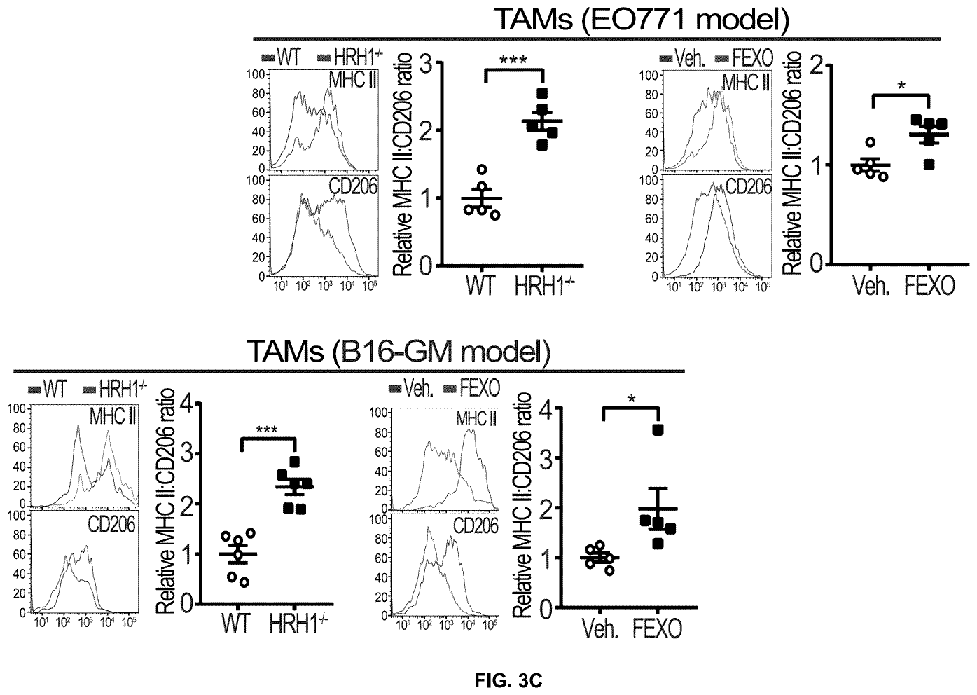

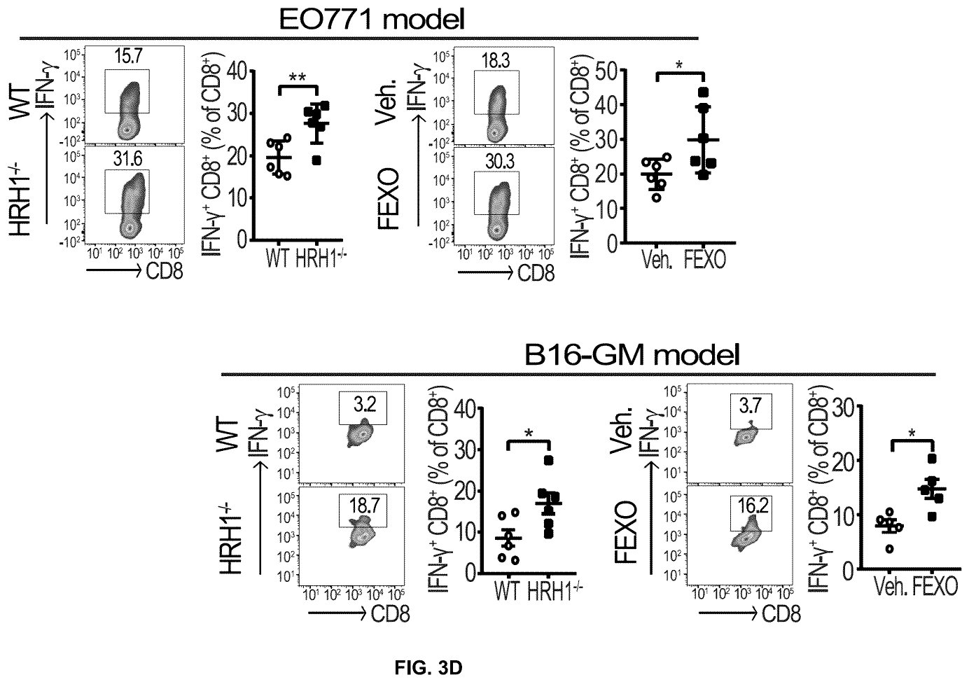

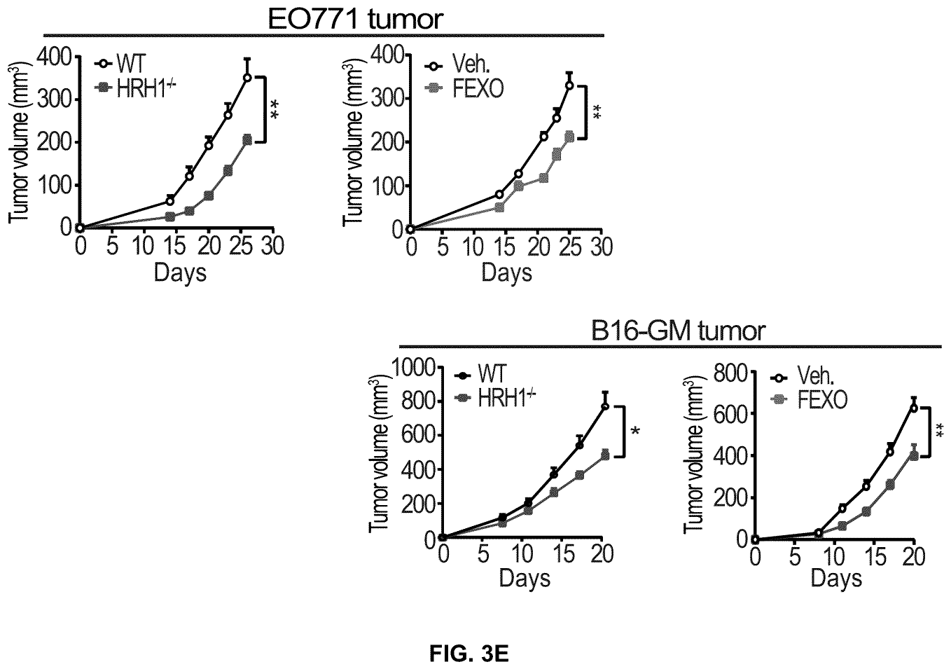

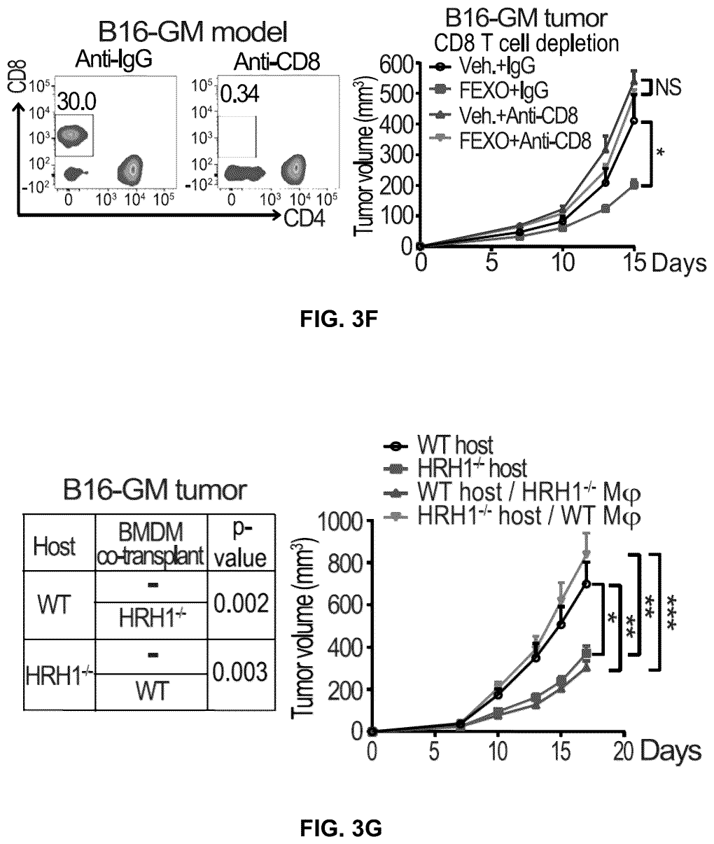

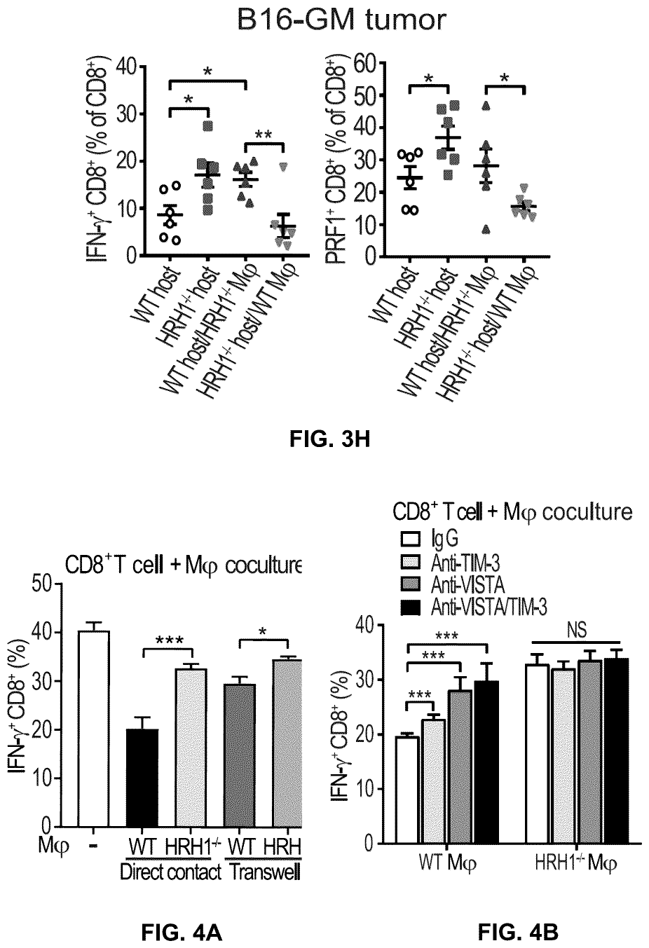

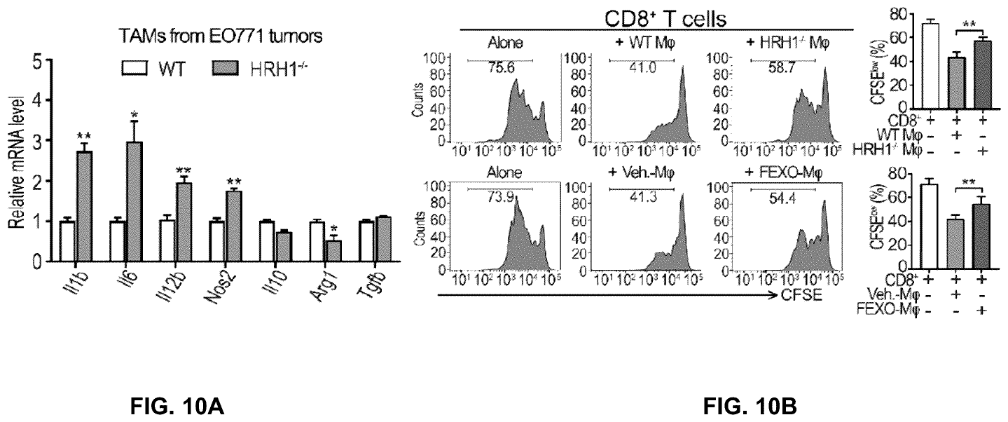

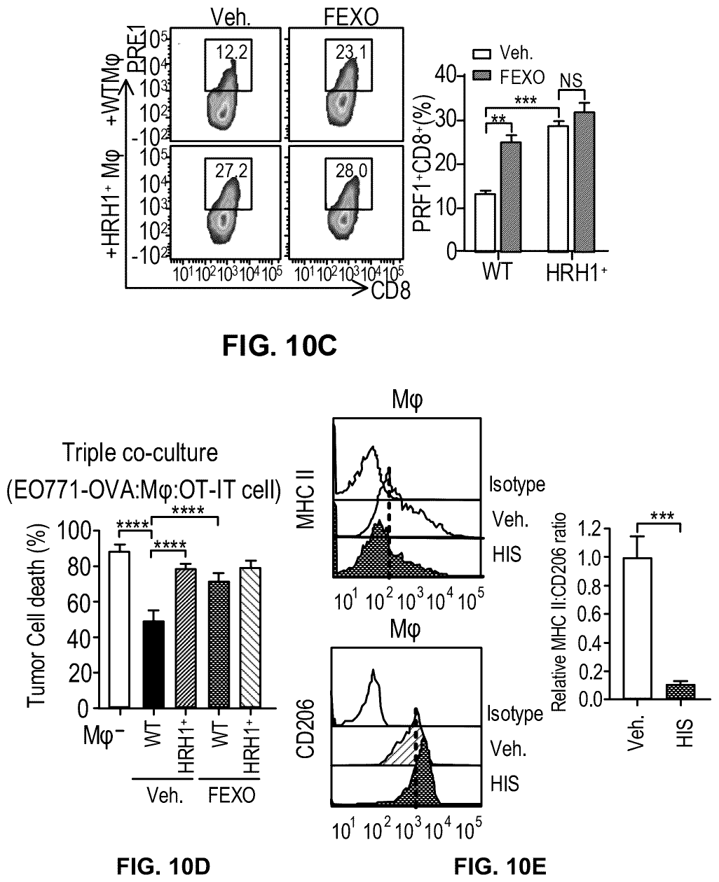

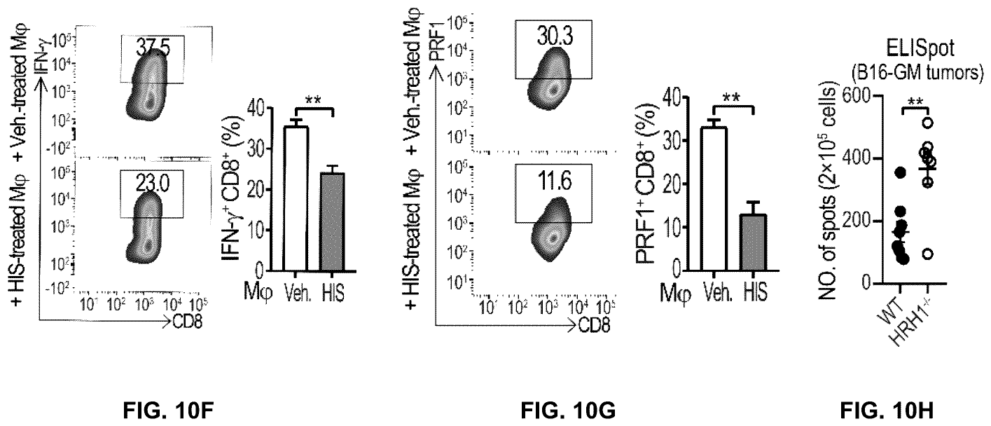

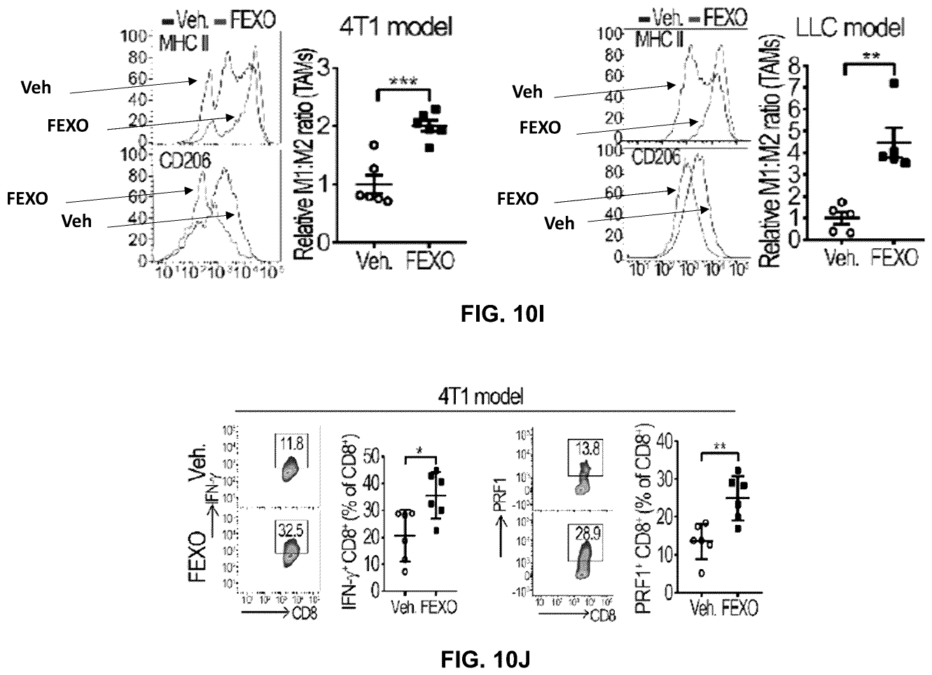

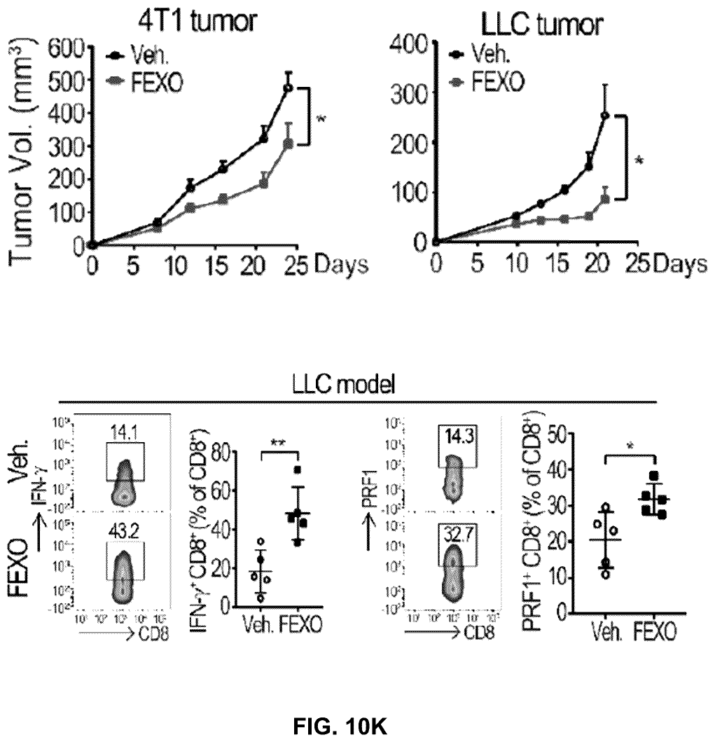

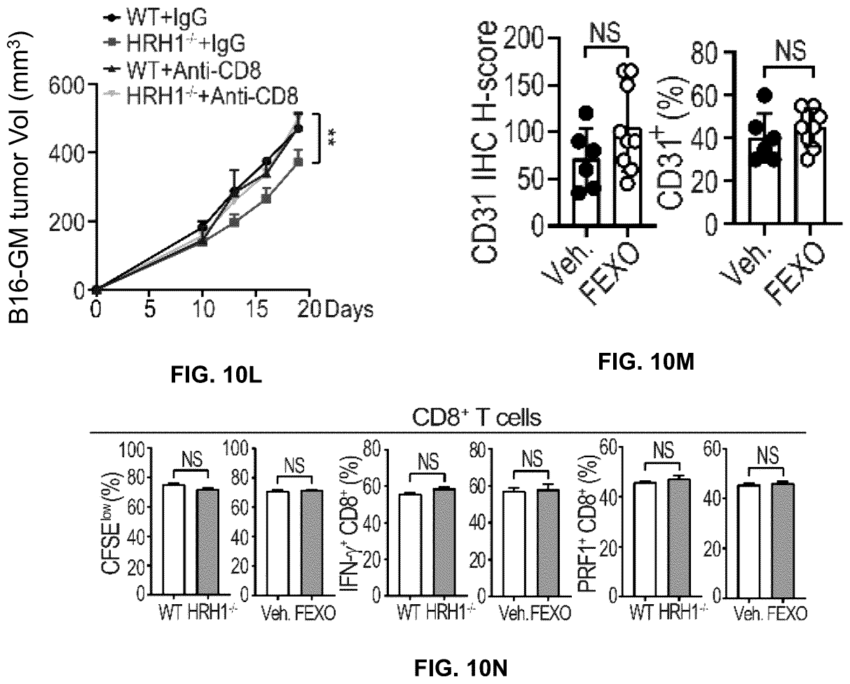

To investigate the specific function of the histamine-HRH1 axis in macrophages, we generated bone marrow-derived macrophages (BMDMs) from wild-type (WT) and HRH1-knockout (HRH1−/−) mice and treated them with TCM. Alternatively, we added an H1-antihistamine (fexofenadine, abbreviated as FEXO) to TCM-treated WT BMDMs. The expression ratio of major histocompatibility complex class II (MHCII, an M1 marker) versus CD206 (an M2 marker) was used to evaluate M1-M2 polarization status, which generally denotes antitumor versus protumor activities of TAMs, although it does not fully reflect TAMs' complexity in the TME (Guerriero, 2018). Both HRH1−/− and FEXO treatment polarized macrophages toward an M1-like phenotype characterized by increased MHCII and decreased CD206 (FIG. 3A). TAMs isolated from EO771 mammary tumors in HRH1−/− mice also had upregulated M1-like proinflammatory molecules (Il1b, Il6, Il12b, and No9) and attenuated M2-like marker Arg1 compared with those in WT mice (FIG. 10A). To examine the impact of macrophage HRH1 blockade on T cell activation, HRH1−/− or FEXO-treated macrophages were cultured with WT splenic T cells. HRH1−/− or FEXO treatment abrogated TAM-mediated T cell suppression, as signified by enhanced T cell proliferation; upregulated cytotoxic and cytolytic effector molecules, including IFN-g and perforin-1 (PRF1); and increased ovalbumin (OVA)-specific OT-I cell-mediated killing of OVA-transduced EO771 tumor cells (FIGS. 3B and 10B-10D). Notably, FEXO treatment of HRH1−/− macrophages did not increase the MHCII:CD206 ratio (FIG. 3A) or T cell activation/killing compared with vehicle-treated HRH1−/− macrophages (FIGS. 3B, 10C, and 10D), indicating that FEXO's effects on macrophages are mediated by HRH1. Conversely, histamine (10 mM)-treated mouse macrophages had increased M2-like marker CD206 and reduced M1-like marker MHCII compared with vehicle-treated ones (FIG. 10E). Importantly, histamine-treated macrophages significantly suppressed T cell activation compared with vehicle-treated macrophages (FIGS. 10F and 10G).

Next, we investigated the impact of histamine-HRH1 axis on TAMs and T cell immunity using two syngeneic tumor models in vivo. The EO771 mammary tumor cells or B16-GM (denotes B16-GM-CSF tumors with high tumor-infiltrating TAMs) melanoma cells (De Henau et al., 2016) were inoculated orthotopically into HRH1−/− mice and WT C57BL/6 mice. In separate experiments, WT mice were transplanted with these two cell lines and treated with vehicle or FEXO. Enhanced MHCII:CD206 ratio in the TAMs, increased numbers of IFN-g+ and PRF1+ CD8+ T cells, and reduced tumor growth were found in HRH1−/− mice and FEXO-treated mice compared with WT mice and vehicle treated mice, respectively (FIGS. 3C-3E; PRF1 data not shown). Furthermore, enzyme-linked immune absorbent spot (ELISPOT) assay revealed that tumor-reactive T cells were increased in tumors from HRH1−/− mice compared with those from WT mice (FIG. 10H). Similar changes were also detected in 4T1 mammary tumors or LLC lung carcinoma tumors in FEXO treated mice compared with vehicle-treated mice (FIGS. 10I-10K). The inhibition of B16-GM tumor growth in both HRH1−/− mice and FEXO-treated WT mice were blocked by depleting CD8+ T cells with anti-CD8 antibodies (FIGS. 3F and 10L), indicating that the enhanced antitumor activities by HRH1 blockade depend on CD8+ T cells. Although HRH1 was also expressed on endothelial cells (Lu et al., 2010), FEXO treatment did not show a significant impact on angiogenesis in EO771 tumors (FIG. 10M). To test whether HRH1 expressed on CD8+ T cells contributes to their biological functions, we compared HRH1−/− with WT T cells and FEXO-treated with vehicle-treated T cells, and found that they had similar proliferation rates and activities (FIG. 10N), suggesting that increased T cell activation by blocking HRH1 in mice was unlikely to have resulted from direct inhibition of HRH1 on CD8+ T cells.

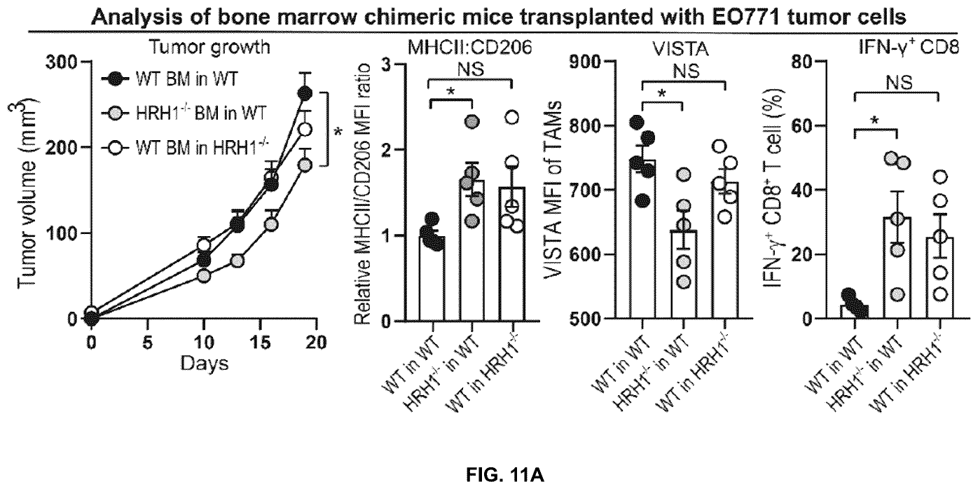

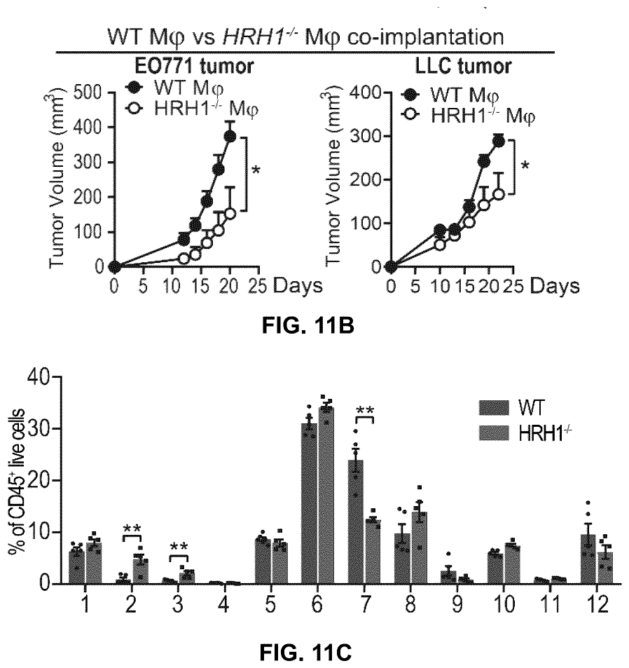

To explore whether loss of HRH1 expression on non-immune cells in the TME may also contribute to tumor suppression in HRH1−/− mice, we generated chimeric mice by transplanting WT bone marrow into HRH1−/− mice (WT BM in HRH1−/−) or HRH1−/− bone marrow into WT mice (HRH1−/− BM in WT) following lethal irradiation. One month later, the mice were orthotopically inoculated with EO771 tumor cells. HRH1−/− BM in WT mice showed decreased tumor growth along with increased MHCII:CD206 ratio in TAMs and increased IFN-g+ CD8+ T cell infiltration compared with that of control WT mice reconstituted with WT bone marrow cells (WT BM in WT) (FIG. 11A). Interestingly, WT BM in HRH1−/− mice seemed to have a minor tumor reduction, although statistically insignificant (FIG. 11A). To further determine the critical function of HRH1 on macrophages, we co-implanted WT or HRH1−/− macrophages with various types of cancer cells into recipient mice. Co-implantation of HRH1−/− BMDMs with B16-GM melanoma cells into WT mice significantly increased the activity of tumor-infiltrating CD8+ T cells and reduced tumor growth, which phenocopied the B16-GM tumors implanted in the HRH1−/− mice, whereas co-implanting WT BMDMs with B16-GM cells into HRH1−/− hosts enhanced tumor growth and suppressed CD8+ T cell activity, similar to the B16-GM tumors in WT mice (FIGS. 3G and 3H). Similarly, co-implanting HRH1−/− BMDMs with EO771 mammary tumor and LLC lung tumor cells into WT mice also significantly enhanced antitumor immunity and reduced tumor growth (FIG. 11B, data not shown). Together, these data demonstrated that HRH1 activation on macrophages suppresses CD8+ T cell activity and promotes tumor growth.

To evaluate the general impact of HRH1 blockade on the tumor immune microenvironment, we profiled CD45+ immune cells isolated from EO771 tumors grown in WT and HRH1−/− mice using mass cytometry (CyTOF), which revealed 12 distinct subsets, or clusters, of cells (data not shown, see FIG. S4C-S4E of U.S. Provisional Application No. 63/299,736). EO771 tumors from HRH1−/− mice had significantly fewer M2-like macrophages (cluster 7), whereas cytotoxic immune cells, including CD8+ T cells (cluster 2), were increased in tumors from HRH1−/− mice (FIG. 11C; data not shown, see FIG. 3I of U.S. Provisional Application No. 63/299,736), suggesting enhanced antitumor immunity. Moreover, the MHCII:CD206 ratio of TAMs was increased, along with GZMB+ CD8+ T cells, in HRH1−/− mice compared with WT mice (, see FIG. S4G of U.S. Provisional Application No. 63/299,736).

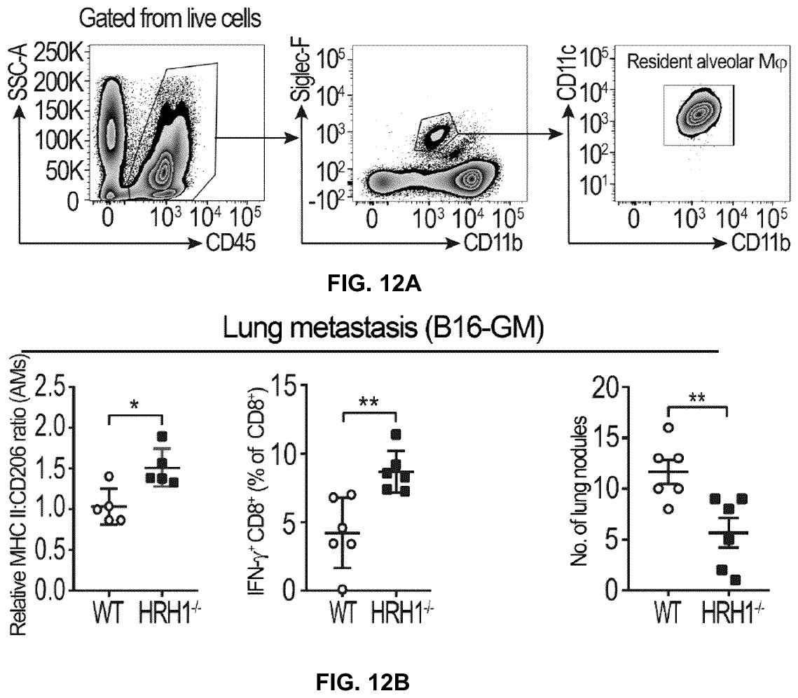

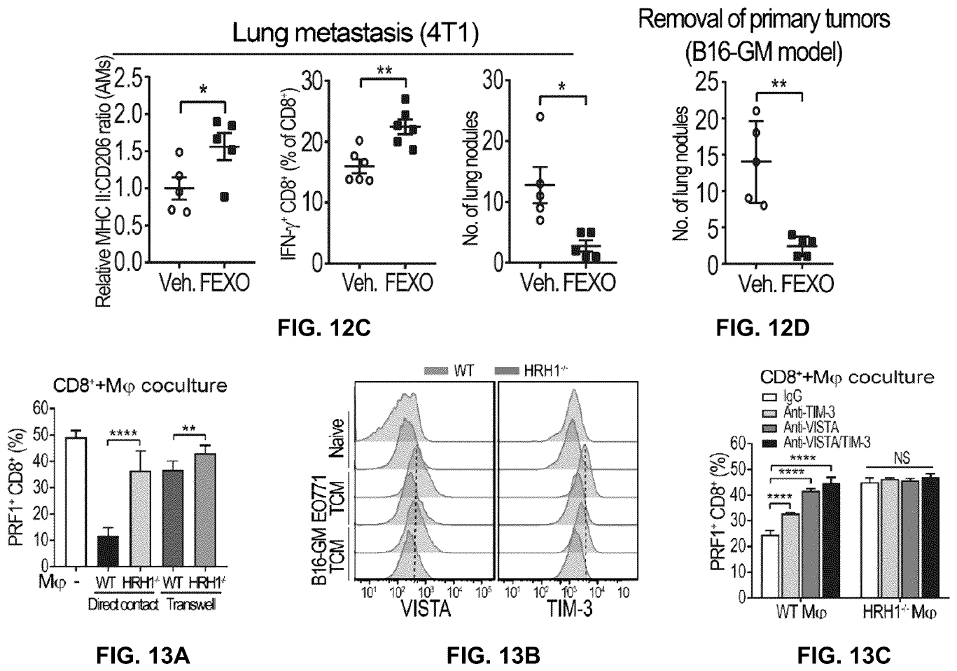

HRH1 blockade also enhanced antitumor immunity in lung metastatic sites of two spontaneous lung metastasis models, B16-GM and 4T1, as shown by increased M1-like polarization of resident alveolar macrophages (Misharin et al., 2013), increased cytotoxic CD8+ T cells, and reduced lung metastases (FIGS. 12A-12C). B16-GM lung metastases in FEXO-treated mice were also inhibited compared with vehicle-treated mice after B16-GM primary melanomas were surgically removed upon growing to a tumor size of 100 mm3 (FIG. 12D), indicating that antihistamines enhanced antimetastasis immune response.

### HRH1 Activation Promotes VISTA Membrane Localization

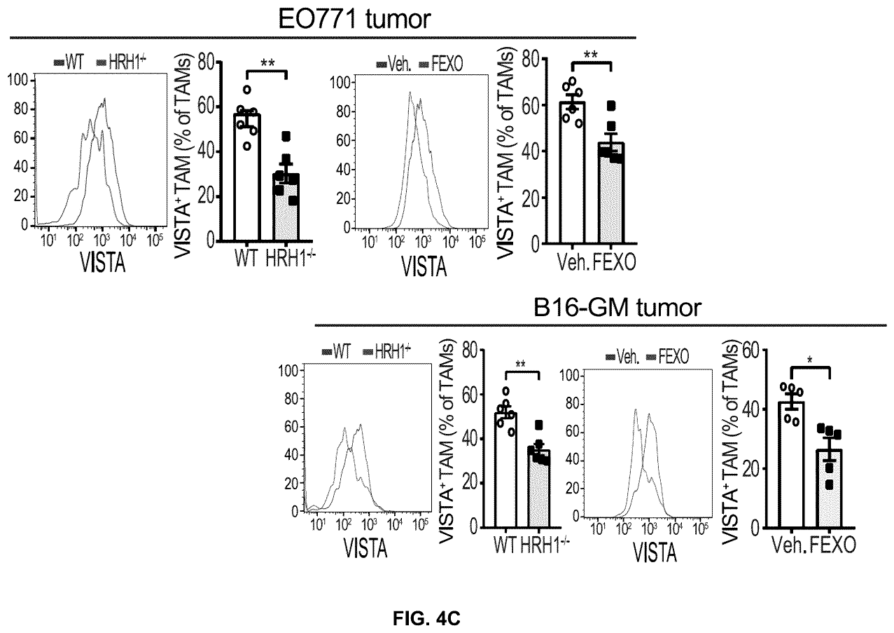

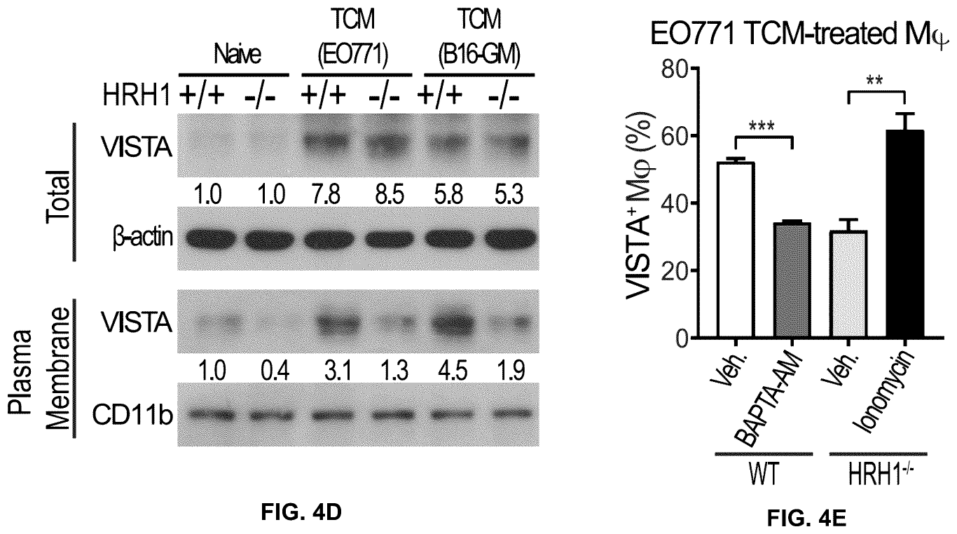

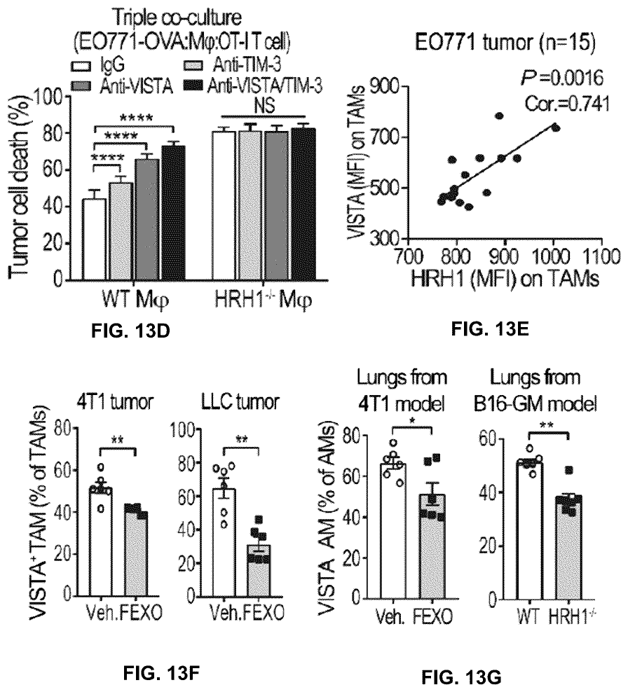

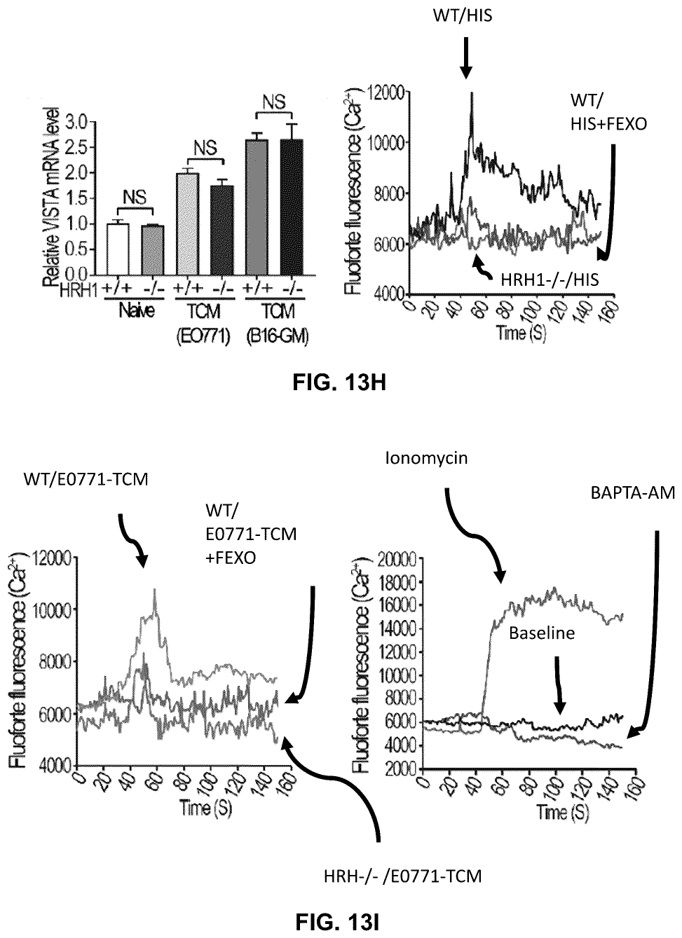

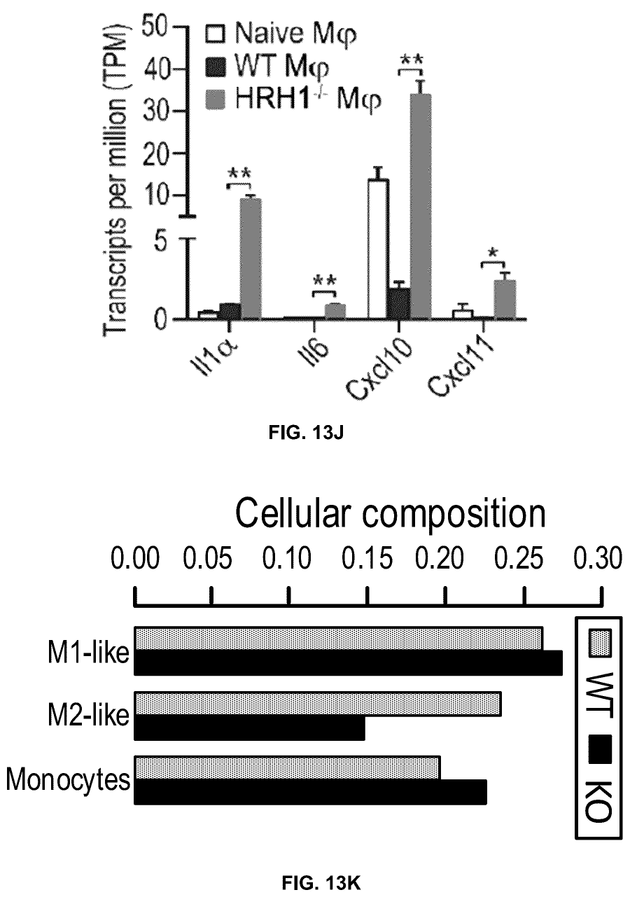

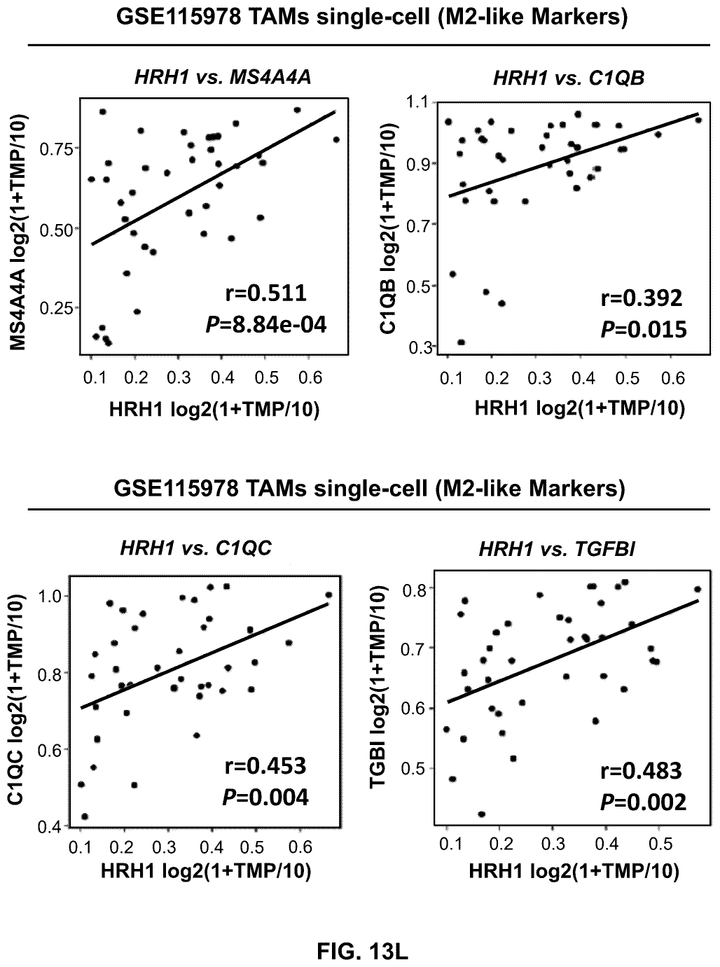

To explore how HRH1 on macrophages suppresses T cell activities, EO771 TCM-treated WT or HRH1−/− macrophages were co-cultured with WT CD8+ T cells in direct contact or separately in a transwell. Modulation of IFN-g+ PRF1+ CD8+ T cells by macrophage HRH1 was largely dependent on direct cell-cell contact (FIGS. 4A and 13A). Since macrophages and dendritic cells can regulate T cell function via engagement of co-stimulatory or inhibitory receptors on T cells (Guerriero, 2019; Ostuni et al., 2015), we investigated whether HRH1 on macrophages induces T cell dysfunction via regulating co-stimulatory or inhibitory receptors on T cells. Among the 13 ligands with costimulatory or inhibitory activities screened on EO771 or B16-GM TCM-treated macrophages, VISTA and TIM-3, known inhibitory molecules (Lines et al., 2014; Ocana-Guzman et al., 2016), were the most downregulated molecules on HRH1v macrophages compared with WT macrophages (FIG. 13B). Functionally, when WT macrophages in TCM were pre-treated with VISTA-blocking antibody and co-cultured with T cells, IFN-g+ and PRF1+ CD8+ T cell levels and tumor cell killing activity increased to levels similar to those of the T cells cocultured with HRH1/macrophages; a TIM-3-blocking antibody had a lesser effect (FIGS. 4C, 13C, and 13D), suggesting that VISTA is a major HRH1 downstream mediator of T cell dysfunction. In EO771 tumors from WT mice, HRH1 expression on TAMs was strongly correlated with VISTA expression (FIG. 13E). Reduced VISTA expression was detected on TAMs from EO771 and B16-GM tumors in HRH1−/− mice or FEXOtreated WT mice compared with respective controls (FIG. 4D). Decreased VISTA on TAMs was also observed in FEXO-treated other tumor models (e.g., 4T1 and LLC) in WT BALB/c or B6 mice compared with that of vehicle-treated ones (FIG. 13F). Similarly, VISTA expression on alveolar macrophages from lung metastases of HRH1−/− mice bearing B16-GM tumor or of FEXO-treated WT mice bearing 4T1 tumor was also downregulated compared with their controls (FIG. 13G).

Blocking HRH1 significantly reduced VISTA membrane expression on macrophages (FIGS. 4D and 4E) but VISTA mRNA and total protein expression did not change significantly (FIGS. 4E and 13H). Cell fractionation analysis confirmed that HRH1 blockade decreased cell membrane VISTA protein expression (FIG. 4E). Calcium (Ca2+) facilitates protein trafficking to the plasma membrane, and HRH1 activation induces Ca2+ release from the endoplasmic reticulum (Micaroni, 2010, 2012; Parsons and Ganellin, 2006). Thus, we explored whether HRH1 activation may foster VISTA membrane trafficking via releasing Ca2+. Indeed, after histamine or TCM treatment that induced activation of HRH1, intracellular free Ca2+ levels were higher in WT macrophages than in HRH1−/− macrophages and FEXO-treated WT macrophages (FIG. 13I). Blocking Ca2+ flux with BAPTA-AM, an intracellular calcium chelator, reduced HRH1-mediated membrane VISTA expression on WT TAMs, while the Ca2+ flux agonist ionomycin increased cell-surface VISTA expression on HRH1−/− TAMs (FIGS. 4F and 13I). These data suggest that HRH1-modulated Ca2+ release is critical for VISTA membrane localization.

### HRH1 Activation Reshapes the Transcriptomic Landscape of Macrophages

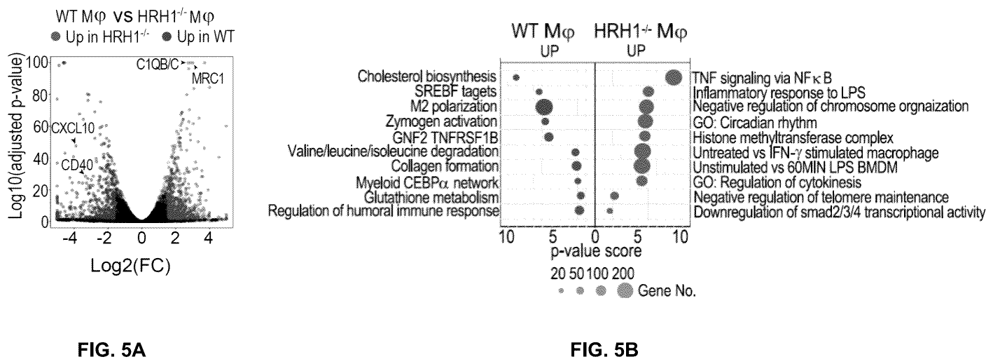

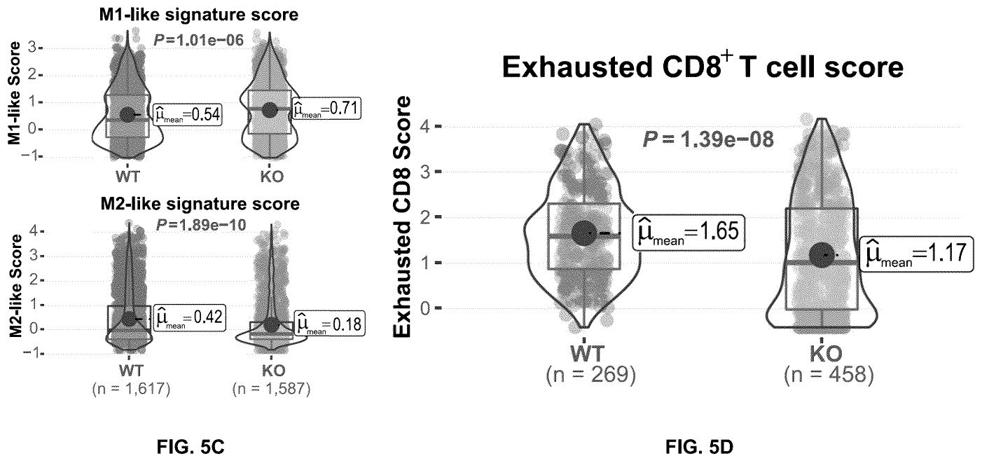

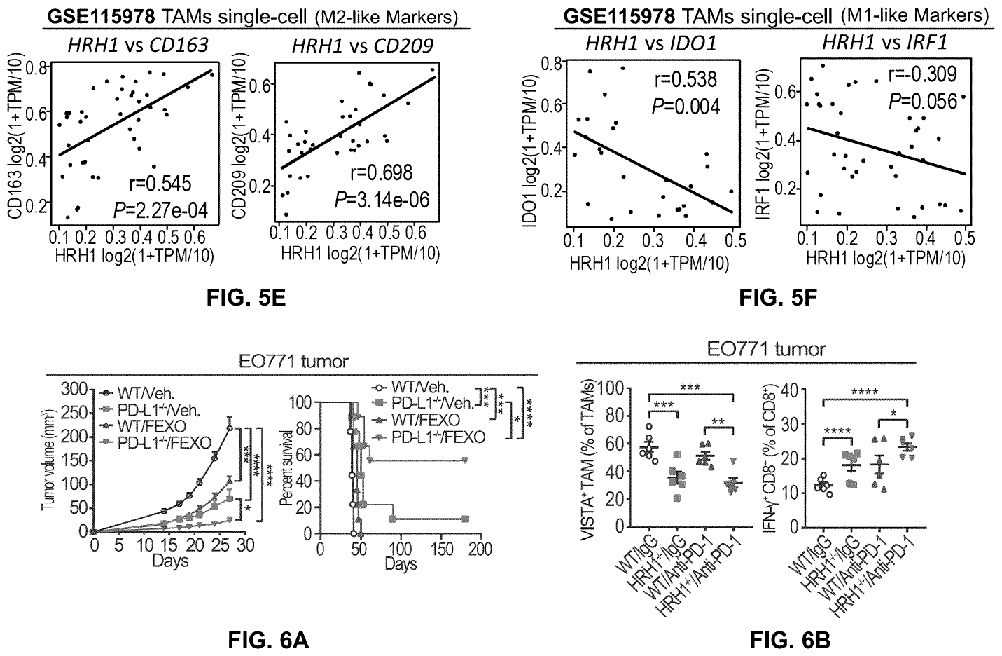

To gain deep insight into HRH1 downstream signaling that may contribute to the immunosuppressive phenotype of macrophages, we profiled the global transcriptome of TCM-treated WT and HRH1−/− macrophages by RNA sequencing. Compared with WT macrophages, HRH1−/− macrophages showed higher expression of genes associated with M1 polarization (e.g., CXCL10 and CD40) but lower expression of many genes associated with M2-like phenotype (e.g., C1QB, C1QC, and MRC1) (FIG. 5A). Gene set enrichment analysis (GSEA) identified key canonical pathways specifically up- or downregulated in HRH1−/− macrophages compared with WT macrophages (FIG. 5B). For example, TCM-treated HRH1−/− macrophages showed significantly upregulated TNF-a signaling and lipopolysaccharide (LPS)-stimulated signaling and IFN-g signaling (FIG. 5B). Various proinflammatory cytokines and chemokines (e.g., Il6, Il1a, Cxcl10, and Cxcl11) are also significantly higher in HRH1−/− macrophages than in WT macrophages (FIG. 13J). These upregulated signaling pathways and molecules in HRH1−/− macrophages are tightly associated with M1 polarization of macrophages and antitumor immune reactivity of macrophages (DeNardo and Ruffell, 2019); conceivably, they may contribute to the increased anti-tumor activities of HRH1−/− macrophages. On the other hand, reduced M2-polarized macrophage signature (Gerrick et al., 2018) was detected in HRH1−/− macrophages (FIG. 5B), consistent with their M1-like polarization phenotype. Intriguingly, cholesterol biosynthesis and targets of sterol regulatory element binding transcription factors (SREBF1/2) are also among the inhibited signaling pathways in HRH1−/− macrophages compared with WT macrophages (FIG. 5B).

To further explore the broad impact of HRH1 blockade on macrophage phenotype and the landscape of the tumor immune microenvironment in vivo, the CD45+ immune cells were isolated from EO771 tumors growing in WT versus HRH1−/− mice for single-cell RNA sequencing (scRNA-seq) analyses. Major immune cell types were predicted using built-in annotated human peripheral blood monocyte (PBMC) datasets as a reference, and the automatic cell annotations were further calibrated by examining the most highly expressed marker genes between clusters (; data not shown, see FIG. 5C of U.S. Provisional Application No. 63/299,736). The data showed that HRH1−/− primarily affected TAMs and T cells among the CD45+ immune cells. Cell composition analysis showed reduced M2-like macrophages and slightly increased M1-like macrophages in tumors from HRH1−/− mice compared with those from WT mice (; data not shown, see FIG. S6K of U.S. Provisional Application No. 63/299,736). We also calculated M1 and M2 gene signature-based scores at the single-cell level in TAMs. Overall, macrophages isolated from EO771 tumors in HRH1−/− mice showed significantly higher M1-gene signature scores, but much lower M2-gene signature scores compared with macrophages from WT mice (FIG. 5C). Since our above studies suggested a correlation between HRH1 expression and T cell dysfunction (FIGS. 1F, data not shown, see FIGS. S1F-S1H of U.S. Provisional Application No. 63/299,736), we further evaluated exhausted CD8+ T cell gene signature scores at the single-cell level in CD8+ T cells isolated from the tumors. Indeed, CD8+ T cells isolated from EO771 tumors in HRH1−/− mice showed much lower exhausted CD8+ T cell gene signature scores compared with those from the WT mice (FIG. 5D), indicating reduced T cell dysfunction by HRH1 loss.

To validate the above findings in cancer patients, we further analyzed correlations between HRH1 and human M1- and M2-macrophage markers at the single-cell level in TAMs collected from melanoma patients (GSE115978) (Jerby-Arnon et al., 2018). We found that HRH1 strongly and positively correlated with well-known human M2-macrophage markers (Martinez et al., 2006), e.g., CD163, CD209, and C1QB/C, at the single-cell level (FIGS. 5F and 13). On the other hand, HRH1 negatively correlated with human M1-macrophage markers, including IRF1 and IDO1, both of which are downstream of IFN-g signaling (FIG. 5F).

Taken together, these results show that blocking HRH1 reshaped the transcriptomic landscape of immune cells, among which reduced M2-like macrophage signatures and enhanced cytotoxic T cell functions mostly contribute to the alleviation of immunosuppression in TME.

### HRH1 Inhibition Enhances Therapeutic Responses to ICB

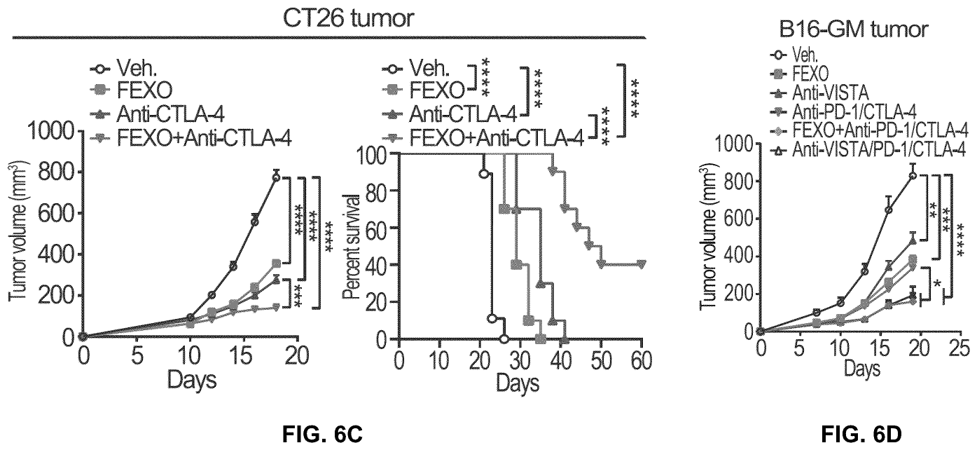

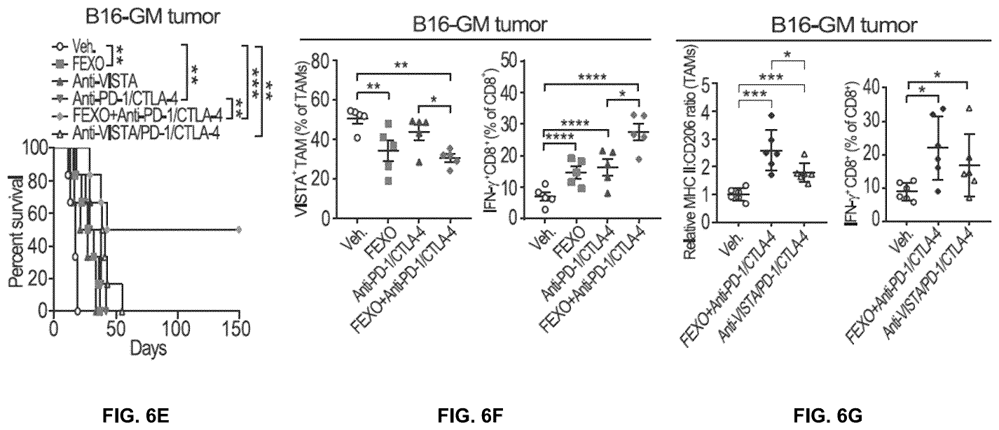

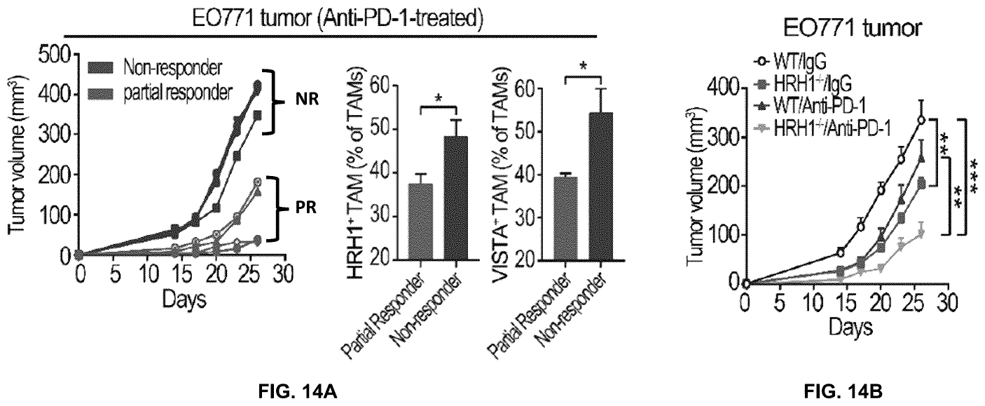

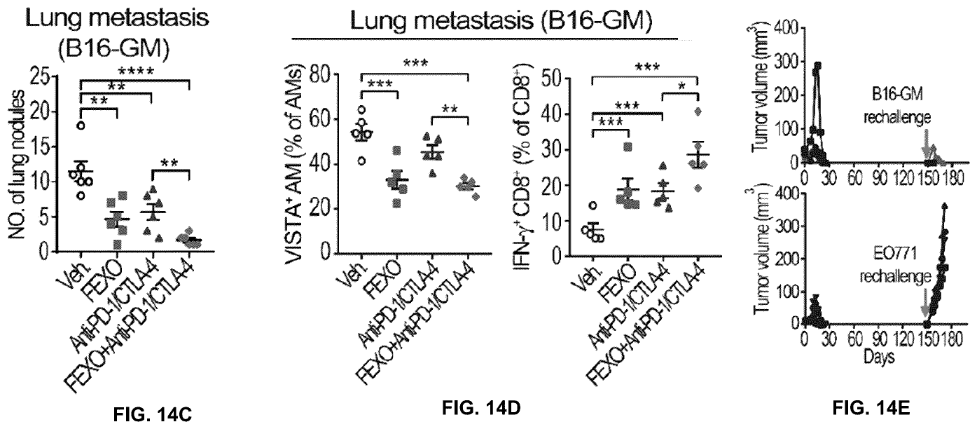

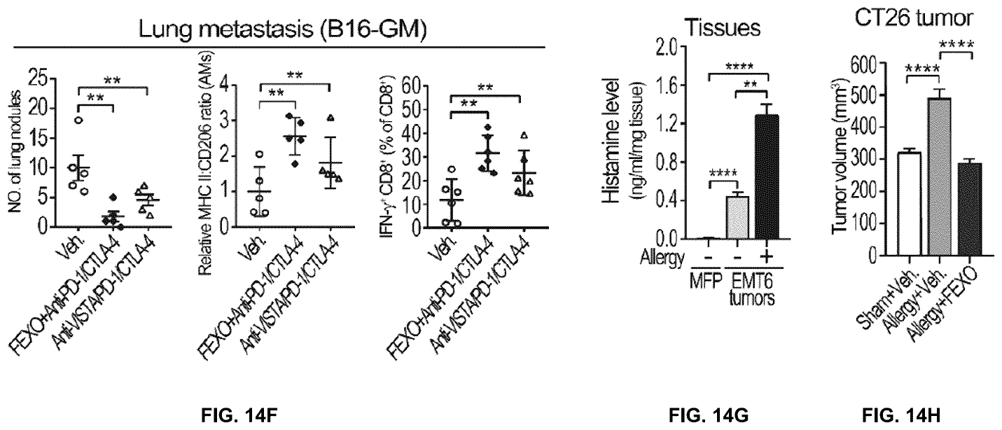

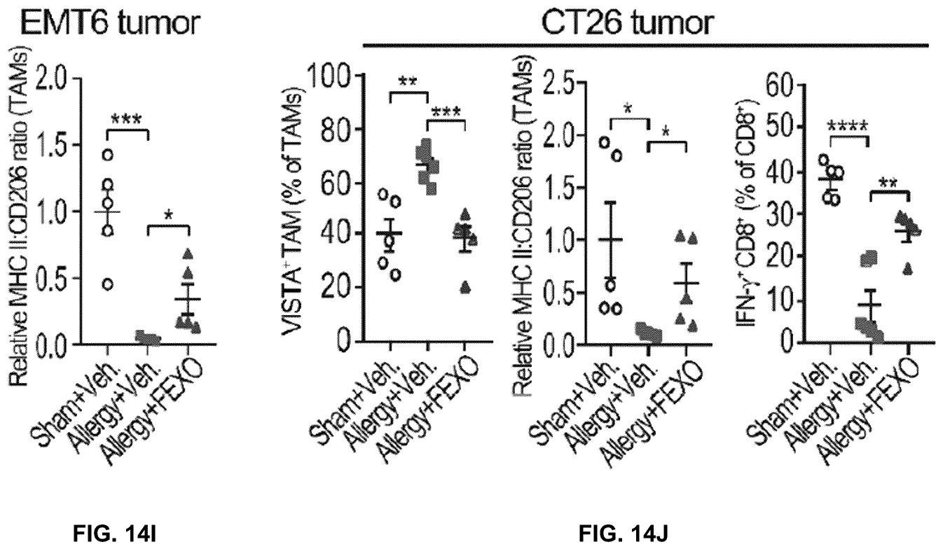

HRH1 activation promotes VISTA membrane localization (FIG. 4E), and patients with high HRH1-expressing tumors showed poor responses to anti-PD-1 immunotherapy (FIG. 1G). VISTA and PD-1/PD-L1 suppress T cell activity non-redundantly, and upregulation of VISTA has been linked with ICB resistance in cancer patients (Blando et al., 2019; Gao et al., 2017; Liu et al., 2015; Nowak et al., 2017). Therefore, we further investigated whether high HRH1 expression, via induction of VISTA membrane expression, would confer immunotherapy resistance and HRH1 blockade could enhance response to immunotherapy. We found that, among EO771 tumors in mice having heterogeneous responses to anti-PD-1 treatment, non-responding tumors had higher HRH1 and VISTA expression on TAMs than did partially responding tumors (FIG. 14A). To test if inhibition of HRH1 would enhance antitumor activity of PD-L1 blockade, EO771 tumors in WT or PD-L1−/− mice were treated by vehicle or FEXO. FEXO-treated PD-L1−/− mice showed the most effective tumor inhibition and dramatically prolonged survival (FIG. 6A), with 50% of mice remaining tumor free, whereas only 10% of vehicle-treated PD-L1−/− mice were tumor free. Similarly, growth of anti-PD-1-treated EO771 tumor in HRH1−/− mice was effectively inhibited, accompanied by lower VISTA membrane expression on TAMs and more IFN-g+ CD8+ T cells compared with anti-PD-1-treated WT mice (FIGS. 6B and 14B). Next, we examined whether inhibiting HRH1 activation with antihistamine could also enhance therapeutic efficacy of anti-CTLA-4 immune checkpoint inhibitor. FEXO or anti-CTLA-4 treatment both delayed tumor growth in the CT26 murine colorectal carcinoma model, and combinatorial treatment of FEXO plus anti-CTLA-4 more effectively inhibited CT26 tumor growth (FIG. 6C, left). Remarkably, FEXO plus anti-CTLA-4 combinatorial treatment resulted in complete tumor remission and tumor-free survival in 40% of the mice, while none of the mice in other groups survived by day 41 post-injection (FIG. 6C, right). Furthermore, in the ICB-resistant B16-GM melanoma model, FEXO treatment combined with ICB (anti-PD-1 plus anti-CTLA-4) achieved the highest therapeutic response and drastically inhibited both primary tumor growth and lung metastasis compared with FEXO or ICB alone (FIGS. 6D, 6E, and 14C). Complete tumor remission was observed in 50% of the FEXO+ICB combination treatment group but in none of the other groups (FIG. 6E), along with significantly downregulated VISTA expression on TAMs and enhanced T cell function at both primary and metastatic tumor sites (FIGS. 6F and 14D). Next, the FEXO+ICB-treated B16-GM tumor-free mice were reinoculated with B16-GM cells or EO771 cells. B16-GM cells, not EO771 cells, were rejected, indicating a persistent T cell memory for B16-GM tumor cells (FIG. 14E). Together, the results show that HRH1 knockout or antihistamines combined with ICB greatly improved the therapeutic response of multiple tumor models, echoing our clinical findings that cancer patients who took H1-antihistamines during ICB treatments had better overall survival (FIG. 1B).

VISTA-blocking antibodies are currently being tested in clinical trials for antitumor efficacy (Nowak et al., 2017). We tested whether FEXO, a low-cost OTC drug, may have effects similar to those of anti-VISTA antibodies. FEXO monotherapy showed similar antitumor activity compared with VISTA antibodies in the B16-GM model (FIG. 6D). When combined with ICB therapy, FEXO and the anti-VISTA antibodies also had similar efficacy in controlling primary tumor growth (FIG. 6D). Amazingly, FEXO+ICB was more effective than anti-VISTA+ICB for prolonging survival of mice, because 50% of FEXO+ICB-treated mice had tumor-free survival but none of the anti-VISTA+ICB treated mice survived (FIG. 6E). FEXO+ICB was also more potent than anti-VISTA+ICB in promoting macrophage e M1-like polarization and inhibiting lung metastasis (FIGS. 6G and 14F).

Allergies Induce Immunotherapy Resistance, which is Mitigated by HRH1 Blockade

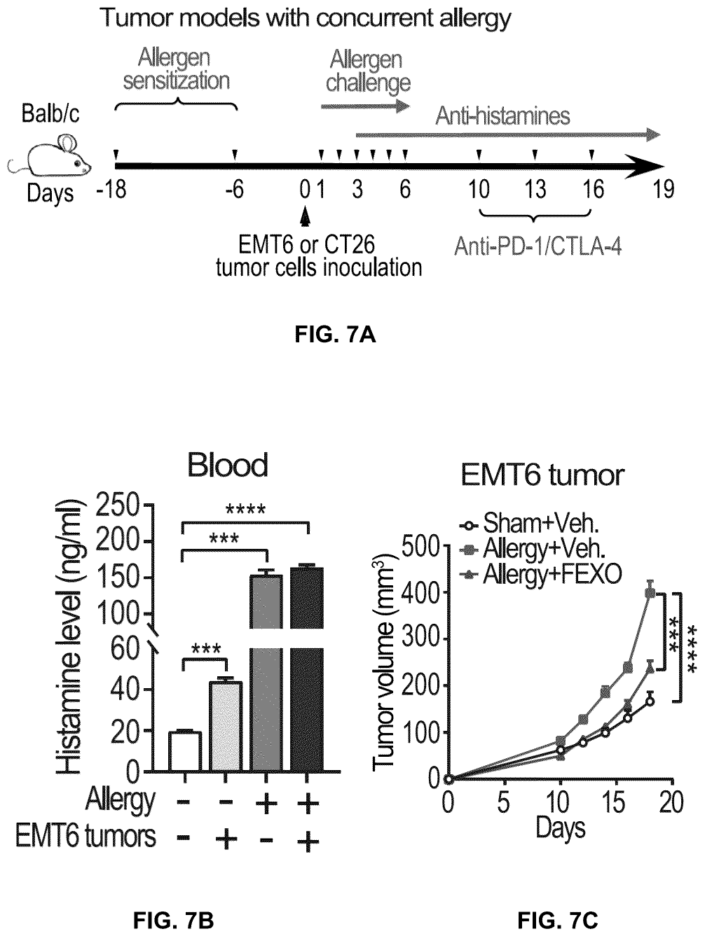

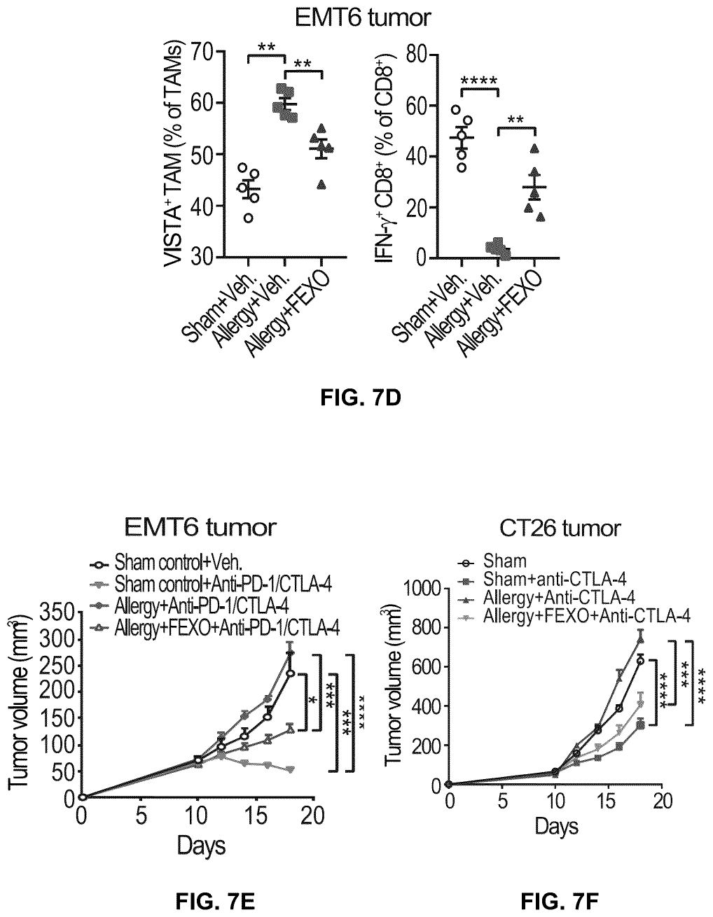

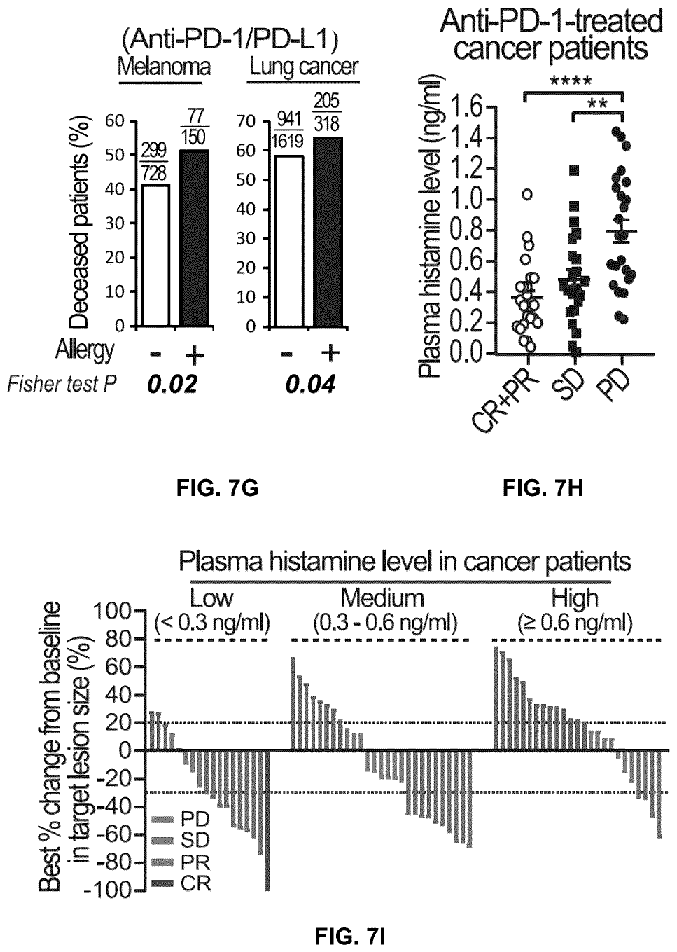

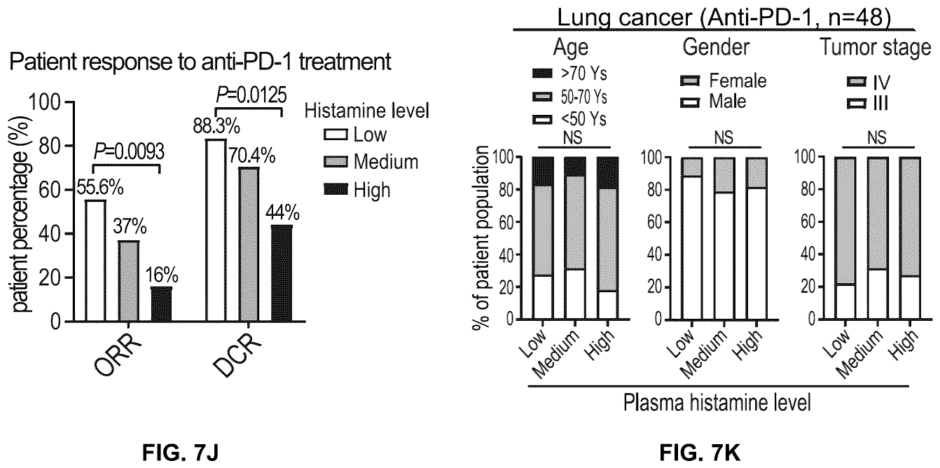

The above studies indicate that histamine-HRH upregulation in TME induces T cell dysfunction and immunotherapy resistance. Since allergic reactions release lots of histamine, we questioned whether allergy similarly influences antitumor immunity and immunotherapy response. To address the question, an OVA induced allergic airway disease model (Nials and Uddin, 2008), in which BALB/c mice had two rounds of allergen (OVA) sensitization, was transplanted with tumor cells, followed by 1 week of airway OVA allergen exposure and treatment with FEXO, ICB, or FEXO+ICB (FIG. 7A). To study allergy's impact on tumor immunity and immunotherapy, we used two murine tumor models, EMT6 (mammary tumor) and CT26 (colon cancer), both of which were derived from the BALB/c background, which is susceptible to OVA-induced allergy (Kumar et al., 2008). High levels of histamine in plasma and tumor tissues were detected in mice after exposure to OVA (FIGS. 7B and 14G), indicating allergic reaction to OVA. Compared with the sham control group, mice with an OVA-induced allergic response had significantly accelerated EMT6 tumor growth, which was largely blocked by FEXO treatment (FIG. 7C). Similarly, tumor growth of CT26 colon cancer cells in OVA-allergic BALB/c mice was significantly increased compared with that in sham control mice and was largely blocked by FEXO treatment (FIG. 14H). Both EMT6 or CT26 tumors in allergic mice also had increased VISTA expression on TAMs, a decreased MHCII:CD206 ratio, and reduced IFN-g+ CD8+ T cells, all of which could be partially reversed by FEXO treatment (FIGS. 7D, 14I, and 14J).

EMT6 and CT26 tumors are relatively sensitive to ICB treatment, as seen in sham control mice (FIGS. 7E and 7F) and as previously reported (Khononov et al., 2021; Mosely et al., 2017). However, both EMT6 and CT26 tumors became completely resistant to ICB therapy in allergic mice (FIGS. 7E and 7F). Remarkably, FEXO treatment largely restored sensitivity of EMT6 and CT26 tumors to ICB therapy in allergic mice (FIGS. 7E and 7F). The data indicate that allergic reaction promotes cancer immune evasion and immunotherapy resistance via the histamine-HRH1 axis, and this immune evasion could be mostly blocked by antihistamines.

Next, to examine the clinical impact of allergic response on immunotherapy efficacy in cancer patients, we retrospectively analyzed survival data of melanoma and lung cancer patients who reported allergic reactions before receiving anti-PD-1/PDL1 treatment versus those who did not (Table S3). Indeed, cancer patients who experienced allergies had significantly worse outcomes compared with those patients who had no allergy (FIG. 7G, melanoma patients, 51% versus 41% deceased; lung cancer patients, 64% versus 58% deceased).

TABLE S3
Allergy information of cancer patients, related to FIG. 7.

| | Allergy status | No allergy status | |
|---|---|---|---|
| Patient Cancer type | reported | reported | Sum |
| Melanoma (2016-2017) | 150 | 728 | 878 |
| Breast Cancer (2016-2018) | 88 | 254 | 342 |
| Lung Cancer (2016-2018) | 318 | 1619 | 1937 |
| Colon Cancer (2016-2018) | 100 | 287 | 387 |

Last, we directly examined whether plasma histamine levels were associated with patients' response to immunotherapies. We measured pre-treatment histamine levels in plasma collected from a cohort of cancer patients (n=70) enrolled in a basket trial of anti-PD-1 treatment, which included lung cancer, breast cancer, and colon cancer patients. We found markedly lower levels of histamine in the blood of patients with complete response (CR) or partial response (PR) compared with those of patients with progressive disease (PD) (FIG. 7H). Patients with stable disease (SD) had plasma histamine levels lower than those of PD patients but higher than those of CR/PR patients (FIG. 7H). Next, we separated cancer patients into three groups based on their plasma histamine levels: patients with low levels of histamine (<0.3 ng/mL, which is the average level of histamine among healthy subjects; see FIG. 2H), medium levels of histamine (0.3-0.6 ng/mL), and high levels of histamine (>0.6 ng/mL) (Tables S4-S6). Patients with low levels of plasma histamine had more than tripled overall response rate (ORR) and doubled disease control rate (DCR) (ORR 55.6% versus 16%; DCR 88.3% versus 44%) compared with patients with high levels of plasma histamine (FIGS. 7I and 7J). Notably, there were no significant differences in age, gender, and tumor stage among the three groups (FIG. 7K; Tables S4-S6). These data from immunotherapy-treated cancer patients support the clinical relevance of our experimental findings from mouse tumor models with OVA-induced allergic airway disease (FIGS. 7E and 7F), suggesting that histamine release either from allergy response or by cancer cells attenuates response to immunotherapies, which can be mostly rescued by antihistamines.

TABLE S4
Responses of anti-PD-1-treated lung cancer patients (n = 48) and distributions of their
age, sex, and tumor stagewith indicated plasma histamine levels, related to FIGS. 7A-7K.

| | Treatment response | | | | Age (Years) | | | Gender | | TNM stage | |
|---|---|---|---|---|---|---|---|---|---|---|---|
| Histamine | CR | PR | SD | PD | >70 | 50-70 | <50 | Male | Female | III | IV |
| Low (<0.3 ng/ml) | 1 | 9 | 6 | 2 | 5 | 10 | 3 | 16 | 2 | 4 | 14 |
| Medium (0.3-0.6 ng/ml) | 0 | 7 | 6 | 6 | 3 | 14 | 2 | 15 | 4 | 6 | 13 |
| High (≥0.6 ng/ml) | 0 | 2 | 3 | 6 | 2 | 7 | 2 | 9 | 2 | 3 | 8 |

TABLE S5
Responses of anti-PD-1-treated colon cancer patients (n = 12) and distributions of their
age, sex, and tumor stage with indicated plasma histamine levels, related to FIGS. 7A-7K.

| | Treatment response | | | Age (Years) | | | Gender | | TNM stage | |
|---|---|---|---|---|---|---|---|---|---|---|
| Histamine | PR | SD | PD | >70 | 50-70 | <50 | Male | Female | III | IV |
| Low (<0.3 ng/ml) | 0 | 0 | 0 | 0 | 0 | 0 | 0 | 0 | 0 | 0 |
| Medium (0.3-0.6 ng/ml) | 2 | 1 | 1 | 2 | 0 | 2 | 4 | 0 | 1 | 3 |
| High (≥0.6 ng/ml) | 2 | 2 | 4 | 3 | 3 | 2 | 6 | 2 | 1 | 7 |

TABLE S6
Responses of anti-PD-1-treated breast cancer patients (n = 10) and distributions of
their age, sex, and tumor stage with indicated plasma histamine levels, related to FIG. 7.

| | Treatment response | | | Age (Years) | | | Gender | | TNM stage | |
|---|---|---|---|---|---|---|---|---|---|---|
| Histamine | PR | SD | PD | >70 | 50-70 | <50 | Male | Female | III | IV |
| Low (<0.3 ng/ml) | 0 | 0 | 0 | 0 | 0 | 0 | 0 | 0 | 0 | 0 |
| Medium (0.3-0.6 ng/ml) | 1 | 2 | 1 | 0 | 3 | 1 | 0 | 4 | 0 | 4 |
| High (≥ 0.6 ng/ml) | 0 | 2 | 4 | 1 | 4 | 1 | 0 | 6 | 1 | 5 |

### Discussion

In this study, we found that melanoma and lung cancer patients taking H1-antihistamines during immunotherapy treatment exhibited improved clinical outcomes with statistical significance. Similar trends were also observed in ICB-treated breast and colon cancer patients, although statistical significance was not achieved, which was likely due to the smaller patient numbers enrolled in the ICB treatment at the time compared with melanoma and lung cancer patients. These clinical data suggest that H1-antihistamines augment T cell-mediated antitumor immunity.

There were previous controversial reports on histamine modulation of myeloid-derived suppressive cells (MDSCs). Using different mouse and tumor models, some studies modulated histamine production and suggested that histamine reduced MDSCs and suppressed tumor growth (Grauers Wiktorin et al., 2019; Yang et al., 2011), while others found that histamine from mast cells increased MDSC proliferation and survival and promoted B16 melanoma metastasis (Martin et al., 2014). It is possible that different histamine concentrations and histamine receptors were inducing distinct effects on MDSCs. Antihistamines, when combined with chemotherapies, were reported to have either inhibitory or promoting effects on certain types of cancers, but antihistamines had not been tested for combination with any other therapies, especially not immunotherapy, for cancer treatment (Fritz et al., 2020). Our studies suggest that the histamine-HRH1 axis could serve as a potential biomarker of T cell dysfunction and immunotherapy response as well as promising therapeutic targets for enhancing immunotherapy response. The strong correlation between low levels of plasma histamine and better response to ICB treatment in cancer patients infers that patients who have high levels of histamine in plasma and thus respond poorly to immunotherapies may particularly benefit from antihistamine treatment. Based on our data, we consider that the low-cost OTC H1-antihistamines can be used as an adjuvant therapy in combination with immunotherapy to more effectively treat cancer patients.

HRH1 is also expressed in non-immune cells, including endothelial cells. In addition, histamine may disintegrate the endothelial barrier and induce vascular hyperpermeability (Ashina et al., 2015; Kugelmann et al., 2018). Our chimeric mice and macrophage co-implantation experiments indicated that HRH1 loss on TAMs is the major contributor to the enhanced immunity in HRH1−/− mice. However, HRH1−/− mice reconstituted with WT bone marrow (WT in HRH1−/−) also exhibited a slight tumor inhibition, suggesting that HRH1 loss in non-immune cells may also partly contribute to the tumor inhibition in HRH1−/− mice. Indeed, we found that compared with EO771 tumors in WT mice, tumors in HRH1−/− mice had reduced CD31+ blood vessel density, although they showed no significant difference in vascular permeability (data not shown). However, similar CD31+ blood vessel density was detected in FEXO-treated and vehicle-treated EO771 tumors. It is possible that HRH1−/− in endothelial cells induced impaired angiogenesis, but FEXO treatment only temporally blocks binding of histamine to HRH1 without significant effects on endothelial cells compared with those in HRH1−/− mice. Some phenotypes observed in HRH1−/− mice reconstituted with WT bone marrow may also be associated with reduced angiogenesis in HRH1−/− mice.

A major downstream effector of the histamine-HRH1 axis is VISTA, which has been implicated in ICB resistance in patients (Blando et al., 2019; Gao et al., 2017; Liu et al., 2015). Knockout of HRH1 gene or antihistamine treatment reduced membrane VISTA on TAMs and boosted T cell antitumor immunity, similar to anti-VISTA antibody. Recently, VISTA was identified as an acidic pH-selective ligand for the co-inhibitory receptor P-selectin glycoprotein ligand-1 (PSGL-1) on T cells, thus suppressing T cell function (Johnston et al., 2019). It was suggested that acidic pH, which is frequently found in TME, is required for VISTA to engage with PSGL-1 and suppress T cell immunity. Interestingly, we found that tumors growing in WT mice were more acidic compared with those in HRH1−/− mice (data not shown), which may favor binding of VISTA with PSGL-1 on T cells. Remarkably, when combined with ICB, antihistamines elicited a strong antitumor response superior to that of anti-VISTA antibody combined with ICB, suggesting that antihistamines also regulate other downstream effectors of immune stimulation/suppression, in addition to VISTA.

A most interesting finding from our studies is the potential impact of allergic reaction and histamine on antitumor immunity and immunotherapy response. Currently, studies regarding the relationship between allergy and cancer are controversial as regards epidemiological findings (Rittmeyer and Lorentz, 2012; Turner et al., 2006). Some studies suggested that allergies may reduce the risk of cancer either by increased immune surveillance after the immune hyperresponsiveness that may exert a protective effect against the development of cancer or by the physical effects of allergy symptoms that may inhibit cancer via removing potential carcinogens. In contrast, others suggested that the T helper cell type 2 response and inflammation induced by allergy may facilitate development of cancer. The relationship between allergy and cancer was unclear, since the potential impact of allergy on cancer had not been experimentally investigated so far. Here, our experimental data from both mammary tumor and colon cancer models in mice clearly demonstrated that allergy fueled tumor growth and triggered resistance to immunotherapy through histamine-HRH1-mediated suppression of antitumor immunity, highlighting the previously unrecognized tumor prone activity of allergy. Finally, our clinical data from ICB treated cancer patients indicate that pre-existing allergy with high plasma histamine impairs cancer patients' antitumor immune response and leads to their poor responses to immunotherapy.

Our clinical studies have a limitation that the numbers of patients with pre-existing allergies who received antihistamine treatment before ICB therapy were not recorded. Nevertheless, our finding that plasma histamine levels of, and uptake of antihistamines by, cancer patients are associated with their response to immunotherapy strongly supports using antihistamines to treat cancer patients who have allergy with high levels of plasma histamine. OTC H1-antihistamines can restore T cell function suppressed by cancer cell-secreted and/or allergy-released histamine and improve the efficacy of immunotherapies such as ICB therapies.

### Example 3. Impact of Medications, Including H1-Antihistamines, on Breast Cancer Recurrence

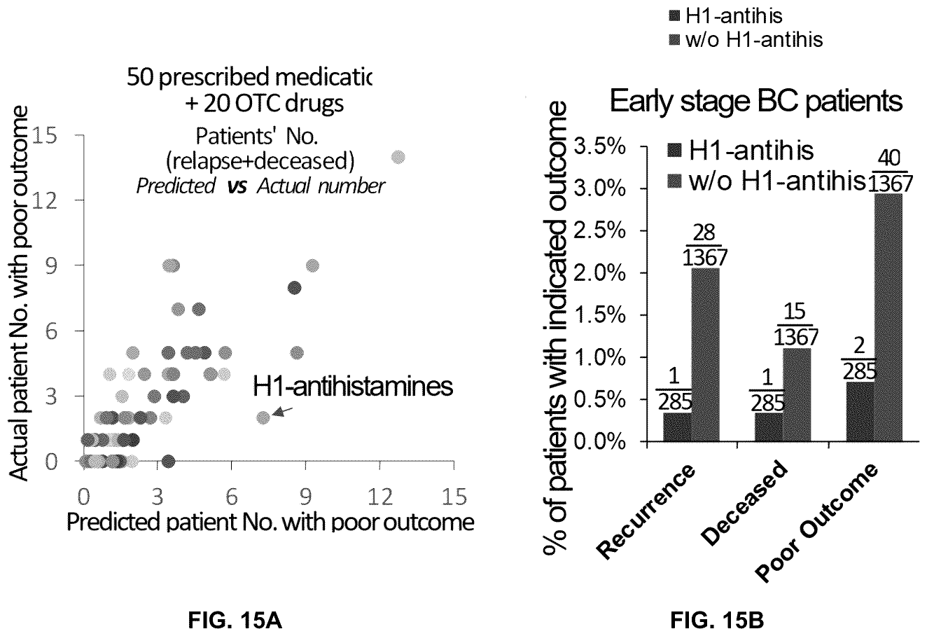

To explore whether some medications have impact on early-stage breast cancer recurrence, we performed a retrospective analysis of the most commonly prescribed medications took by patients with stage 0 or stage 1 breast cancer. Stage 0 and 1 breast cancers are highly treatable early-stage breast cancers and generally have very good prognosis in the first five years. Typically, surgery, radiation, or a combination of the two are the standard treatments. Hormone therapy, depending on the type of cancer cells and additional risk factors, is also an option. To rule out potential impact of the medications on therapies, patients who received treatment other than surgeries were excluded. 1652 patients diagnosed with stage 0 or 1 breast cancer at MDACC between 2016-2021 were enrolled in the study. 29 patients have been diagnosed with cancer recurrence (1.76%) and 3 out of 29 patients were deceased by 2021. Another 13 patients were also deceased, but whether cancer is the cause of their deaths is not clear. Altogether, total 42 patients (2.54%) have poor clinical outcome, including cancer recurrence or death. We first analyzed the 50 most prescribed drugs in USA which include many common drugs such as Aspirin, Simvastatin, and Metformin. About half of the drugs (24) were took by >100 patients based on their electric health record. To cover more medications, we further expanded the drug list by adding the top 20 over-the-counter medicines (OTC) sold on AMAZON website. To explore potential impact of medications on cancer recurrence and patient mortality, we compared the predicated patient numbers with poor clinical outcomes (X-axis) based on the estimated 2.54% (the ratio of total patients with poor outcome to total number of patients) versus the actual patient number with poor outcomes (Y-axis) of each medication (FIG. 15A). Most medications show limited impacts on patients' outcome, including Aspirin (8.5 vs. 8, estimated vs. actual patient number with poor outcome) and Metformin (3.5 vs 4). However, the 2nd generation antihistamines or H1-antihistamines is associated with significantly reduced number of patients' having poor clinical outcome (7.4 vs 2, Fisher test p=0.036), with only 1 patient diagnosed with cancer recurrence (0.35%) and 1 patient death among 285 patients taking the H1-antihistamines (FIG. 15B). These results suggest that H1-antihistamines may intercept early-stage breast cancer development. Importantly, patients who received therapies other than surgery were excluded from the study, indicating that the reduced recurrence and mortality observed in patients taking antihistamines is not due to enhanced therapeutic response. Instead, antihistamine may intercept or delay early-stage breast cancer progression by enhancing immune surveillance. Altogether, our studies strongly suggest that antihistamines may enhance anti-tumor immune response and improve efficacy of immunoprevention and immunotherapy.

### REFERENCES

1. Alquicira-Hernandez, J., et al. (2019). scPred: accurate supervised method for cell-type classification from single-cell RNA-seq data. Genome Biol. 20, 264.
2. Ashina, K., et al. (2015). Histamine induces vascular hyperpermeabil-ity by increasing blood flow and endothelial barrier disruption in vivo. PLoS ONE 10, e0132367.
3. Blando, J., et al. (2019). Comparison of immune infiltrates in melanoma and pancreatic cancer highlights VISTA as a potential target in pancreatic cancer. Proc. Natl. Acad. Sci. USA 116, 1692-1697.
4. Chen, L., et al. (2014). Metastasis is regulated via microRNA-200/ZEB1 axis control of tumour cell PD-L1 expression and intra-tumoral immunosuppression. Nat. Commun. 5, 5241.
5. De Henau, O., et al. (2016). Overcoming resistance to checkpoint blockade therapy by targeting PI3Kgamma in myeloid cells. Nature 539, 443-447.
6. DeNardo, D. G., and Ruffell, B. (2019). Macrophages as regulators of tumour immunity and immunotherapy. Nat. Rev. Immunol. 19, 369-382.
7. Elkrief, A., et al. (2019). The negative impact of antibiotics on outcomes in cancer patients treated with immunotherapy: a new independent prognostic factor?Ann. Oncol. 30, 1572-1579.
8. Faustino-Rocha, A. I., et al. (2017). Antihistamines as promising drugs in cancer therapy. Life Sci. 172, 27-41.
9. Fritz, I., et al. (2020). Desloratadine and loratadine use associated with improved melanoma survival. Allergy. https://doi.org/10.1111/all.14273.
10. Gao, J., et al., Efstathiou, E., et al. (2017). VISTA is an inhibitory immune checkpoint that is increased after ipilimumab therapy in patients with prostate cancer. Nat. Med. 23, 551-555.
11. Gerrick, K. Y., et al. (2018). Transcriptional profiling identifies novel regulators of macrophage polarization. PLoS One 13, e0208602.
12. Grauers Wiktorin, H., et al. (2019). Histamine targets myeloid-derived suppressor cells and improves the anti-tumor efficacy of PD-1/PD-L1 checkpoint blockade. Cancer Immunol. Immunother. 68, 163-174.
13. Guerriero, J. L. (2018). Macrophages: the road less traveled, changing anti¬cancer therapy. Trends Mol. Med. 24, 472-489.
14. Guerriero, J. L. (2019). Macrophages: their untold story in t cell activation and function. Int. Rev. Cell Mol Biol 342, 73-93.
15. Haak-Frendscho, M., et al. (2000). Histidine decarboxylase expression in human melanoma. J. Invest Dermatol. 115, 345-352.
16. Hao, Y., et al. (2021). Integrated analysis of multimodal single-cell data. Cell 184, 3573-3587.e3529.
17. Hugo, W., et al. (2016). Genomic and transcriptomic features of response to Anti-PD-1 therapy in metastatic melanoma. Cell 165, 35-44.
18. Jablonski, K. A., et al. (2015). Novel markers to delineate murine M1 and M2 macrophages. PLoS ONE 10, e0145342.
19. Jerby-Arnon, L., et al. (2018). A cancer cell program promotes t cell exclusion and resistance to checkpoint blockade. Cell 175, 984-997 e924.
20. Jiang, P., et al. (2018). Signatures of T cell dysfunction and exclusion predict cancer immunotherapy response. Nat. Med. 24, 1550-1558.
21. Johnston, R. J., et al. (2019). VISTA is an acidic pH-selective ligand for PSGL-1. Nature 574, 565-570.
22. Khononov, I., et al. (2021). Host response to immune checkpoint inhibitors contributes to tumor aggressiveness. J. Immunother. Cancer 9, e001996.
23. Kugelmann, D., et al (2018). Histamine causes endothelial barrier disruption via Ca(2+)-mediated RhoA activation and tension at adherens junctions. Sci. Rep. 8, 13229.
24. Kumar, R. K., et al. (2008). The “classical” ovalbumin challenge model of asthma in mice. Curr. Drug Targets 9, 485-494.
25. Le Mercier, I., et al. (2014). VISTA regulates the development of protective antitumor immunity. Cancer Res. 74, 1933-1944.
26. Li, T., et al. (2017). TIMER: a web server for comprehensive analysis of tumor-infiltrating immune cells. Cancer Res. 77, e108-e110.
27. Lines, J. L., et al. (2014). VISTA is an immune checkpoint molecule for human T cells. Cancer Res. 74, 1924-1932.
28. Liu, J., et al. (2015). Immune-checkpoint proteins VISTA and PD-1 nonredundantly regulate murine T-cell responses. Proc. Natl. Acad. Sci. USA 112, 6682-6687.
29. Lu, C., et al. (2010). Endothelial histamine H1 re¬ceptor signaling reduces blood-brain barrier permeability and susceptibility to autoimmune encephalomyelitis. Proc. Natl. Acad. Sci. USA 107, 18967-18972.
30. Martin, R. K., et al. (2014). Mast cell his¬tamine promotes the immunoregulatory activity of myeloid-derived suppres¬sor cells. J. Leukoc. Biol. 96, 151-159.
31. Martinez, F. O., et al. (2006). Transcriptional profiling of the human monocyte-to-macrophage differentia¬tion and polarization: new molecules and patterns of gene expression. J. Immunol. 177, 7303-7311.
32. Massari, N. A., et al. (2018). Histamine receptors and cancer pharmacology: an update. Br. J. Pharmacol. 177, 516-538.
33. Micaroni, M. (2010). The role of calcium in intracellular trafficking. Curr. Mol. Med. 10, 763-773.
34. Micaroni, M. (2012). Calcium around the Golgi apparatus: implications for intracellular membrane trafficking. Adv. Exp. Med. Biol. 740, 439-460.
35. Misharin, A. V., et al. (2013). Flow cytometric analysis of macrophages and dendritic cell subsets in the mouse lung. Am. J. Respir. Cell Mol. Biol. 49, 503-510.
36. Moriarty, C. M., et al. (1988). Blood histamine and solid malignant tumors. J. Cancer Res. Clin. Oncol. 114, 588-592.
37. Mosely, S. I., et al. (2017). Rational selection of syngeneic preclinical tumor models for immunotherapeutic drug discovery. Cancer Immunol. Res. 5, 29-41.
38. Newman, A. M., et al. (2015). Robust enumeration of cell subsets from tissue expression profiles. Nat. Methods 12, 453-457.
39. Nials, A. T., and Uddin, S. (2008). Mouse models of allergic asthma: acute and chronic allergen challenge. Dis. Model. Mech. 1, 213-220.
40. Nowak, E. C., et al. (2017). Immunoregulatory functions of VISTA. Immunol. Rev. 276, 66-79.
41. Ocana-Guzman, R., et al. (2016). TIM-3 regulates distinct functions in macrophages. Front. Immunol. 7, 229.
42. Orecchioni, M., et al (2019). Macrophage polarization: different gene signatures in M1(LPS+) vs. classically and M2(LPS−) vs. alternatively activated macrophages. Front. Immunol. 10, 1084.
43. Ostuni, R., et al. (2015). Macrophages and cancer: from mechanisms to therapeutic implications. Trends Immunol. 36, 229-239.
44. Parsons, M. E., and Ganellin, C. R. (2006). Histamine and its receptors. Br. J. Pharmacol. 147, S127-S135.
45. Ribas, A., and Wolchok, J. D. (2018). Cancer immunotherapy using checkpoint blockade. Science 359, 1350-1355.
46. Rittmeyer, D., and Lorentz, A. (2012). Relationship between allergy and cancer: an overview. Int. Arch. Allergy Immunol. 159, 216-225.
47. Sharma, P., et al. (2017). Primary, adaptive, and acquired resistance to cancer immunotherapy. Cell 168, 707-723.
48. Sieja, K., et al. (2005). Concentration of histamine in serum and tissues of the primary ductal breast cancers in women. Breast 14, 236-241.
49. Simons, F. E. (2004). Advances in H1-antihistamines. N. Engl. J. Med. 351, 2203-2217.
50. Thurmond, R. L., et al. (2008). The role of histamine H1 and H4 receptors in allergic inflammation: the search for new antihista¬mines. Nat. Rev. Drug Discov. 7, 41-53.
51. Turner, M. C., et al. (2006). An overview of the association between allergy and cancer. Int. J. Cancer 118, 3124-3132.
52. Uhlen, M., et al. (2017). A pathology atlas of the human cancer transcriptome. Science 357, eaan2507.
53. Verma, V., et al. (2019). PD-1 blockade in sub-primed CD8 cells induces dysfunctional PD-1(+)CD38(hi) cells and anti-PD-1 resistance. Nat. Immunol. 20, 1231-1243.
54. von Mach-Szczypinski, J., et al. (2009). Metabolism of histamine in tissues of primary ductal breast cancer. Metabolism 58, 867-870.
55. Yang, X. D., et al. (2011). Histamine deficiency pro¬motes inflammation-associated carcinogenesis through reduced myeloid maturation and accumulation of CD11b+Ly6G+ immature myeloid cells. Nat. Med. 17, 87-95.
56. Yao, J., et al. (2012). Identification of common prognostic gene expression signatures with biological meanings from microarray gene expression datasets. PLoS One 7, e45894.
57. Zelenay, S., et al. (2015). Cyclooxygenase-dependent tumor growth through evasion of immu¬nity. Cell 162, 1257-1270.
58. Zhang, L., et al. (2020). Blocking immunosuppressive neu-trophils deters pY696-EZH2-driven brain metastases. Sci. Transl Med. 12, eaaz5387.
59. Zhang, L., et al. (2015). Microenvironment-induced PTEN loss by exosomal microRNA primes brain metastasis outgrowth. Nature 527, 100-104.
60. Zhang, X., et al. (2019). CellMarker: a manually curated resource of cell markers in human and mouse. Nucl. Acids Res. 47, D721-D728.
61. Zheng, G. X., et al. (2017). Massively parallel digital transcriptional profiling of single cells. Nat. Commun. 8, 14049.

The above description of example embodiments of the present disclosure has been presented for the purposes of illustration and description. It is not intended to be exhaustive or to limit the disclosure to the precise form described, and many modifications and variations are possible in light of the teaching above.

A recitation of “a”, “an” or “the” is intended to mean “one or more” unless specifically indicated to the contrary. The use of “or” is intended to mean an “inclusive or,” and not an “exclusive or” unless specifically indicated to the contrary. Reference to a “first” component does not necessarily require that a second component be provided. Moreover, reference to a “first” or a “second” component does not limit the referenced component to a particular location unless expressly stated. The term “based on” is intended to mean “based at least in part on.”

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

All patents, patent applications, publications, and descriptions mentioned herein are incorporated by reference in their entirety for all purposes. None is admitted to be prior art. Where a conflict exists between the instant application and a reference provided herein, the instant application shall dominate.

When a group of substituents is disclosed herein, it is understood that all individual members of those groups and all subgroups and classes that can be formed using the substituents are disclosed separately. When a Markush group or other grouping is used herein, all individual members of the group and all combinations and subcombinations possible of the group are intended to be individually included in the disclosure. As used herein, “and/or” means that one, all, or any combination of items in a list separated by “and/or” are included in the list; for example “1, 2 and/or 3” is equivalent to “‘1’ or ‘2’ or ‘3’ or ‘1 and 2’ or ‘1 and 3’ or ‘2 and 3’ or ‘1, 2 and 3’”. Whenever a range is given in the specification, for example, a temperature range, a time range, or a composition range, all intermediate ranges and subranges, as well as all individual values included in the ranges given are intended to be included in the disclosure.

1-19. (canceled)

20. A method of treating cancer in a subject, comprising
determining that the subject has at least one of an elevated level of histamine or HRH1 signaling activation, and
administering to the subject an antihistamine in conjunction with an immunotherapy comprising the administration of a checkpoint inhibitor.

21. The method of claim 20, wherein the checkpoint inhibitor is an inhibitor of PD-1, PD-L1, or CTLA-4.

22. The method of claim 20, wherein the subject is determined to have an elevated level of histamine by detecting histamine in a plasma sample obtained from the subject, and/or by detecting histamine, HDC mRNA, HDC protein, and/or mast cells in a tumor sample obtained from the subject.

23. The method of claim 20, wherein the subject is determined to have an elevated level of HRH1 signaling activation by detecting the level of HRH1 mRNA, HRH1 protein, HRH1+ macrophages, VISTA+ macrophages, PI3K-gamma+ macrophages, and/or TIM-3+ macrophages in a tumor sample obtained from the subject.

24. The method of claim 20, wherein the level of histamine or HRH1 signaling activation is detected by immunohistochemistry (IHC) staining, flow cytometry staining, and/or ELISA.

25. The method of claim 20, wherein the subject is determined to have an elevated level of histamine or HRH1 signaling activation by virtue of the presence of allergies and/or by detection of plasma IgE levels in the subject.

26. The method of claim 20, wherein the antihistamine is an H1-antihistamine.

27. (canceled)

28. The method of claim 20, wherein the derivative is an intravenous injection form of the H1-antihistamine.

29. (canceled)

30. The method of claim 20, wherein the antihistamine is an inhibitor of HDC-mediated histamine production or of a downstream effector of HRH1 activation.

31. (canceled)

32. The method of claim 20, wherein the cancer is associated with high levels of histamine production, HRH1 signaling activation, and/or plasma histamine.

33. The method of claim 20, wherein the cancer is colorectal cancer, breast cancer, lung cancer, or malignant melanoma.

34. A method of generating a report containing information on the likelihood that a subject with cancer will respond to an immunotherapy comprising a checkpoint inhibitor, comprising:
detecting a level of histamine and/or HRH1 signaling activation in a biological sample obtained from the subject; and,
generating the report,
wherein the report is useful for determining the likelihood that the subject will respond to the therapy.

35-36. (canceled)

37. The method of claim 34, wherein the biological sample is a tumor sample, and wherein the level of histamine and/or HRH1 signaling activation is determined by detecting histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages in the sample.

38-42. (canceled)

43. A method of treating a subject with cancer, the method comprising administering to the subject a therapeutically effective amount of a checkpoint inhibitor and of an antihistamine, wherein the subject has been identified as unlikely to respond to an immunotherapy comprising the checkpoint inhibitor based on a detection of levels of histamine and/or HRH1 signaling activation in a biological sample obtained from the subject, wherein the identification of the subject as unlikely to respond to the immunotherapy is based on a difference in the level of histamine and/or HRH1 signaling activation in the biological sample obtained from the subject as compared to the level in a biological sample obtained from an individual known to be responsive to the immunotherapy.

44. The method of claim 43, wherein the identification of the subject as unlikely to respond to the immunotherapy comprises the calculation of a response score based on the detected levels of histamine and/or HRH1 signaling activation in the biological sample, wherein the response score corresponds to the likelihood that the subject will respond to the immunotherapy.

45. The method of claim 43, wherein the checkpoint inhibitor is an inhibitor of PD-1, PD-L1, or CTLA-4.

46-47. (canceled)

48. The method of claim 43, wherein the biological sample is a tumor sample, and wherein the level of histamine and/or HRH1 signaling activation is determined by detecting histamine, HDC mRNA, HDC protein, mast cells, HRH1 mRNA, HRH1 protein, HRH1+ macrophages, VISTA+ macrophages, PI3K-gamma+ macrophages, and/or TIM-3+ macrophages in the sample.
